(19) United States
(12) Patent Application Publication
(10) Pub. No.: US 2015/0191430 A1
(43) Pub. Date: Jul. 9, 2015

(54) CONJUGATES OF HUPERZINE AND ANALOGS THEREOF

(71) Applicant: INSERO HEALTH INC., Miami, FL (US)

(72) Inventors: David Kolb, Miami, FL (US); Seth Herzon, Miami, FL (US)

(21) Appl. No.: 14/399,063

(22) PCT Filed: May 3, 2013
(86) PCT No.: PCT/US13/39587

§ 371 Date: Nov. 5, 2014

Related U.S. Application Data
(60) Provisional application No. 61/649,052, filed on May 18, 2012.
(60) Provisional application No. 61/665,643, filed on Jun. 28, 2012.
(60) Provisional application No. 61/723,257, filed on Nov. 6, 2012.

(51) Int. Cl. C07D 215/227; A61K 45/06; C07D 401/12; C07D 413/12; A61K 31/4748; C07D 417/12

(52) U.S. Cl. CPC C07D 215/227; A61K 31/4748; C07D 417/12; C07D 401/12; C07D 413/12; A61K 45/06

(57) ABSTRACT

Compounds and compositions for treating neurodegenerative diseases are described. The compounds include a therapeutic agent covalently linked with huperzine or an analog thereof through a linker. Methods of preparing the compounds are described. Methods of treating a neurodegenerative disease by administering compounds and compositions including a therapeutic agent covalently linked with huperzine or an analog thereof are described. Methods for delivering a therapeutic agent by administering the therapeutic agent covalently linked to huperzine or an analog thereof are described.

### CROSS-REFERENCE TO RELATED APPLICATIONS

This application claims priority to U.S. Provisional. Application No. 61/649,052 entitled “Conjugates of Huperzine and Analogs Thereof” filed May 18, 2012, U.S. Provisional Application No. 61/665,643 entitled “Conjugates of Huperzine and Analogs Thereof” filed Jun. 28, 2012, and U.S. Provisional Application No. 61/723,257 entitled “Conjugates of Huperzine and Analogs Thereof” filed Nov. 6, 2012, which are herein incorporated by reference in their entirety.

### SUMMARY OF THE INVENTION

Embodiments of the present invention are conjugates of huperzine or huperzine analogs having a general formula (I) linked with at least one of an N-methyl-D-aspartate (“NMDA”) receptor antagonist, a mitochondrial protectant, an anti-inflammatory agent, an anticonvulsant, or an anxiolytic agent. The conjugates provide huperzine or huperzine analog and a therapeutic agent in a specific delivery to brain tissue for the alleviation or amelioration of pathological disease states in the brain. Thus, the present invention provides methods and compositions of matter for facilitating the transit of such conjugates of psychotropic, neurotropic or neurological drugs, agents and compounds across the blood-brain barrier and into targeted regions of the brain, for the treatment of animal, preferably human, diseases and pathological conditions.

### DETAILED DESCRIPTION

Before the present compositions and methods are described, it is to be understood that this invention is not limited to the particular processes, compositions, or methodologies described, as these may vary. It is to be also understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

Optical isomers—diastereomers—geometric isomers—tautomers. Compounds described herein may contain an asymmetric center and may thus exist as enantiomers. Where the compounds according to the invention possess two or more asymmetric centers, they may additionally exist as diastereomers. The present invention includes all possible stereoisomers as substantially pure resolved enantiomers, racemic mixtures thereof, as well as mixtures of diastereomers. The formulas are show without a definitive stereochemistry at certain positions. The present invention includes all stereoisomers of such formulas and pharmaceutically acceptable salts and solvates thereof. Diastereoisomeric pairs of enantiomers may be separated by, for example, fractional crystallization from a suitable solvent, and the pair of enantiomers thus obtained may be separated into individual stereoisomers by conventional means, for example, by use of an optically active acid or base or a resolving agent or on a chiral HPLC column. Further, any enantiomer or diastereomer of a compound of the general formula may be obtained by stereospecific using optically pure starting materials or reagents of known configuration.

It must be noted that as used herein and in the appended claims, the singular limns “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 50% means in the range of 40%-60%.

“Administering” when used in conjunction with a therapeutic means to administer a therapeutic agent into or onto a target tissue or to administer a therapeutic to a patient whereby the therapeutic agent positively impacts the tissue to which it is targeted.

The terms “individual”, “host”, “subject”, “patient”, and “animal” as used interchangeably herein include but are not limited to, humans and non-human vertebrates such as wild, domestic and farm animals.

The term “improves” as used herein, is used to convey that the present invention changes either the appearance, form, characteristics, physiological, and/or the physical attributes of the tissue and/or organ to which it is being provided, applied or administered.

The term “inhibiting” includes the administration of a compound of the present invention to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, the term “therapeutic agent” means an agent utilized to treat, combat, ameliorate, prevent or improve an unwanted condition or disease of a patient. In part, embodiments of the present invention are directed to the treatment of neurodegenerative disease or decrease the symptoms thereof. Therapeutic agents of the present inventions include drugs, pro-drugs, and precursors that can be activated when the therapeutic agent is delivered to the target tissue.

A “therapeutically effective amount” or “effective amount” of a composition is a predetermined amount calculated to achieve the desired effect. The activity contemplated by the present methods includes both medical therapeutic and/or prophylactic treatment, as appropriate. The specific dose of a compound administered according to this invention to obtain therapeutic and/or prophylactic effects, will, of course, be determined by the particular circumstances surrounding the ease, including, for example, the compound administered, the mute of administration, and the condition being treated. The compounds are effective over a wide dosage range and, for example, dosages per day will normally fall within the range of from 0.001 to 10 mg/kg, more usually in the range of from 0.01 mg/kg to 1 mg/kg. However, it will be understood that the effective amount administered will be determined by the physician in the light of the relevant circumstances including the conditions to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Many geometric isomers of olefins, C═N double bonds, N═N double bonds, amides, and the like can also be present in the compounds described herein, and all such stable isomers are contemplated. Cis and trans geometric isomers of the compounds are also contemplated and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

The terms “treat”, “treated”, or “treating” as used herein refer to both therapeutic treatment and preventative measures, wherein the object is to prevent or slow down an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization of the state of the condition, disorder or disease; delay in onset or slowing of the progression of the condition, disorder or disease; amelioration of the condition, disorder or disease state; and remission (whether partial or total), Whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The terms “carrier”, “excipient”, “diluent”, and “adjuvant” may be used interchangeably and refer to a composition with which the therapeutic agent is administered. Such carriers may be sterile liquids such as, for example, water and oils, including those of petroleum, animal, vegetable or synthetic origin. Saline solution, aqueous dextrose and glycerol solution may also be employed as liquid carriers. Suitable pharmaceutical excipients include, but are not limited to, glucose, starch, lactose, sucrose, gelatin, malt, rice, flour, chalk, sodium chloride, dried skim milk, glycerol, propylene, glycol, water, and ethanol. The composition, if desired, may contain minor amounts of wetting or emulsifying agents, or pH buffering agents. These compositions may take a form of solutions, suspensions, emulsions, powders, sustained-release formulations, and the like.

The term “alkyl,” as used herein, refers to a branched or unbranched saturated hydrocarbon group of 1 to 24 carbon atoms, such as, without limitation, methyl, ethyl, n-propyl, isopropyl, n-butyl, isobutyl, tert-butyl, pentyl, hexyl, heptyl, octyl, decyl and the like. Preferred alkyl groups herein contain 1 to 6 carbon atoms. Alkyls may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkynyl, C₁-C₆haloalkyl, C₁-C₆haloalkoxy, amino(C₁-C₆) alkyl, mono- or di- or tri(C₁-C₆alkylamino(C₀-C₆alkyl), C₀-C₆thioalkyl, C₀-C₆alkylulfonyl, aryl, heterocyclyl and heteroaryl.

The term “alkenyl,” as used herein, refers to a branched or unbranched hydrocarbon group of 1 to 24 carbon atoms containing at least one unsaturated bond, such as, without limitation, vinyl, propenyl, butenyl, pentenyl, hexenyl, heptenyl, octenyl, decenyl, and the like. Preferred alkenyl groups herein contain 1 to 6 carbon atoms. Alkenyl groups may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkenyl, C₂-C₆alkynyl, C₁-C₆haloalkyl, C₁-C₆haloalkoxy, amino(C₁-C₆) alkyl, mono- or di- or tri(C₁-C₆alkylamino(C₀-C₆alkyl), C₀-C₆thioalkyl, C₀-C₆alkylulfonyl, aryl, heterocyclyl and heteroaryl.

The term “alkynyl,” as used herein, refers to a branched or =branched hydrocarbon group of 1 to 24 carbon atoms containing at least one triple bond, such as, without limitation, acetylenyl, propynyl, butynyl, pentynyl, hexynyl, heptynyl, octynyl, decynyl, and the like. Preferred alkynyl groups herein contain 1 to 6 carbon atoms. Alkynyl groups may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkenyl, C₂-C₆alkynyl, C₁-C₆haloalkyl, C₁-C₆haloalkoxy, amino(C₁-C₆) alkyl, mono- or di- or tri(C₁-C₆alkylamino(C₀-C₆alkyl), C₀-C₆thioalkyl, C₀-C₆alkylulfonyl, aryl, heterocyclyl and heteroaryl.

The term “cycloalkyl” refers to ring-containing alkyl radicals. Examples include cyclohexyl, cyclopentyl, cyclopropyl, cyclopropylmethyl and norbornyl. Cycloalkyl groups may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkenyl, C₂-C₆alkynyl, C₁-C₆haloalkyl, C₁-C₆haloalkoxy, amino(C₁-C₆) alkyl, mono- or di- or tri(C₁-C₆alkylamino(C₀-C₆alkyl), C₀-C₆thioalkyl, C₀-C₆alkylulfonyl, aryl, heterocyclyl and heteroaryl.

The term “aryl” or “Ar” employed alone or in combination with other terms, means, unless otherwise stated, a carbocyclic aromatic group containing one or more rings (typically one, two or three rings). Multiple rings may be attached together in a pendent manner, such as a biphenyl, or may be fused, such as naphthalene. Examples include, but are not limited to, phenyl, anthracyl and naphthyl. Preferred are phenyl (Ph) and naphthyl, most preferred is phenyl. Aryl groups may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkenyl, C₂-C₆alkynyl, C₁-C₆haloalkyl, C₁-C₆haloalkoxy, amino(C₁-C₆) alkyl, mono- or di- or tri(C₁-C₆alkylamino(C₀-C₆alkyl), C₀-C₆thioalkyl, C₀-C₆alkylulfonyl, aryl, heterocyclyl and heteroaryl.

The term “heterocycle”. “heterocyclyl” or “heterocyclic” by itself or as part of another substituent means, unless otherwise stated, an unsubstituted or substituted, stable, mono- or multicyclic heterocyclic ring system consisting of carbon atoms and at least one heteroatom selected from the group consisting of N, O, and S, and wherein the nitrogen and sulfur heteroatoms may be optionally oxidized, and the nitrogen atom may be optionally quaternized. The heterocycle may be attached to the compound of which it is a component, unless otherwise stated, at any heteroatom or carbon atom in the heterocycle that affords a stable structure. Heterocyclic groups may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkenyl, C₂-C₆alkynyl, C₁-C₆haloalkyl, C₁-C₆haloalkoxy, amino(C₁-C₆) alkyl, mono- or di- or tri(C₁-C₆alkylamino(C₁-C₆alkyl), C₀-C₆thioalkyl, C₀-C₆alkylulfonyl, aryl, heterocyclyl and heteroaryl.

Examples of non-aromatic heterocycles include, but are not limited to, monocyclic groups such as: aziridinyl, oxiranyl, thiiranyl, azetidinyl, oxetanyl, thietanyl, pyrrolidinyl, pyrrolinyl, imidazolinyl, pyrazolidinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, 1,4-dihydropyridinyl, piperidinyl, morpholinyl, thiomorpholinyl, pyranyl, 2,3-dihydropyranyt, tetrahydropyranyl, 1,4-dioxanyl, 1,3-dioxanyl, homopiperazinyl, homopiperidinyl, 1,3-dioxepinyl, 4,7-dihydro-1,3-dioxepinyl and hexamethyleneoxide.

The term “heteroaryl” or “heteroaromatic” refers to a heterocycle having aromatic character. A monocyclic heteroaryl group is preferably a 5-, 6-, or 7-membered ring, examples of which are pyrrolyl, furyl, thienyl, pyridyl, pyrimidinyl and pyrazinyl. A polycyclic heteroaryl may comprise multiple aromatic rings or may include one or more partially saturated rings. Heteroaryl groups may be optionally substituted in suitable positions with, for example, C₁-C₆ alkyl, C₁-C₆ alkoxy, halogen, hydroxy, cyano, nitro, amino, mono- or di-(C₂-C₆)alkylamino, C₂-C₆alkenyl, C₂-C₆alkynyl, C₁-C₆haloalkyl, C₁-C₆haloalkoxy, amino(C₁-C₆) alkyl, mono- or di- or tri(C₁-C₆alkylamino(C₀-C₆alkyl), C₀-C₆thioalkyl, C₀-C₆alkylulfonyl, aryl, heterocyclyl and heteroaryl.

Examples of monocyclic heteroaryl groups include, for example, six-membered monocyclic aromatic rings such as, for example, pyridyl, pyrazinyl, pyrimidinyl and pyridazinyl; and five-membered monocyclic aromatic rings such as, for example, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heteroaryl groups containing a partially saturated ring include tetrahydroquinolyl and 2,3-dihydrobenzofuryl. Other examples of polycyclic heteroaryls include indolyl, indolinyl, quinolyl, tetrahydroquinolyl, isoquinolyl, 1,2,3,4-tetrahydroisoquinolyl, cinnolinyl, quinoxalinyl, quinazolinyl, phthalazinyl, 1 naphthyridinyl, 1,4-benzodioxanyl, chromene-2-one-yl (coumarinyl), dihydrocoumarin, chromene-4-one-yl, benzofuryl, 1,5-naphthyridinyl, 2,3-dihydrobenzofuryl, 1,2-benzisoxazolyl, benzothienyl, benzoxazolyl, benzothiazolyl, purinyl, benzimidazolyl, benzotriazolyl, thioxanthinyl, benzazepinyl, benzodiazepinyl, carbazolyl, carbolinyl, acridinyl, pyrrolizidinyl and quinolizidinyl.

The term “substituted” refers to a molecular group that replaces a hydrogen in a compound.

As used herein, the term “neurodegenerative disease” refers to conditions or symptoms resulting from progressive loss of structure or function of neurons, including neuronal death. Conditions or diseases that can be classified as neurodegenerative include, but are not limited to, Alzheimer's disease, epilepsy, Parkinson's disease. Huntington's disease, neuropathic pain, multiple sclerosis, ataxia, amyotrophic lateral sclerosis, AIDS-related dementia, neurotoxic poisoning, muscular dystrophy, myasthenia gravis, vascular dementia, glaucoma, orthostatic hypotension, mitochondrial diseases, and infantile spasms.

The term “co-administration,” when used herein the compounds may be combined in one pharmaceutically-acceptable carrier, or they may be placed in separate carriers and administered to the patient at different times. Those of skill in the art understand that the formulations and/or routes of administration of the various agents/therapies used may vary. The appropriate dosage for co-administration can be readily determined by one skilled in the art. The important consideration being that the compounds should be administered sufficiently dose in time that there is at least some temporal overlap in the biological effects generated by the compounds into the mammal being tested.

Huperzine is a naturally occurring sesquiterpene alkaloid compound that acts as an acetylcholinesterase inhibitor (AChEI) as well as an N-methyl d-aspartate (NMDA) receptor antagonist. Huperzine has an acetylcholinesterase inhibition mechanism of action similar to compounds such as donepezil, rivastigmine, and galantamine, and is being studied for use in treating Alzheimer's disease. As an NMDA receptor antagonist, huperzine also purportedly protects the brain against glutamate induced damage. Because of its dual action, huperzine may have uses in treatment of other neurodegenerative diseases.

Huperzine A has the following structure (1) wherein n is 1, R₁ is CH₃, R₂ is CH₃, R_{N1} is H, R_{N2} is H, R_{N3} is absent, R₄ is absent, and R₅ is H.

A first aspect includes compounds comprising conjugates of huperzine or analog of huperzine having the general formula:

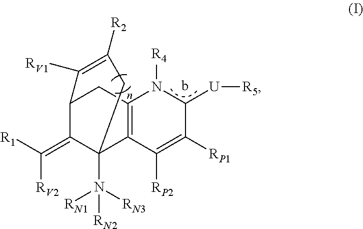

tautomer thereof, or pharmaceutically acceptable salt thereof wherein R₁ is selected from H, (C₁-C₂₄)alkyl, CF₃, CF₂CF₃, CF₂CF₂CF₃, SO₂CH₃, SO₂Ph, SO₂Ar, SO₃H, and SO₃Ar, and —CH₂-L-T; R₂ is selected from H, (C₁-C₂₄)alkyl, aryl, cycloalkyl, (C₂-C₂₄)alkenyl, heterocycle, heteroaryl and —CH₂-L-T; R_{P1}, R_{P2}, R_{V1}, R_{V2} are independently selected from hydrogen and fluorine; R_{N1} is selected from H, (C₁-C₂₄)alkyl, CF₃, CF₂CF₃, CCl₃, CBr₃, CHO, and -L-T; R_{N1} and R_{N2} is selected from H, (C₁-C₂₄)alkyl, CF₃, CF₂CF₃, CCl₃, CBr₃, and CHO; R_{N3} is selected from absent and (C₁-C₂₄)alkyl; U is O, S, NH, or N((C₁-C₂₄)alkyl); h is a keto-enol tautomer unsaturation; and n is an integer selected front 1, 2, 3, and 4; R₄ is absent, or selected from H, and -L-T; R₅ is absent, or selected from H, and -L-T; at least one of R₁, R₂, R_{N1}, R₄ and R₅ is -L-T; each -L- is independently a linker; and each -T is independently selected front a therapeutic agent, a therapeutic agent pro-drug, or a therapeutic agent precursor.

In some embodiments, the linker (L) may comprise at least one of a linker functional group selected from a bond, —O—, —S—, —NH—, —N(alkyl)-, —C(O)—, —O(C═O)—, —C(═O)O—, —C(═S)O—, —C(═S)—, and —P(O)₂—. In various embodiments, the linker is a bond. In other embodiments, the linker is —C(O)—. In other embodiments, the linker is —C(═S)—. In still other embodiments, the linker is —P(O)₂—. In yet other embodiments, the linker is an ether, sulfide, or an amine. In some embodiments, the linker is —O(C═O)—, or in other embodiments, —C(═O)O—.

In some embodiments, each therapeutic agent, a therapeutic went pro-drug, or a therapeutic agent precursor is independently —V—W—X—Y—Z, wherein V is bond, —O—, or —NH—; W is —(C₀-C₆)alkyl-, —(C₂-C₆)alkenyl-; or —(C₂-C₆)alkynyl-; X is a bond, —O—, —NH—, —CO—, —(C═O)NH—, —NH—(C═O)—, —SO₂—, —(C═NH)—NH—, —(C═O)—O—, or —O(C═O)—; Y is a —(C₀-C₆)alkyl-, —(C₂-C₆)alkenyl-; or —(C₂-C₆)alkynyl-; Z is a -quaternary amine, -cycloalkyl, -heterocycle, or heteroaryl; and each nitrogen, alkyl, alkenyl, alkynyl, cycloalkyl, heterocycle, or heteroaryl is optionally substituted.

In some embodiments of the compounds, only one of R_{N1}, R₄ and R₅ is -L-T; R₁ is one of H, CH₃, CF₃, CF₂CF₃, CF₂CF₂CF₃, SO₂CH₃, SO₂Ph, SO₂Ar, or SO₃H; and R₂ is one of H, (C₁-C₂₄)alkyl, aryl, cycloalkyl, (C₂-C₂₄)alkenyl, heterocycle, or heteroaryl. In some embodiments, n=1, and R₁═R₂═CH₃.

In some embodiments, R_{P1} is H or F; R_{P2} is H or F; R_{V1} is H or F; R_{V2} is H or F; wherein at least one of R_{P1}, R_{V1}, and R_{V2} is fluorine. In another embodiment, R_{P1} is H or F; R_{P2} is H or F; R_{V1} is H or F; R_{V2} is H or F; wherein at least one of R_{P1}, R_{P2}, R_{V1}, and R_{V2} is fluorine; R₁ is methyl; R₂ is methyl; R_{N1} is H; R_{N2} is H; and R_{N3} is absent.

In some embodiments, -T is —(C═O)—CH₂—CH(i-Bu)-CH₂—NH₂, (S)—(C═O)—CH₂—CH(i-Bu)-CH₂—NH₂, In other embodiments, -T is (S)—(C═O)—CH₂—CH(i-Bu)-CH₂—NH₂.

In still other embodiments, the compound is one where -T is a covalently bonded glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl d-aspartate receptor antagonist, mitochondrial protectant, an anti-inflammatory agent, alpha-7 agonist, potassium channel blacker, sodium channel blocker, anticonvulsant, anxiolytic agent, NADPH oxidase inhibitor; a precursor to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor, a gamma amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof.

In yet other embodiments, -T is a covalently bonded N-methyl d-aspartate receptor antagonist selected from R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantadine, dextrallorphan, dextromethorphan, dextrorphan, dizocilpine, eticyclidine, gacyclidine, ibogaine, memantine, methoxetamine, phencyclidine, rolicyclidine, tenocyclidine, methoxydine, tiletamine, neramexane, eliprodil, etoxadrol, dexoxadrol, remacemide, delucemine, 8a-phenyldecahydroquinoline, aptiganel, rhynchophylline, ketamine, 1-aminocyclopropanecarboxylic acid, 7-chlorokynurenate, 5,7-dichlorokynurenic acid, kynurenic acid, lacosamide; a pro-drug of the NMDA receptor antagonist, a precursor of the NMDA receptor antagonist; and any combination thereof.

In yet other embodiments, the compound is one wherein -T is any one of the structures XLXXIX-CXVIII, or pharmaceutically acceptable salt thereof:

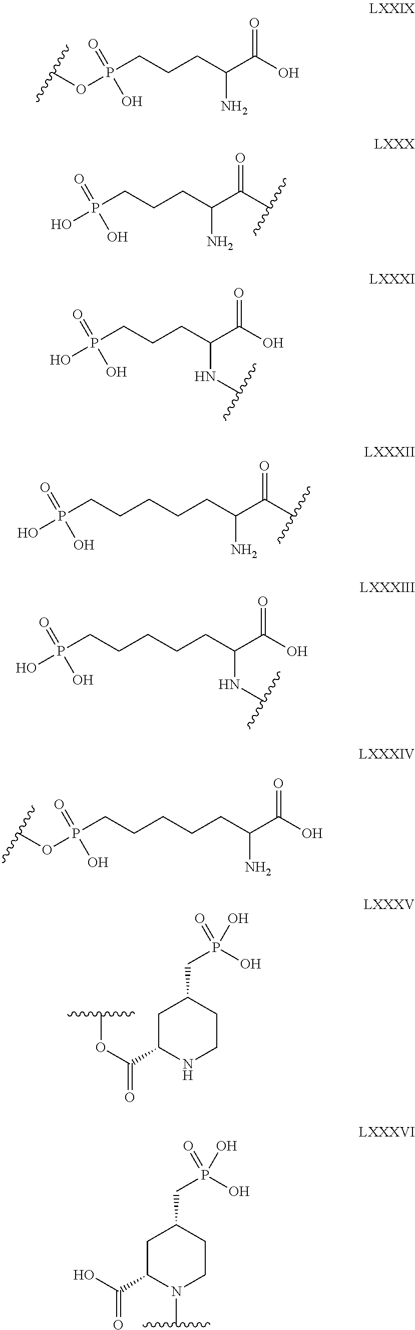

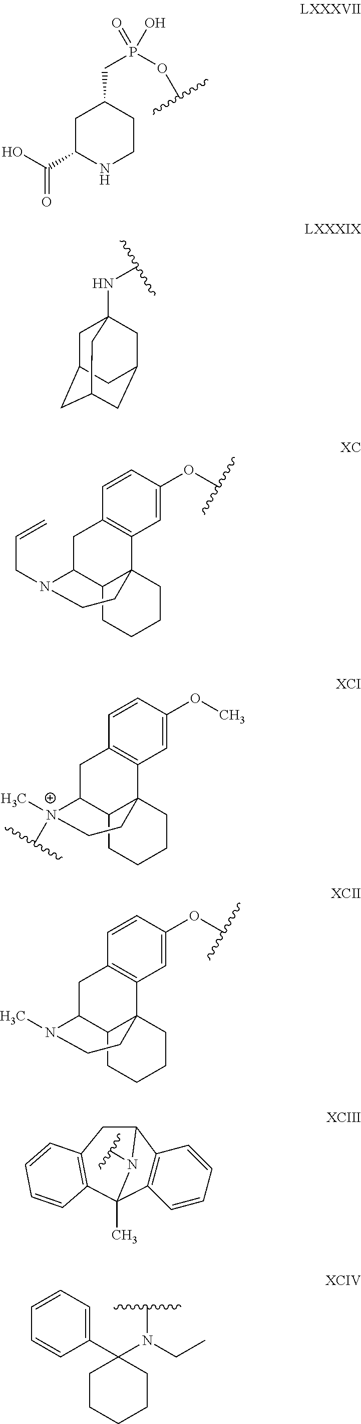

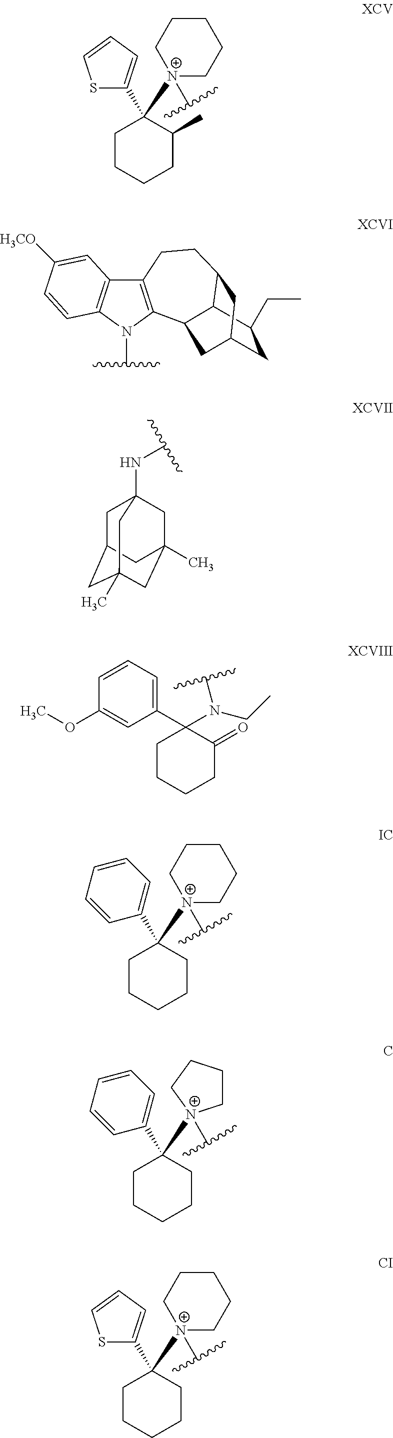

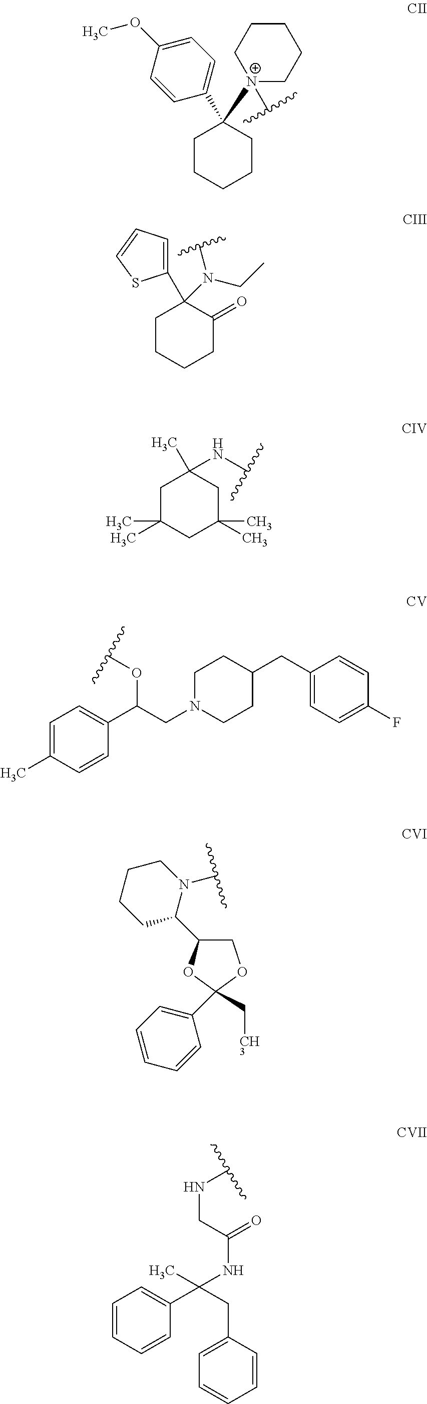

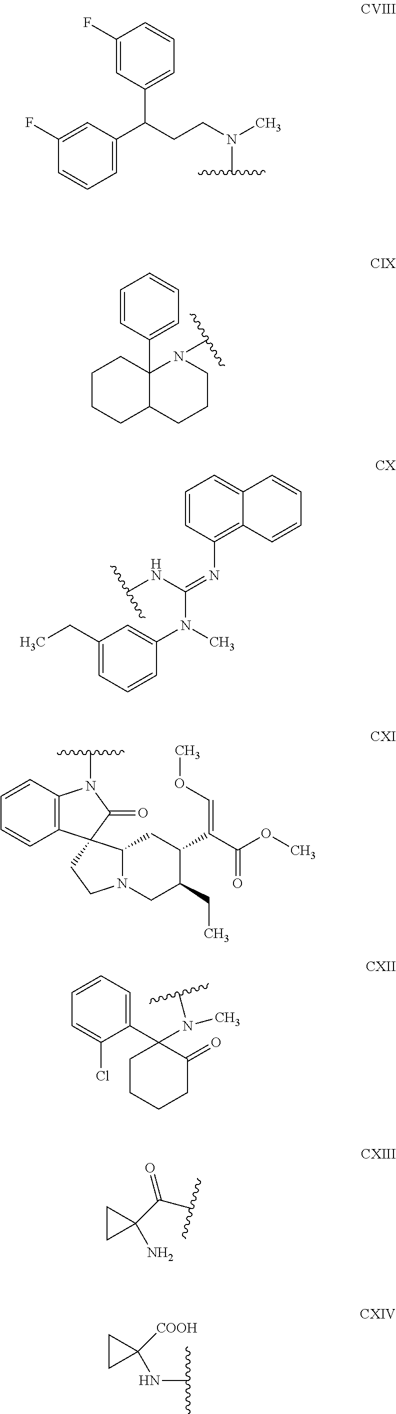

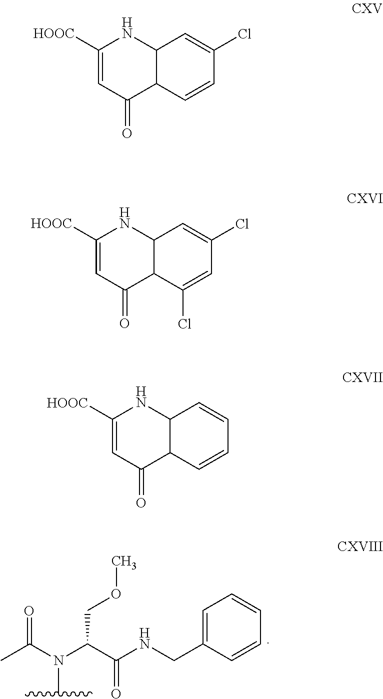 [SMILES: O=C(O)C1=CC(=O)C2C=CC(Cl)=CC2N1 ; O=C(O)C1=CC(=O)C2C(Cl)=CC(Cl)=CC2N1 ; O=C(O)C1=CC(=O)C2C=CC=CC2N1 ; *N(C(C)=O)[C@H](COC)C(=O)NCc1ccccc1]

In still other embodiments, the compound is one where -T is a covalently bonded antioxidant; a pro-drug to an antioxidant; or a precursor to an antioxidant. Examples of anti-oxidants which may be conjugated with huperzine or an analog thereof include, but are not limited to, ascorbic acid, glutathione, lipoic acid, uric acid, beta-carotene, vitamin A, vitamin E, co-enzyme Q; a pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (EGC), epicatechin gallate (ECG), and epigallocatechin gallate (EGCG), epigallocatechin and gallic acid, methyl gallate, and any combination thereof.

In yet other embodiments, the compound is one wherein -T is the following structure or pharmaceutically acceptable salt thereof bonded through any one of NH, NH₂, COOH, OH, thiol, or an enol-tautomer oxygen of one of the following compounds: uric acid, ascorbic acid, glutathione, melatonin, alpha-tocopherol, beta-tocopherol, gamma-tocopherol, delta-tocopherol, alpha-tocotrienols beta-tocotrienol, gamma-tocotrienol, delta-tocotrienol, catechin, epicatechin, epigallocatechin, epicatechin gallate, and epigallocatechin epigallocatechin, gallic acid, methyl gallate, or a combination thereof.

In some embodiments, a huperzine conjugate or huperzine analog conjugate is co-administered with an antioxidant. Examples of antioxidants which may be co-administered with huperzine or an analog thereof include, but are not limited to, ascorbic acid, glutathione, lipoic acid, uric acid, beta-carotene, vitamin A, vitamin E, co-enzyme Q; it pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (EGC), epicatechin gallate (ECG), and epigallocatechin gallate (EGCG), epigallocatechin and gallic acid, methyl gallate, and any combination thereof.

In still other embodiments, the compound is one where -T is a covalently bonded anti-inflammatory agent; a pro-drug to an anti-inflammatory agent; or a precursor to an anti-inflammatory agent. Examples of anti-inflammatory agents which may be conjugated with huperzine or an analog thereof include, but are not limited to, compounds of non-steroidal anti-inflammatory drugs, immune-selective anti-inflammatory derivatives, anti-inflammatory herbal extracts, extracts of salix purpurea, extracts of piper longum, extracts of boswellia serrata and extracts prunella vulgaris, NF-Kappa B inhibitors, IL-inhibitors, any combinations thereof, and/or the like. Additional examples of anti-inflammatory agents include, but are not limited to, tanshinone, cryptotanshinone, ferulic acid, cycloartenol, cycloartenyl ferulate, hydroxytyrosol, homovanillyl alcohol, 4-O-methylgallic acid, and any combination thereof.

In yet other embodiments, the compound is one wherein -T is the following structure or pharmaceutically acceptable salt thereof bonded through any one of COOH or OH of one of the following compounds: ferulic acid, cycloartenol, cycloartenyl ferulate, hydroxytyrosol, homovanillyl alcohol, 4-O-methylgallic acid, and any combination thereof.

In some embodiments, a huperzine conjugate or huperzine analog conjugate is co-administered with an anti-inflammatory agent. Examples of anti-inflammatory agents which may be co-administered with huperzine or an analog thereof include, but are not limited to, non-steroidal anti-inflammatory drugs, immune-selective anti-inflammatory derivatives, anti-inflammatory herbal extracts, NF-Kappa B inhibitors, IL-inhibitors, any combinations thereof, and/or the like. Additional examples of anti-inflammatory agents include, but are not limited to, tanshinone, cryptotanshinone, ferulic acid, cycloartenol, cycloartenyl ferulate, hydroxytyrosol, homovanillyl alcohol, extracts of salix purpurea, extracts of piper longum, 4-O-methylgallic acid, extracts of boswellia serrata and extracts of prunella vulgaris, and any combination thereof.

In some embodiments, the compound is one in which -T is a covalently bonded mitochondrial protectant selected from bethanechol and (2S,2′R,3′S,5′R)-1-methyl-2-(2-methyl-1,3-oxathiolan-5-yl)pyrrol-idine 3-sulfoxide methyl iodide; a pro-drug of the mitochondrial protectant, a precursor of the mitochondrial protectant; and any combination thereof. In various embodiments, -T is any one of the structures CXIX-CXX, or pharmaceutically acceptable salt thereof:

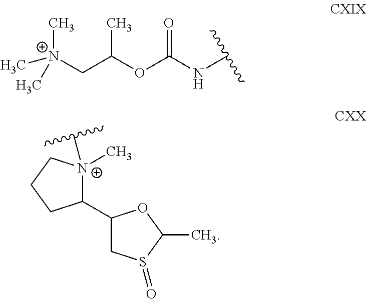 [SMILES: *NC(=O)OC(C)C[N+](C)(C)C ; *[N+]1(C)CCCC1C1CS(=O)C(C)O1]

In other embodiments, the compound is one in which -T is a covalently bonded anti-inflammatory agent selected from non-steroidal anti-inflammatory drugs, immune-selective anti-inflammatory derivatives, and anti-inflammatory herbal extracts, Kappa B inhibitors, and IL-inhibitors; a pro-drug of the anti-inflammatory agent, a precursor of the anti-inflammatory agent; and any combination thereof.

In some embodiments, -T is a covalently bonded alpha-7 agonist selected from 1,3,4-oxadiazol-2-amine, (+)—N-(1-azabicyclo[2.2.2]oct-3-yl)benzo[b]furan-carboxamide, AR-R17779, TC-5619, GTS-21, PHA-543,613, PNU-282,987, SSR-180,711, tropisetron, WAY-317,538, choline and nicotine (37-38); a pro-drug of the alpha-7agonist; a precursor of the alpha-7 agonist; and any combination thereof. In various embodiments, -T is any one of the structures XXI-XXV, XXVII-XXVIII, XXXII-XXXIV, XXXVII-XXXVIII, or pharmaceutically acceptable salt thereof:

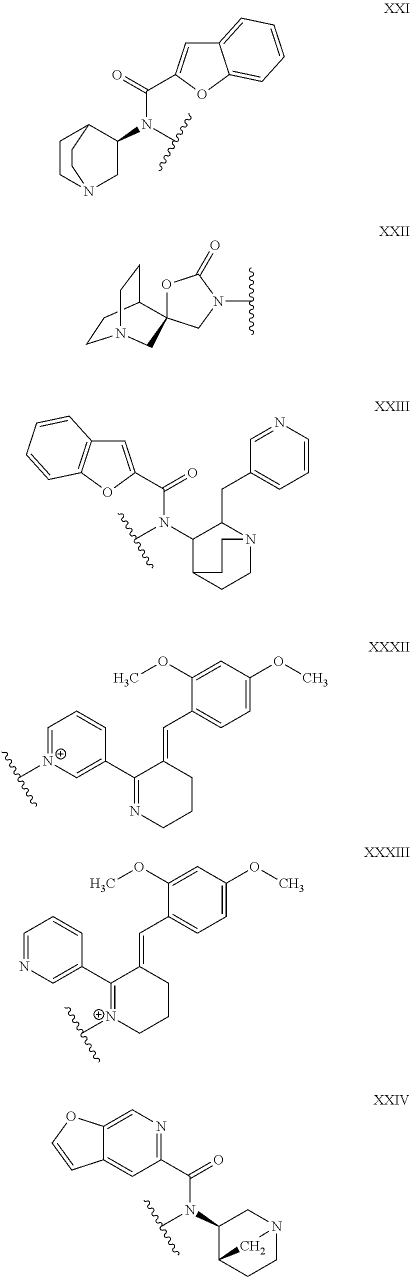 [SMILES: *N(C(=O)c1cc2ccccc2o1)[C@H]1CN2CCC1CC2 ; *N1C[C@@]2(CN3CCC2CC3)OC1=O ; *N(C(=O)c1cc2ccccc2o1)C1C2CCN(CC2)C1Cc1cccnc1 ; *[n+]1cccc(C2=NCCC/C2=C\c2ccc(OC)cc2OC)c1 ; *[N+]1=C(c2cccnc2)/C(=C/c2ccc(OC)cc2OC)CCC1 ; *N(C(=O)c1cc2ccoc2cn1)[C@H]1CN2CC[C@H]1C2]

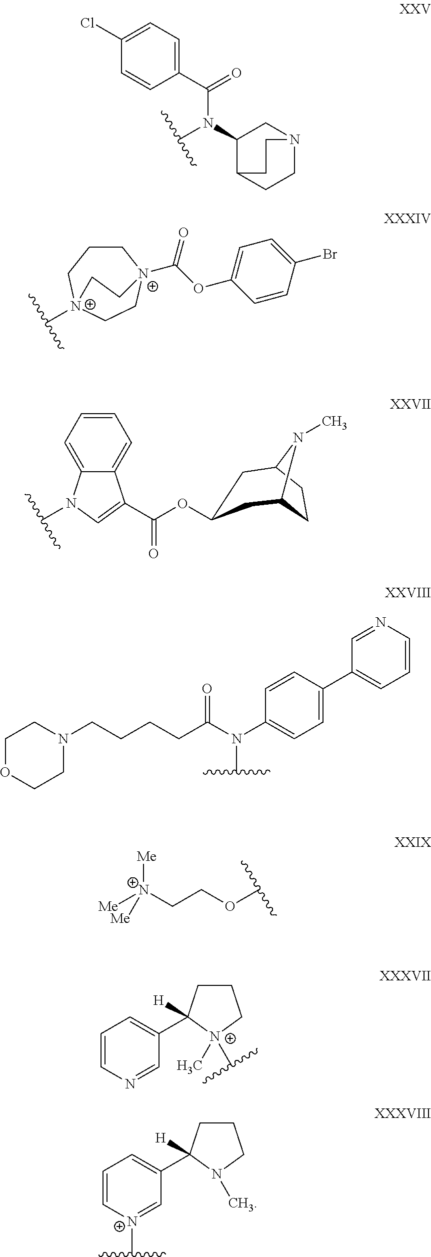

In some embodiments, the compound is one in which -T is a covalently bound potassium channel blocker selected from dofetilide, sotalol, ibutilide, azimilide, E-4031, nifekalant, tedisamil, sematilide, 4-aminopyridine, and 3,4-diaminopyridine; a pro-drug of the potassium channel blocker, a precursor of the potassium channel blocker; and any combination thereof. In various embodiments, -T is any one of the structures XVII-XIX, or pharmaceutically acceptable salt thereof.

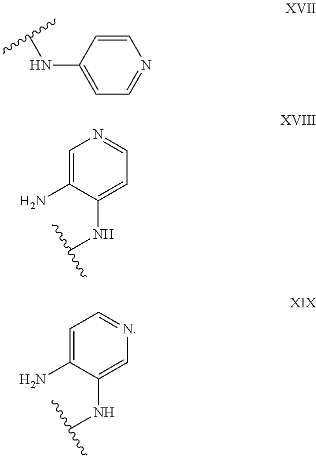

In some embodiments, -T is a sodium channel blocker selected from propranolol, procainamide, quinidine, disopyramide, lidocane, mexiletine, tocainide, phenytoin, encainide, flecainide, moricizine, propafenone, riluzole; a pro-drug of the sodium channel blocker; a precursor of the sodium channel blocker; and any combination thereof, in yet other embodiments, -T is an anticonvulsant selected from pregabalin, (S)-pregabalin, gabapentin, stiripentol, phenobarbital, methylphenobarbital, barbexaclone, lorazepam, nitrazepam, temazepam, nimetazepam, felbamate, carbamazepine, oxcarbazepine, eslicarbazepine acetate, valproic acid, vigabatrin, progabide, tiagabine, topiramate, ethotoin, phenytoin, mephenytoin, fosphenytoin, pheneturide, beclamide, primidone, brivaracetani, levetiraeetairt, seletracetam, ethosuximide, acetazolamide, suitiame, phenacemide, methazolamide, zonisamide, lamotrigine; a pro-drug of the anticonvulsant, a precursor of the anticonvulsant; and any combination thereof.

In still other embodiments, -T is an anxiolytic agent selected from positive allosteric modulators of GABA receptor, serotonin-specific re-uptake inhibitors (SSRI), barbiturates, and benzodiazepines; a pro-drug, of the anxiolytic agent, a precursor of the anxiolytic agent; and any combination thereof. In yet other embodiments, -T is an anxiolytic agent selected from carisoprodol, glutethimide, meprobamate, propofol, theanine, hydroxyzine, valcrenie acid, niacin, niacinamide; a pro-drug of the anxiolytic agent, a precursor of the anxiolytic agent; and any combination thereof.

In some embodiments, -T is an NADPH oxidase inhibitor agent selected from apocynin, a pro-drug of the NADPH oxidase inhibitor agent, a precursor of the NADPH oxidase inhibitor agent; and any combination thereof. In one embodiment, -T has the structure structures XV, or pharmaceutically acceptable salt thereof:

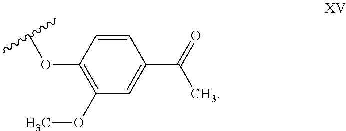 [SMILES: *Oc1ccc(C(C)=O)cc1OC]

In some embodiments of the compound, -T is a gamma amino butyric acid (GABA) reuptake inhibitor agent selected from nipecotic acid; a pro-drug of the GABA reuptake inhibitor agent, a precursor of the GABA reuptake inhibitor agent; and any combination thereof. In various embodiments, -T is any one of the structures XXXIX-XLI, or pharmaceutically acceptable salt thereof:

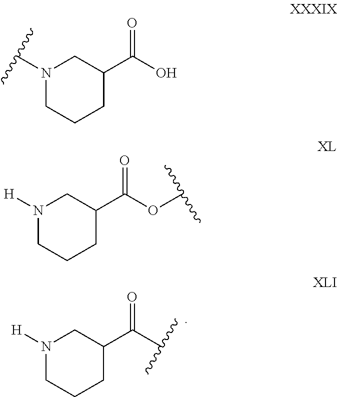 [SMILES: *N1CCCC(C(=O)O)C1 ; *OC(=O)C1CCCNC1 ; *C(=O)C1CCCNC1]

In some embodiments of the compound, -T is a monoamine oxidase B (MAO-B) inhibitor agent selected from lazabemide, pargyline, rasagiline, entacapone, tolcapone, nitecapone, and quercetin; a pro-drug of the MAO-B inhibitor agent, a precursor of the MAO-B inhibitor agent; and any combination thereof. In various embodiments, T is one of structures or pharmaceutically acceptable salt thereof:

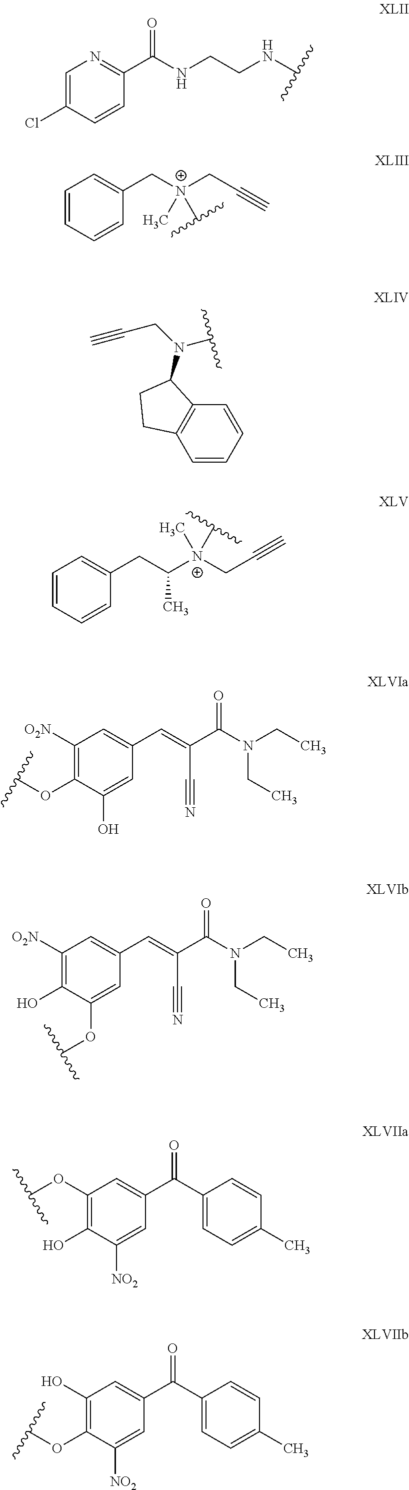

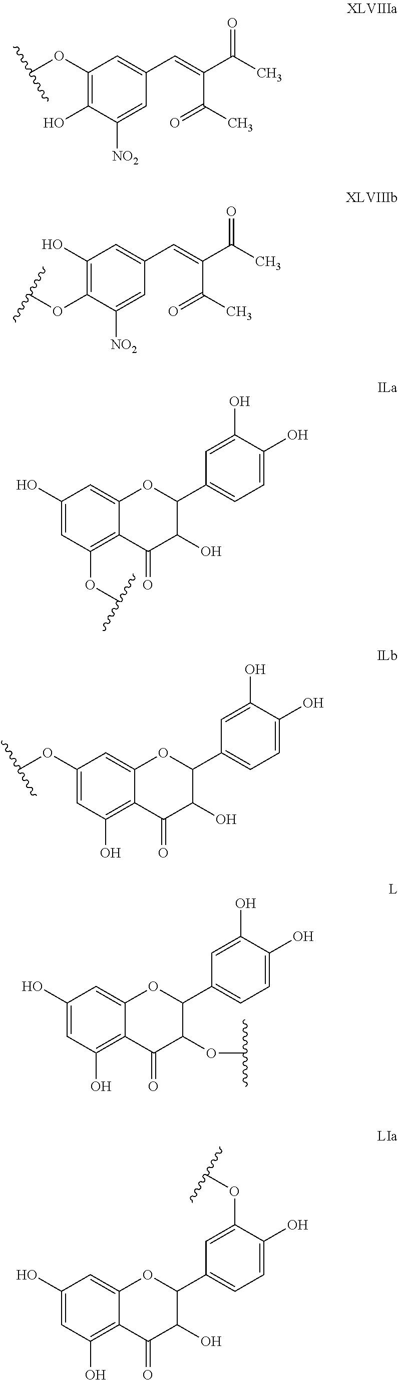 [SMILES: *Oc1cc(C=C(C(C)=O)C(C)=O)cc([N+](=O)[O-])c1O ; *Oc1c(O)cc(C=C(C(C)=O)C(C)=O)cc1[N+](=O)[O-] ; *Oc1cc(O)cc2c1C(=O)C(O)C(c1ccc(O)c(O)c1)O2 ; *Oc1cc(O)c2c(c1)OC(c1ccc(O)c(O)c1)C(O)C2=O ; *OC1C(=O)c2c(O)cc(O)cc2OC1c1ccc(O)c(O)c1 ; *Oc1cc(C2Oc3cc(O)cc(O)c3C(=O)C2O)ccc1O]

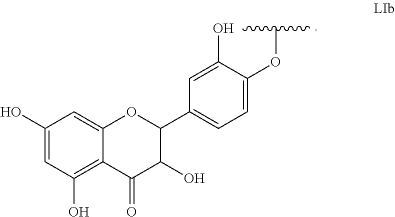 [SMILES: *Oc1ccc(C2Oc3cc(O)cc(O)c3C(=O)C2O)cc1O]

In some embodiments of the compound, -T is muscarinic receptor antagonist agent selected from atropine, cycycloverine, diphenhydramine, tolterodine, oxybutynin, opratropium, chlorpormazine, methoctramine, tripitramine, and gallamine; a pro-drug of the muscarinic receptor antagonist, a precursor of the muscarinic receptor antagonist; and any combination thereof. In various embodiments, -T is any one of structures LII-LXIII, or pharmaceutically acceptable salt thereof:

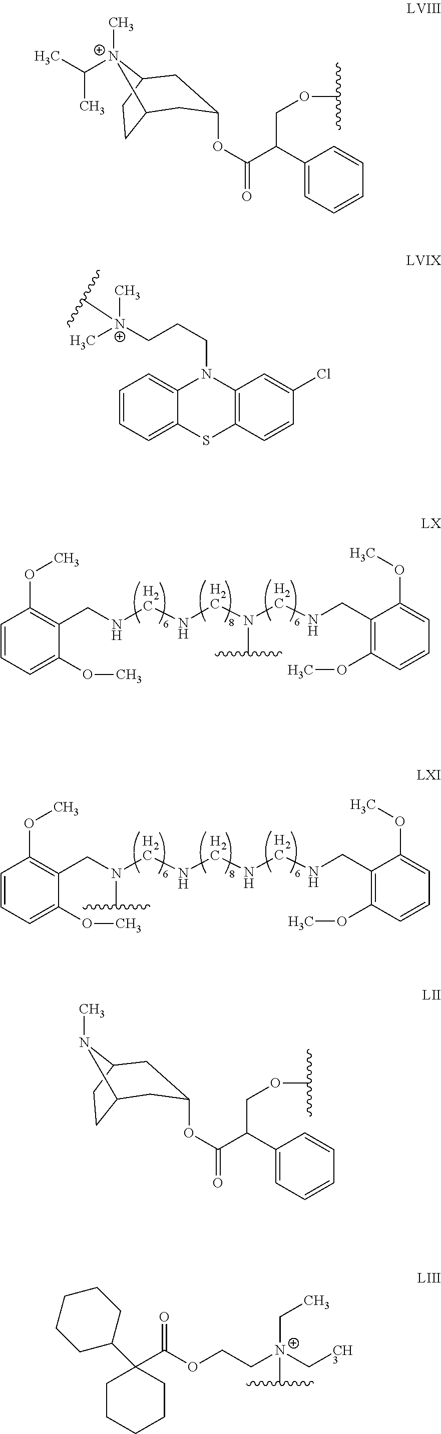

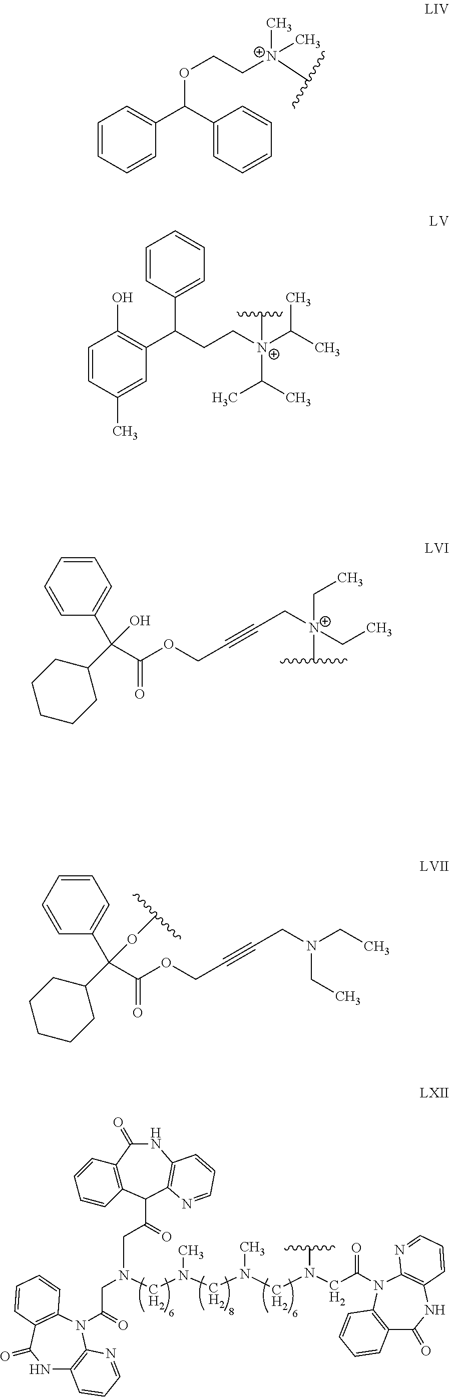

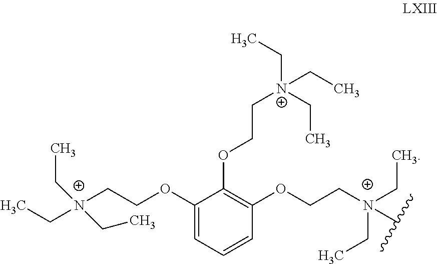 [SMILES: *[N+](CC)(CC)CCOc1cccc(OCC[N+](CC)(CC)CC)c1OCC[N+](CC)(CC)CC]

In some embodiments of the compound, -T is a dopamine receptor antagonist agent selected from malperone, risperidone, ziprasidone, raclopride, clozapine, haloperidol, quetiapine, domperidone, eticlopride, yohimbine, blonanserin and L-741,626 (3-[4-(4-Chlorophenyl)-4-hydroxypiperidin-1-yl]methyl-1H-indole); a pro-drug of the dopamine receptor antagonist, a precursor of the dopamine receptor antagonist, and any combination thereof. In various embodiments, -T is any one of structures LIX-LXXIII, or pharmaceutically acceptable salt thereof.

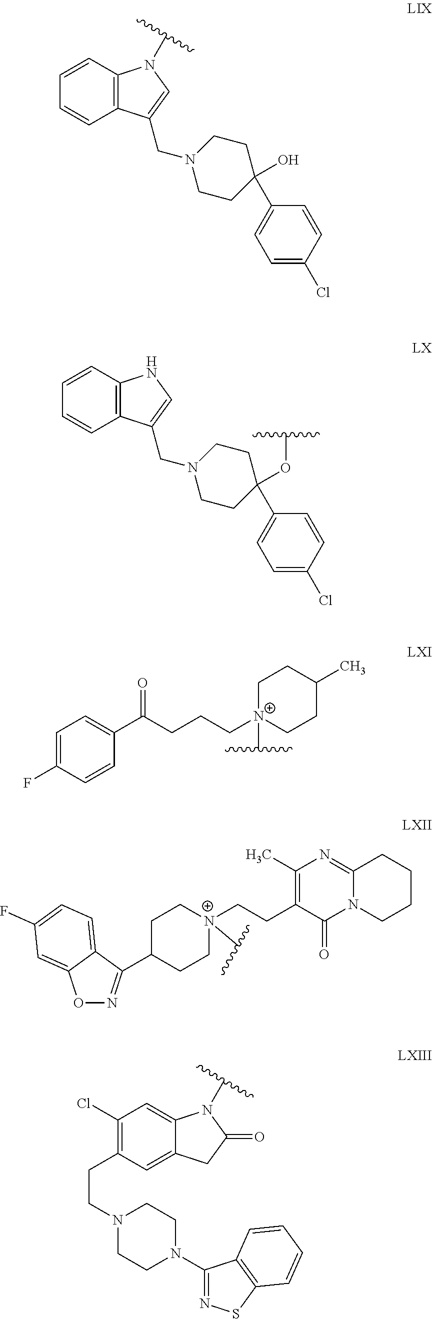 [SMILES: *n1cc(CN2CCC(O)(c3ccc(Cl)cc3)CC2)c2ccccc21 ; *OC1(c2ccc(Cl)cc2)CCN(Cc2c[nH]c3ccccc23)CC1 ; *[N+]1(CCCC(=O)c2ccc(F)cc2)CCC(C)CC1 ; *[N+]1(CCc2c(C)nc3n(c2=O)CCCC3)CCC(c2noc3cc(F)ccc23)CC1 ; *N1C(=O)Cc2cc(CCN3CCN(c4nsc5ccccc45)CC3)c(Cl)cc21]

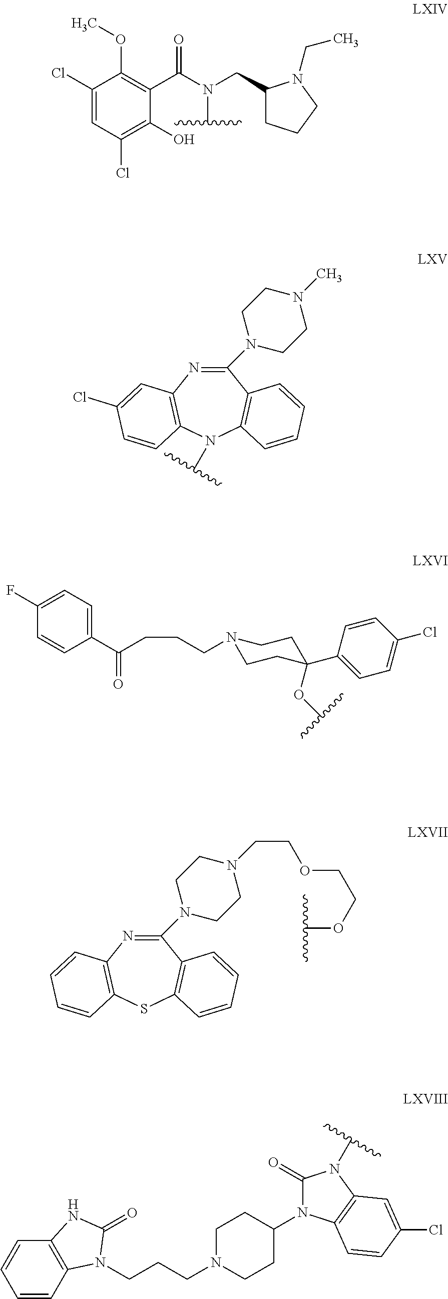

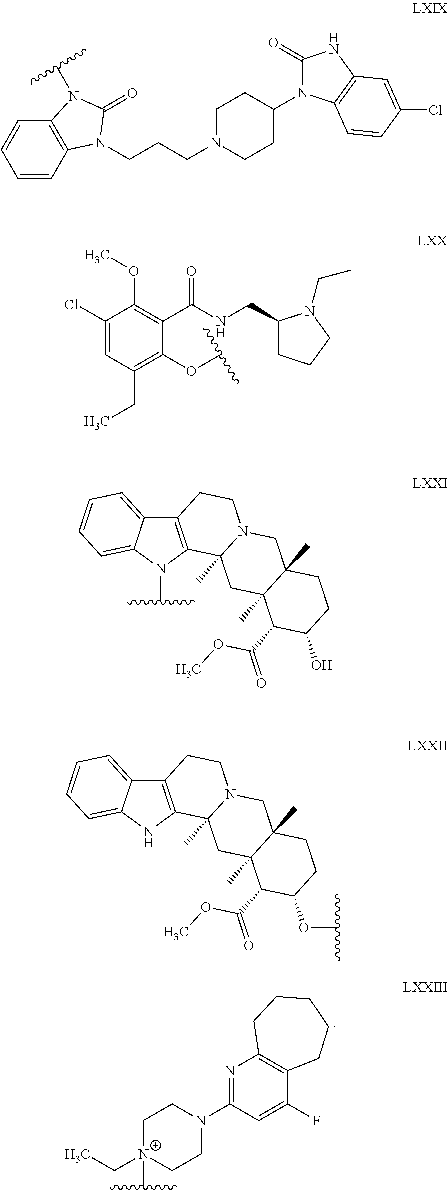

In some embodiments of the compound, -T is a glutamate receptor (NR2B) antagonist agent selected from ifenprodil; a prodrug of the NR2B antagonist, a precursor of the NR2B antagonist, and any combination thereof. In various embodiments, -T is any one of structures LXXIV-LXXV, or pharmaceutically acceptable salt thereof:

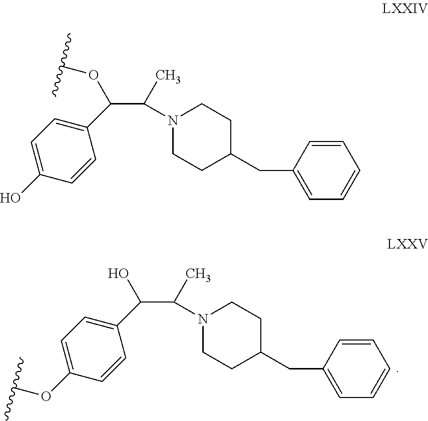 [SMILES: *OC(c1ccc(O)cc1)C(C)N1CCC(Cc2ccccc2)CC1 ; *Oc1ccc(C(O)C(C)N2CCC(Cc3ccccc3)CC2)cc1]

In some embodiments of the compound, -T is epigallocatechin gallate (EGCG); a pro-drug of EOM a precursor of EGCG, and any combination thereof. In various embodiments, -T is any one of structures LXXVIa-LXVIII, or pharmaceutically acceptable salt thereof:

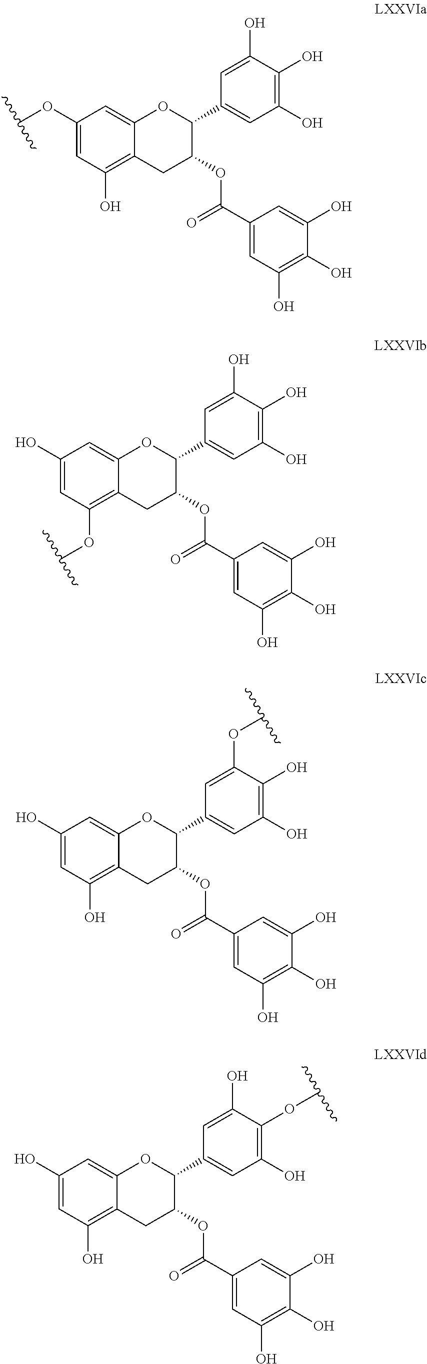 [SMILES: *Oc1cc(O)c2c(c1)O[C@H](c1cc(O)c(O)c(O)c1)[C@H](OC(=O)c1cc(O)c(O)c(O)c1)C2 ; *Oc1cc(O)cc2c1C[C@@H](OC(=O)c1cc(O)c(O)c(O)c1)[C@@H](c1cc(O)c(O)c(O)c1)O2 ; *Oc1cc([C@H]2Oc3cc(O)cc(O)c3C[C@H]2OC(=O)c2cc(O)c(O)c(O)c2)cc(O)c1O ; *Oc1c(O)cc([C@H]2Oc3cc(O)cc(O)c3C[C@H]2OC(=O)c2cc(O)c(O)c(O)c2)cc1O]

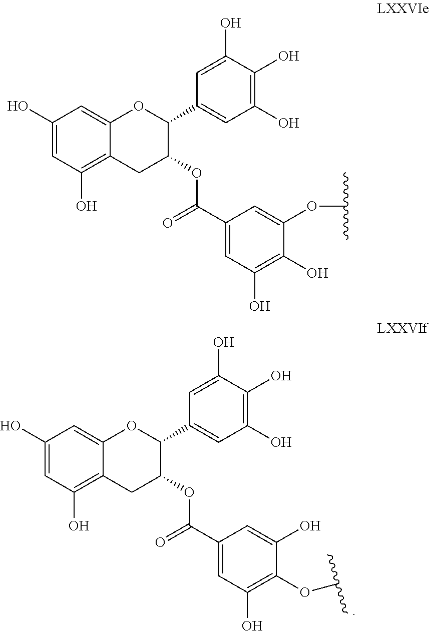 [SMILES: *Oc1cc(C(=O)O[C@@H]2Cc3c(O)cc(O)cc3O[C@@H]2c2cc(O)c(O)c(O)c2)cc(O)c1O ; *Oc1c(O)cc(C(=O)O[C@@H]2Cc3c(O)cc(O)cc3O[C@@H]2c2cc(O)c(O)c(O)c2)cc1O]

In some embodiments of the compound, -T is an aromatase inhibitor agent selected from aminoglutethimide and formestane; a prodrug of the aromatase, inhibitor, a precursor of the aromatase inhibitor, and any combination thereof. In various embodiments, −T is any one of structures LXVII-LXXVIII, or pharmaceutically acceptable salt thereof:

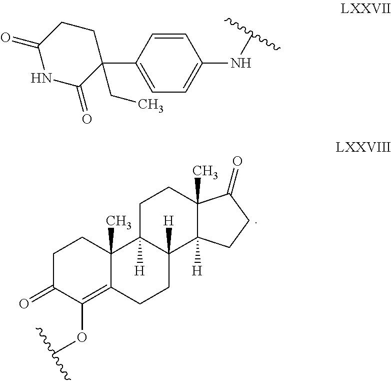

In some embodiments of the compound, -T is one of the following structures:

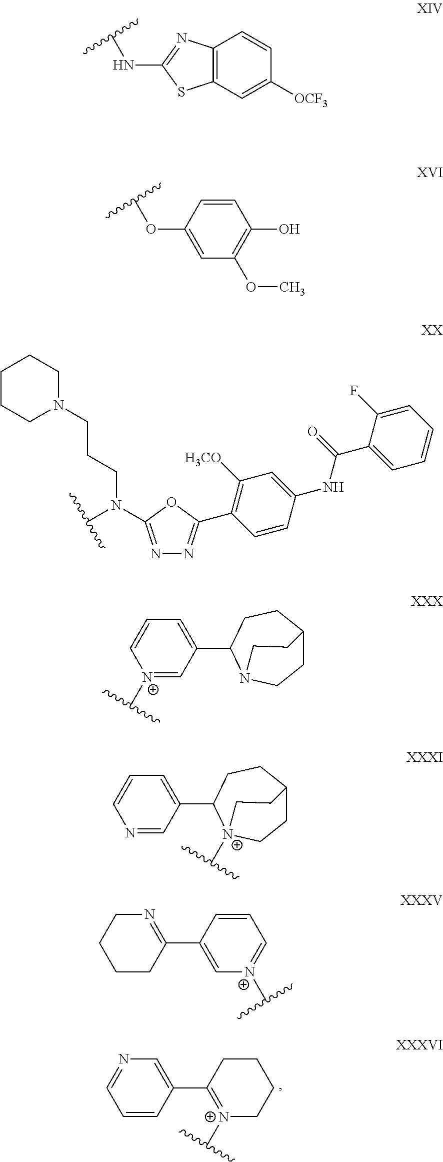

or pharmaceutically acceptable salt thereof

In an embodiment, the compound has the structure:

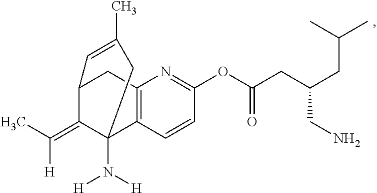 [SMILES: C/C=C1\C2C=C(C)CC1(N)c1ccc(OC(=O)C[C@@H](CN)CC(C)C)nc1C2]

or pharmaceutically acceptable salt thereof

In another embodiment, the compound has the structure:

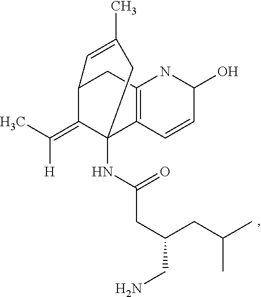 [SMILES: C/C=C1\C2C=C(C)CC1(NC(=O)C[C@@H](CN)CC(C)C)C1=C(C2)NC(O)C=C1]

or pharmaceutically acceptable salt thereof

Yet another embodiment, the compound has the structure:

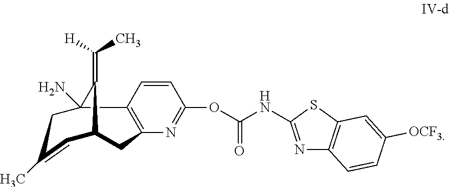

or pharmaceutically acceptable salt thereof.

In still another embodiment, the compound has the structure:

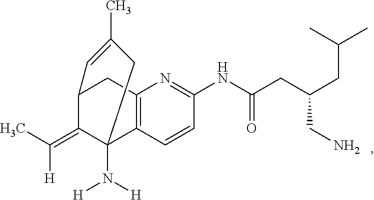 [SMILES: C/C=C1\C2C=C(C)CC1(N)c1ccc(NC(=O)C[C@@H](CN)CC(C)C)nc1C2]

or pharmaceutically acceptable salt thereof.

In another embodiment, the compound has the structure:

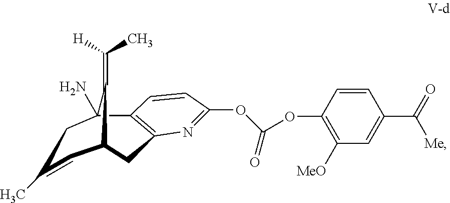

or pharmaceutically acceptable salt thereof,

In another embodiment, the compound has the following structure:

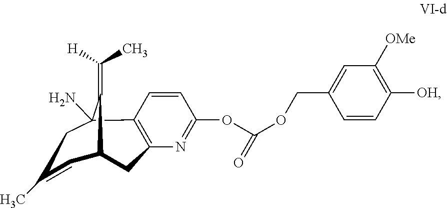

or pharmaceutically acceptable salt thereof

In still another embodiment, the compound has a structure:

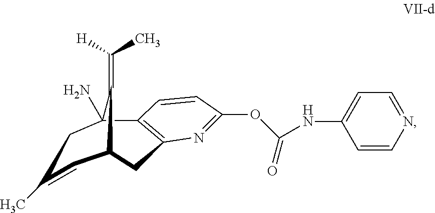

or pharmaceutically acceptable salt thereof.

In yet another embodiment, the compound has the structure:

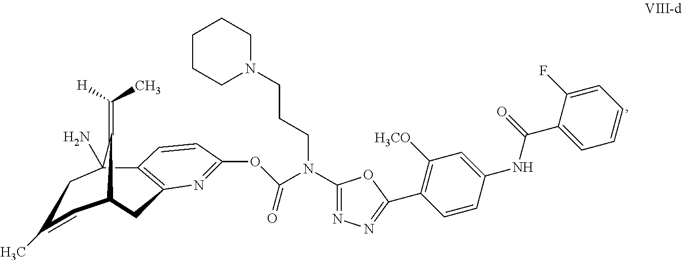

or pharmaceutically acceptable salt thereof

In still another embodiment, the compound has the structure:

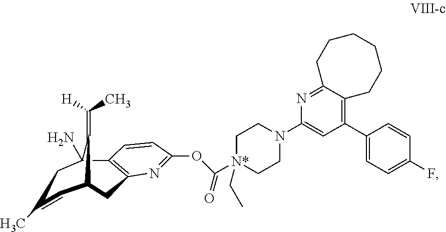

or pharmaceutically acceptable salt thereof.

In yet another embodiment, the compound has a structure:

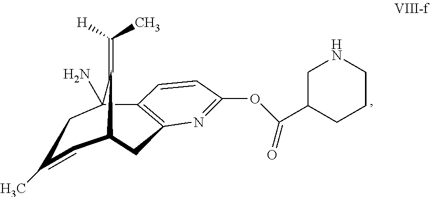

or pharmaceutically acceptable salt thereof.

In an embodiment, the compound has the structure:

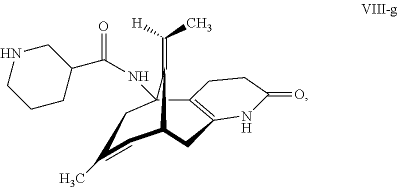

or pharmaceutically acceptable salt thereof.

In another embodiment, the compound has the structure:

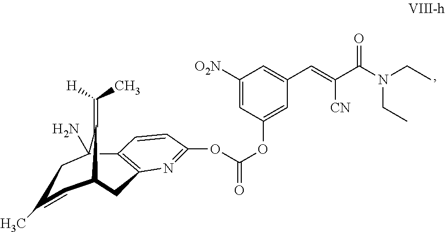

or pharmaceutically acceptable salt thereof.

In still another embodiment, the compound has the structure:

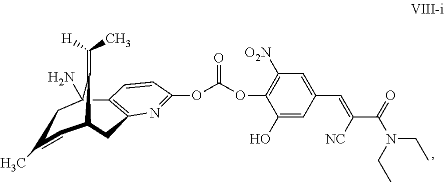

or pharmaceutically acceptable salt thereof.

In some aspects, the present disclosure is directed to methods of treating a neurodegenerative disease by administering a conjugate of a huperzine or huperzine analog, wherein the neurodegenerative disease is treated. In some embodiments, a therapeutically effective amount of the conjugate is administered.

An embodiment of the method for treating a neurodegenerative disease includes administering a therapeutically effective amount of a conjugate of huperzine or an analog of huperzine having the general formula:

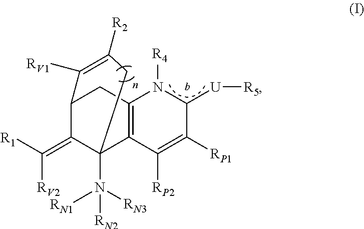

tautomer thereof, or pharmaceutically acceptable salt thereof, wherein R₁ is selected from H, (C₁-C₂₄)alkyl, CF₃, CF₂CF₃, CF₂CF₂CF₃, SO₂CH₃, SO₂Ph, SO₂Ar, SO₃H, and SO₃Ar, and —CH₂-L-T; R₂ is selected from H, (C₁-C₂₄)alkyl, aryl, cycloalkyl, (C₂-C₂₄)alkenyl, heterocycle, heteroaryl and —CH₂-L-T; R_{P1}, R_{P2}, R_{V1}, R_{V2} are independently selected from hydrogen and fluorine; R_{N1} is selected from H, (C₁-C₂₄)alkyl, CF₃, CF₂CF₃, CCl₃, CBr₃, CHO, and -L-T; R_{N1} and R_{N2} is selected from H, (C₁-C₂₄)alkyl, CF₃, CF₂CF₃, CCl₃, CBr₃, and CHO; R_{N3} is selected from absent and (C₁-C₂₄)alkyl; t is O, S, NH, or N((C₁-C₂₄)alkyl); b is a keto-enol tautomer unsaturation: and a is an integer selected from 1, 2, 3, and 4; R₄ is absent, or selected from H, and L-T; R₅ is absent, or selected from H, and -L-T; at least one of R₁, R₂, R_{N1}, R₄ and R₅ is -L-T; only one of R_{N1}, R₄ and is -L-T; each -L- is independently a linker that is selected from a bond, —O—, —S—, —NH—, —N(alkyl)-, —O(C═O)—, —C(═S)O—, —C(═S)—, and —P(O)₂—; and each -T is independently selected from a therapeutic agent, a therapeutic agent pro-drug, or a therapeutic agent precursor, wherein the neurodegenerative disease is treated. In embodiments, n=1, and R₁═R₂═CH₃.

In some embodiments of the method, the conjugate has R_{P1} is H or F; R_{P2} is H or F; R_{V1} is H or F; R_{V2} is H or F; wherein at least one of R_{P1}, R_{P2}, R_{V1}, and R_{V2} is fluorine. In another embodiment of the method, the conjugate has R_{P1} is H or F; R_{P2} is H or F; R_{V1} is H or F; R_{V2} is H or F; wherein at least one of R_{P1}, R_{P2}, and R_{V1} is fluorine; R₁ is methyl; R₂ is methyl; R_{N1} is H; R_{N2} is H; and R_{N3} is absent.

In some embodiments of the method for treating a neurodegenerative disease includes administering a therapeutically effective amount of a conjugate wherein the linker may comprise at least one of a linker (L) functional group selected from a bond, —O—, —S—, —NH—, —N(alkyl)-, —C(O)—, —O(C═O)—, —C(═O)O—, —C(═S)O—, —C(═S)—, and —P(O)₂—, wherein the neurodegenerative disease is treated. In various embodiments, the linker is a bond. In other embodiments, the linker is —C(O)—. In other embodiments, the linker is —C(═S)—. In still other embodiments, the linker is —P(O)₂—. In yet other embodiments, the linker is an ether, sulfide, or an amine. In some embodiments, the linker is —O(C═O)—, or in other embodiments, —C(═O)O—.

In some embodiments of the method, the conjugate administered has each -T therapeutic agent, therapeutic agent pro-drug, or therapeutic agent precursor is independently —V—W—X—Y—Z, wherein V is bond, —O—, or —NH—; W is —(C₀-C₆)alkyl-, —(C₂-C₆)alkenyl-; or —(C₂-C₆)alkynyl-; X is a bond, —O—, —NH—, —CO—, —(C═O)NH—, —NH—(C═O)—, —SO₂—, —(C═NH)—NH—, —(C═O)—O—, or —O(C═O)—; Y is a —(C₀-C₆)alkyl-, —(C₂-C₆)alkenyl-; or —(C₂-C₆)alkynyl-; Z is a -quaternary amine, -cycloalkyl, -aryl, -heterocycle, or heteroaryl; and each nitrogen, alkyl, alkenyl, alkynyl, cycloalkyl, aryl, heterocycle, or heteroaryl is optionally substituted, wherein the neurodegenerative disease is treated.

In still other embodiments of the method for treating the neurodegenerative disease is administering a therapeutically effective amount of the huperzine or huperzine analog conjugate of formula I, wherein -T is a covalently bonded glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor; a precursor to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor, a gamma amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof, wherein the neurodegenerative disease is treated.

In still other embodiments of the method for treating the neurodegenerative disease is administering a therapeutically effective amount of the huperzine or huperzine analog conjugate of formula I, wherein -T is —(C═O)—CH₂—CH(i-Bu)-CH₂—NH₂, wherein the neurodegenerative disease is treated. In other embodiments, -T is (S)—(C═O)—CH₂—CH(i-Bu)-CH₂—NH₂.

In yet other embodiments of the method for treating the neurodegenerative disease is administering a therapeutically effective amount of the huperzine or huperzine analog conjugate of formula I, wherein -T is a covalently bonded N-methyl d-aspartate receptor antagonist selected from R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantadine, dextrallorphan, dextromethorphan, dextrorphan, dizocilpine, eticyclidine, gacyclidine, ibogaine, memantine, methoxetamine, phencyclidine, rolicyclidine, tenocyclidine, methoxydine, tiletamine, neramexane, eliprodil, etoxadrol, dexoxadrol, remacemide, delucemine, Sa-phenyldecahydroquinoline, aptiganel, rhynchophylline, ketamine, 1-aminocyclopropanecarboxylic acid, 7-chlorokynurenate, 5,7-dichlorokynureinc acid, kynurenic acid, lacosamide; a pro-drug of the NMDA receptor antagonist, a precursor of the NMDA receptor antagonist; and any combination thereof.

In still other embodiments of the method for treating the neurodegenerative disease is administering a therapeutically effective amount of the huperzine or huperzine analog conjugate of formula I, wherein the compound is one wherein -T is any one of the structures XIXXIX-CXVIII, or pharmaceutically acceptable salt thereof:

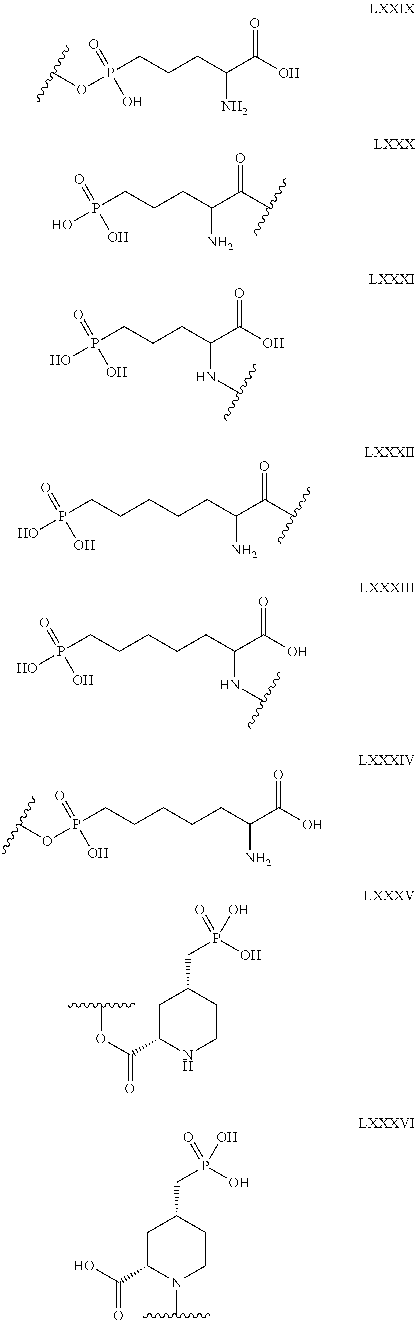

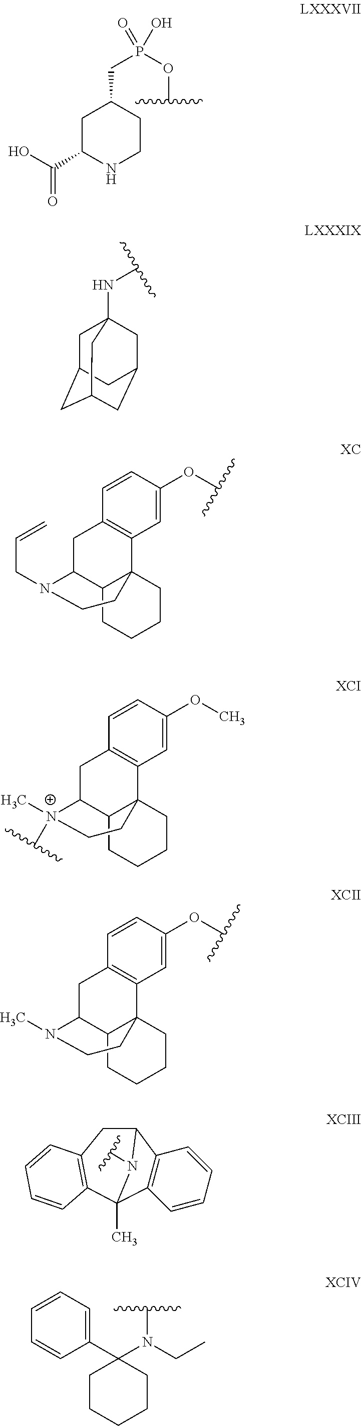

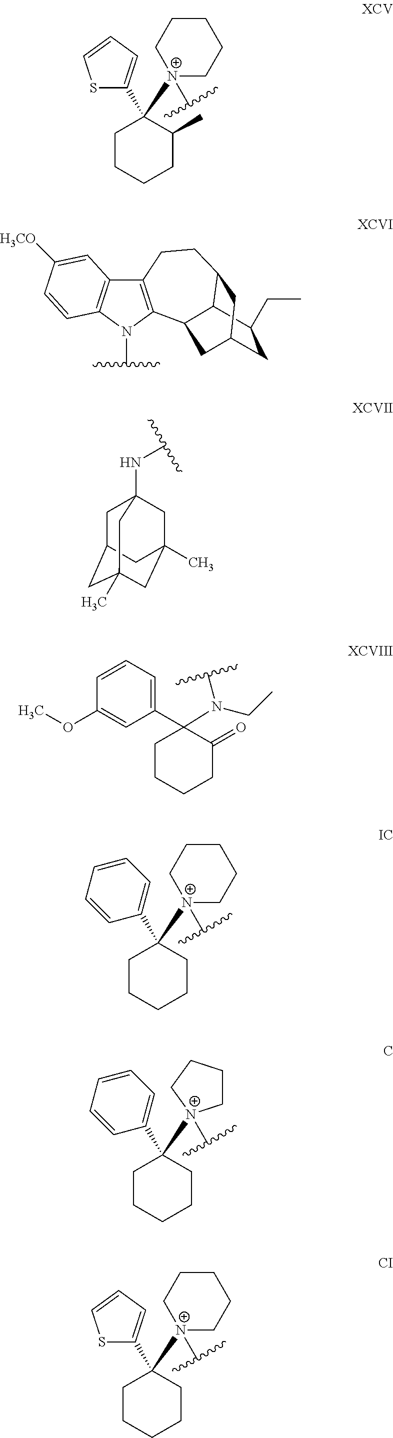

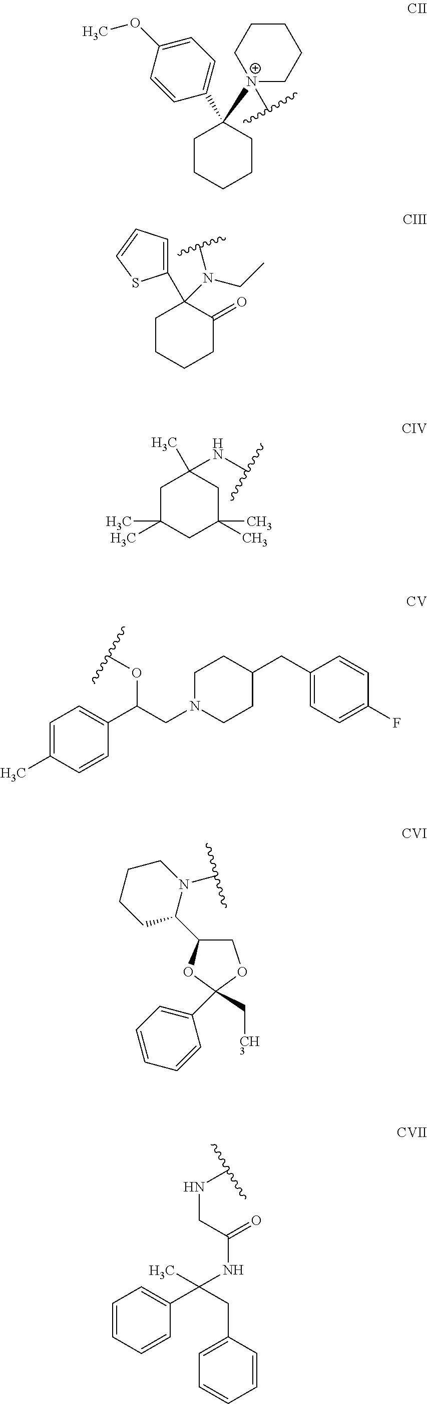

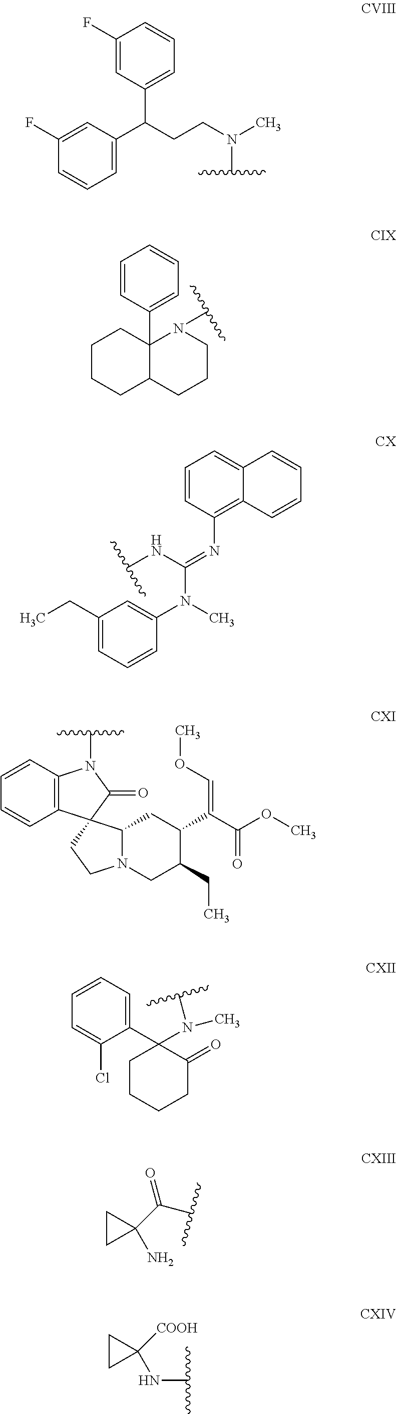

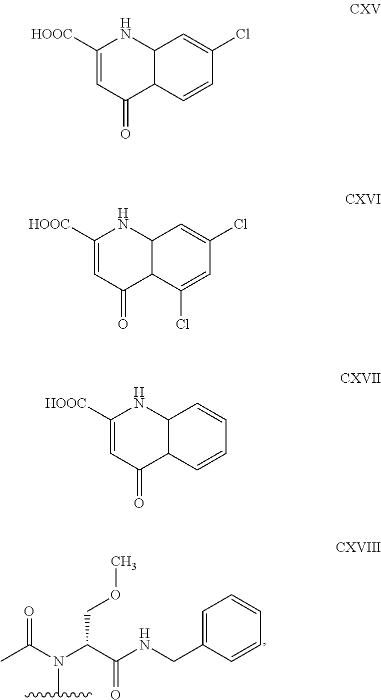 [SMILES: O=C(O)C1=CC(=O)C2C=CC(Cl)=CC2N1 ; O=C(O)C1=CC(=O)C2C(Cl)=CC(Cl)=CC2N1 ; O=C(O)C1=CC(=O)C2C=CC=CC2N1 ; *N(C(C)=O)[C@H](COC)C(=O)NCc1ccccc1]

wherein the neurodegenerative disease is treated.

In some embodiments of the method for treating the neurodegenerative disease is administering a therapeutically effective amount of the huperzine or huperzine analog conjugate of formula I, wherein the compound is one in which -T is a covalently bonded mitochondrial protectant selected from bethanechol and (2S,2′R,3′S,5′R)-1-methyl-2-(2-methyl-1,3-oxathiolan-5-yl)pyrrol-idine 3-sulfoxide methyl iodide; a pro-drug of the mitochondrial protectant, a precursor of the mitochondrial protectant; and any combination thereof, wherein the neurodegenerative disease is treated. In various embodiments, -T is any one of the structures CXIX-CXX, or pharmaceutically acceptable salt thereof:

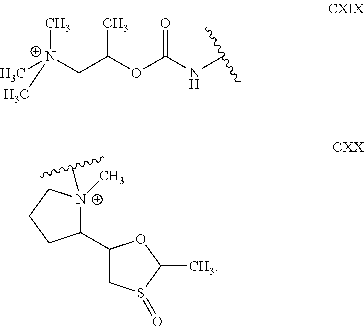 [SMILES: *NC(=O)OC(C)C[N+](C)(C)C ; *[N+]1(C)CCCC1C1CS(=O)C(C)O1]

In still other embodiments of the method, the compound is one where is a covalently bonded antioxidant; a pro-drug to an antioxidant; or a precursor to an antioxidant. Examples of anti-oxidants which may be conjugated with huperzine or an analog thereof include, but are not limited to, ascorbic acid, glutathione, lipoic acid, uric acid, beta-carotene, vitamin A, vitamin E, co-enzyme Q; a pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (EGC), epicatechin gallate (ECG), and epigallocatechin gallate (EGCG), epigallocatechin, gallic acid, methyl gallate, and any combination thereof.

In yet other embodiments of the method, the compound is one wherein -T is the following structure or pharmaceutically acceptable salt thereof bonded through any one of NH, NH₂, COOH, OH, thiol, or an enol-tautomer oxygen of one of the following compounds: uric acid, ascorbic acid, glutathione, melatonin, alpha-tocopherol, beta-tocopherol, gamma-tocopherol, delta-tocopherol, alpha-tocotrienols beta-tocotrienol, gamma-tocotrienol, delta-tocotrienol, tocotrienol, catechin, epicatechin, epigallocatechin, epicatechin gallate, and epigallocatechin gallate, epigallocatechin, gallic acid, methyl gallate, or a combination thereof.

In still other embodiments of the method, the compound is one where -T is a covalently bonded anti-inflammatory agent; a pro-drug to an anti-inflammatory agent; or a precursor to an anti-inflammatory agent. Examples of anti-inflammatory agents which may be conjugated with huperzine or an analog thereof include, but are not limited to, compounds of non-steroidal anti-inflammatory drugs, immune-selective anti-inflammatory derivatives, anti-inflammatory herbal extracts, extracts of salix purpurea, extracts of piper longum, extracts of boswellia serrata and extracts of prunella vulgaris, NT-Kappa B inhibitors, IL-inhibitors, any combinations thereof, and/or the like. Additional examples of anti-inflammatory agents include, but are not limited to, tanshinone, cryptotanshinone, ferulic acid, cycloartenyl, cycloartenyl ferulate, hydroxytyrosol, homovanillyl alcohol, 4-O-methylgallic acid, and any combination thereof.

In yet other embodiments of the method, the compound is one wherein -T is the followings structure or pharmaceutically acceptable salt thereof bonded through any one of COOH or OH of one of the following compounds: ferulic acid, cycloartenol, cycloartenyl ferulate, hydroxytyrosol, homovanillyl alcohol, 4-O-methylgallic acid, and any combination thereof.

In other embodiments of the method for treating the neurodegenerative disease is administering a therapeutically effective amount of the huperzine or huperzine analog conjugate of formula I, wherein the compound is one in which -T is a covalently bonded anti-inflammatory agent selected from non-steroidal anti-inflammatory drugs, immune-selective anti-inflammatory derivatives, and anti-inflammatory herbal extracts. NF-Kappa B inhibitors, and IL-inhibitors; a pro-drug of the anti-inflammatory agent, a precursor of the anti-inflammatory agent; and any combination thereof, wherein the neurodegenerative disease is treated.

In some embodiments of the method for treating the neurodegenerative disease is administering a therapeutically effective amount of the huperzine or huperzine analog conjugate of formula I, wherein -T is a covalently bonded alpha-7 agonist selected from 1,3,4-oxadiazol-2-amine, (+)—N-(1-azabicyclo[2.2.2]oct-3-yl)benzo[b]furan-2-carboxamide, AR-R17779, TC-5619, GTS-21, PHA-543,613, PNU-282,987, SSR-180,711, tropisetron, WAY-317,538, choline, and nicotine (37-38); a pro-drug of the alpha-7 agonist; a precursor of the alpha-7 agonist; and any combination thereof, wherein the neurodegenerative disease is treated. In various embodiments, -T is any one of the structures XXI-XXV, XXVII-XXVIII, XXXII-XXXIV, XXXVII-XXXVIII, or pharmaceutically acceptable salt thereof:

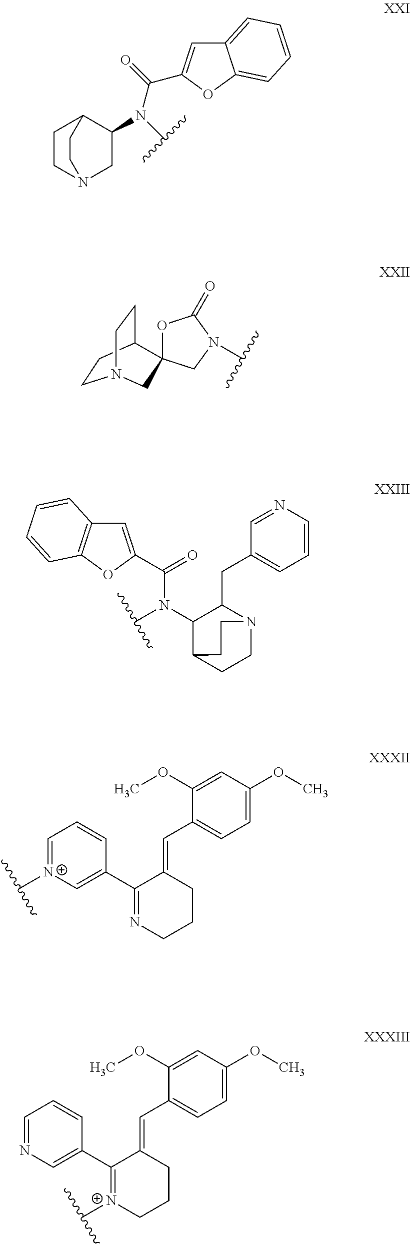 [SMILES: *N(C(=O)c1cc2ccccc2o1)[C@H]1CN2CCC1CC2 ; *N1C[C@@]2(CN3CCC2CC3)OC1=O ; *N(C(=O)c1cc2ccccc2o1)C1C2CCN(CC2)C1Cc1cccnc1 ; *[n+]1cccc(C2=NCCC/C2=C\c2ccc(OC)cc2OC)c1 ; *[N+]1=C(c2cccnc2)/C(=C/c2ccc(OC)cc2OC)CCC1]

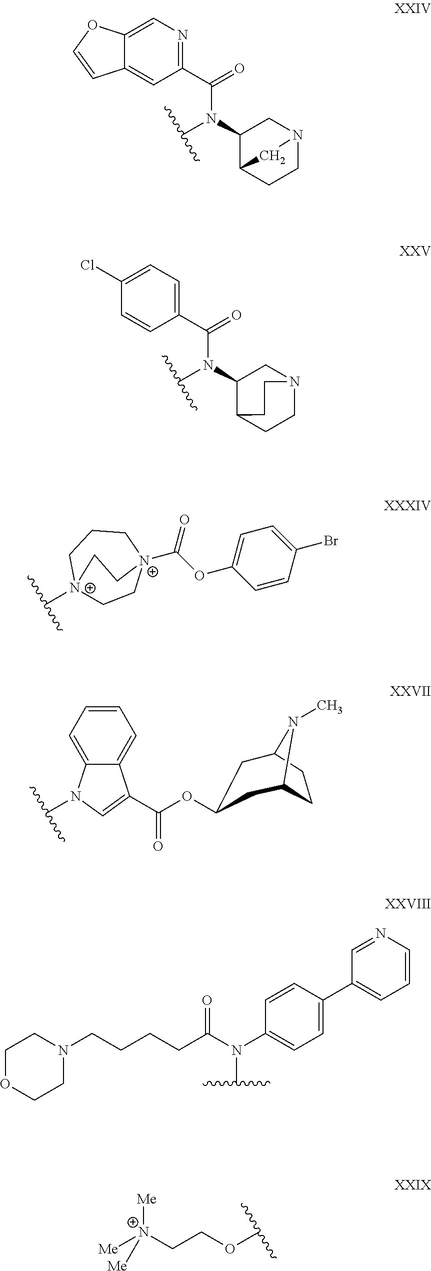

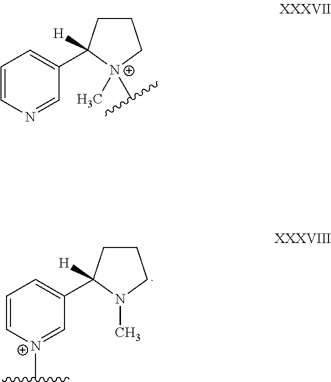 [SMILES: *[N+]1(C)CCC[C@H]1c1cccnc1 ; *[n+]1cccc([C@@H]2CCCN2C)c1]

In some embodiments of the method for treating the neurodegenerative disease is administering a therapeutically effective amount of the huperzine or huperzine analog conjugate of formula I, wherein the compound is one in which T is a covalently bound potassium channel blocker selected from dofetilide, sotalol, ibutilide, azimilide, E-4031, nifekalant, tedisamil, sematilide, 4-aminopyridine, and 3,4-diaminopyridine; a pro-drug of the potassium channel blocker; a precursor of the potassium channel blocker; and any combination thereof, wherein the neurodegenerative disease is treated. In various embodiments, -T is any one of the structures XVII-XIX, or pharmaceutically acceptable salt thereof:

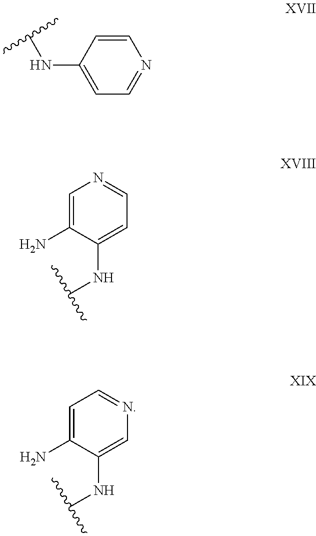 [SMILES: *Nc1ccncc1 ; *Nc1ccncc1N ; *Nc1cnccc1N]

In still other embodiments of the method for treating the neurodegenerative disease is administering a therapeutically effective amount of the huperzine or huperzine analog conjugate of formula I, wherein -T is a sodium channel blocker selected from propranolol, procainamide, quinidine, disopyramide, lidocane, mexiletine, tocainide, phenytoin, encainide, flecainide, moricizine, propafenone, riluzole; a pro-drug of the sodium channel blocker; a precursor of the sodium channel blocker; and any combination thereof, wherein the neuro degenerative disease is treated. In yet other embodiments, -T is an anticonvulsant selected from pregabalin, (S)-pregabalin, gabapentin, stiripentol, phenobarbital, methylphenobarbital, barbexaclone, lorazepam, nitrazepam, temazepam, nimetazepam, felbamate, carbamazepine, oxcarbazepine, eslicarbazepine acetate, valproic acid, vigabatrin, progabide, tiagabine, topiramate, ethotoin, phenytoin, mephenytoin, fosphenytoin, pheneturide, beclamide, primidone, brivaracetam, levetiracetam, seletracetam, ethosuximide, acetazolamide, sultiame, phenacemide, methazolamide, zonisamide, lamotrigine; a pro-drug of the anticonvulsant, a precursor of the anticonvulsant; and any combination thereof.

In still other embodiments of the method for treating the neurodegenerative disease is administering a therapeutically effective amount of the huperzine or huperzine analog conjugate of formula I, wherein -T is an anxiolytic agent selected from positive allosteric modulators of GABA receptor, serotonin-specific re-uptake inhibitors (SSRI), barbiturates, and benzodiazepines; a pro-drug of the anxiolytic agent, a precursor of the anxiolytic agent; and any combination thereof, wherein the neurodegenerative disease is treated. In yet other embodiments, -T is an anxiolytic agent selected from carisoprodol, glutethimide, meprobamate, propofol, theanine, hydroxyzine, valerenic acid, niacin, niacinamide; a pro-drug of the anxiolytic agent, a precursor of the anxiolytic agent; and any combination thereof.

In some embodiments of the method for treating the neurodegenerative disease is administering a therapeutically effective amount of the huperzine or huperzine analog conjugate of formula I, wherein -T is an NADPH oxidase inhibitor agent selected from apocynin, a pro-drug of the NADPH oxidase inhibitor agent, a precursor of the NADPH oxidase inhibitor agent; and any combination thereof, wherein the neurodegenerative disease is treated. In one embodiment, -T has the structure structures XV, or pharmaceutically acceptable salt thereof:

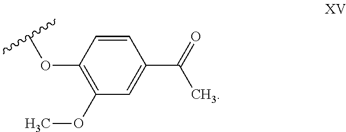 [SMILES: *Oc1ccc(C(C)=O)cc1OC]

In still other embodiments of the method for treating the neurodegenerative disease is administering a therapeutically effective amount of the huperzine or huperzine analog conjugate of formula I, wherein -T is a gamma amino butyric acid (GABA) reuptake inhibitor agent selected from nipecotic acid; a pro-drug of the GABA reuptake inhibitor agent, a precursor of the GABA reuptake inhibitor agent; and any combination thereof, wherein the neurodegenerative disease is treated. In various embodiments, -T is any one of the structures XXXIX-XLI, or pharmaceutically acceptable salt thereof:

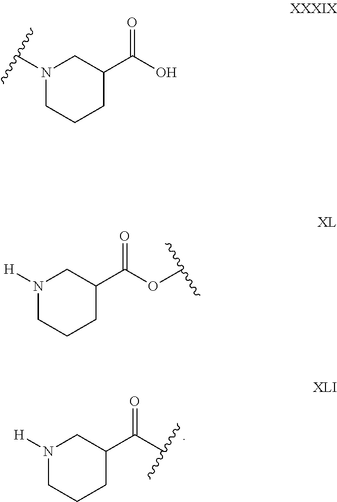 [SMILES: *N1CCCC(C(=O)O)C1 ; *OC(=O)C1CCCNC1 ; *C(=O)C1CCCNC1]

In some embodiments of the method for treating the neurodegenerative disease is administering a therapeutically effective amount of the huperzine or huperzine analog conjugate of formula I, wherein -T is a monoamine oxidase B (MAO-B) inhibitor agent selected from lazabemide, pargyline, rasagiline, selegiline, entacapone, tolcapone, nitecapone, and quercetin; a pro-drug of the MAO-B inhibitor agent, a precursor of the MAO-B inhibitor agent; and any combination thereof, wherein the neurodegenerative disease is treated. In various embodiments, T is one of structures XXII-LIb, or pharmaceutically acceptable salt thereof:

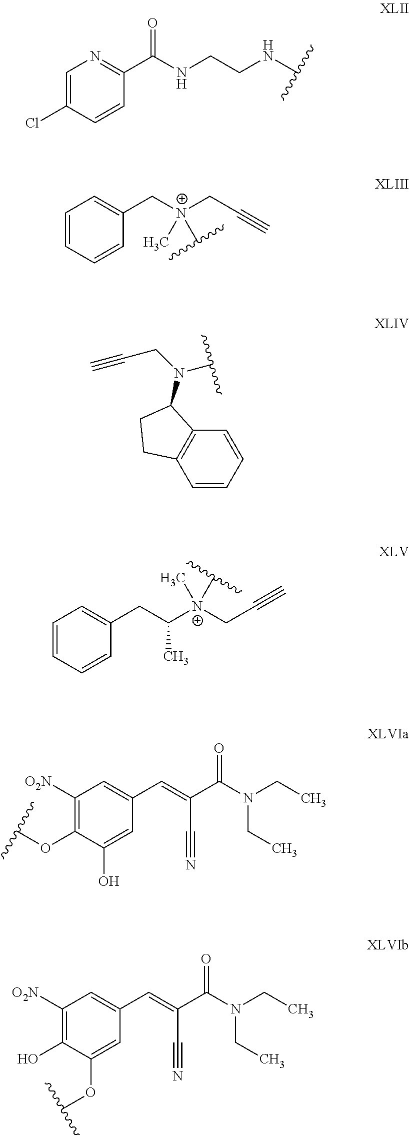

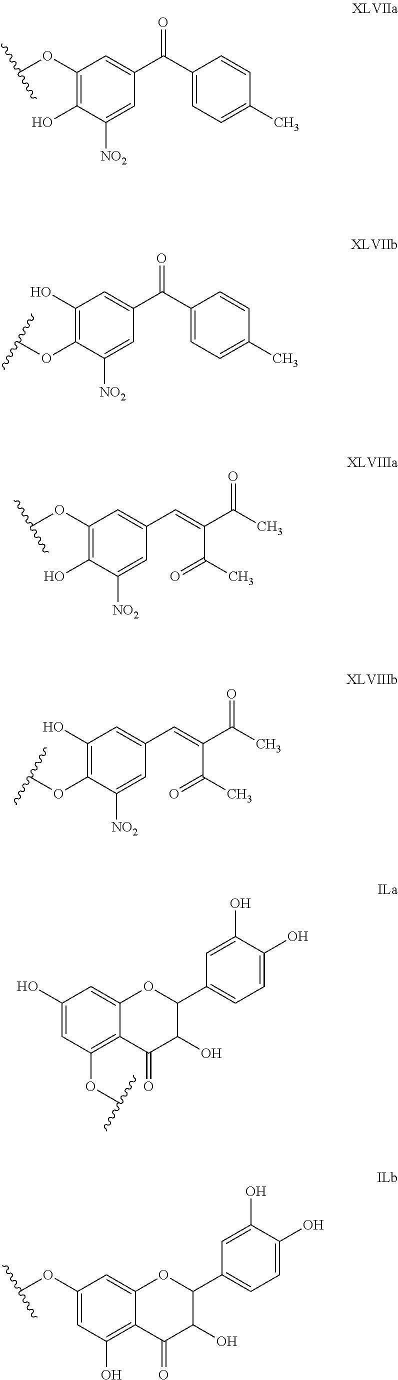 [SMILES: *Oc1cc(C(=O)c2ccc(C)cc2)cc([N+](=O)[O-])c1O ; *Oc1c(O)cc(C(=O)c2ccc(C)cc2)cc1[N+](=O)[O-] ; *Oc1cc(C=C(C(C)=O)C(C)=O)cc([N+](=O)[O-])c1O ; *Oc1c(O)cc(C=C(C(C)=O)C(C)=O)cc1[N+](=O)[O-] ; *Oc1cc(O)cc2c1C(=O)C(O)C(c1ccc(O)c(O)c1)O2 ; *Oc1cc(O)c2c(c1)OC(c1ccc(O)c(O)c1)C(O)C2=O]

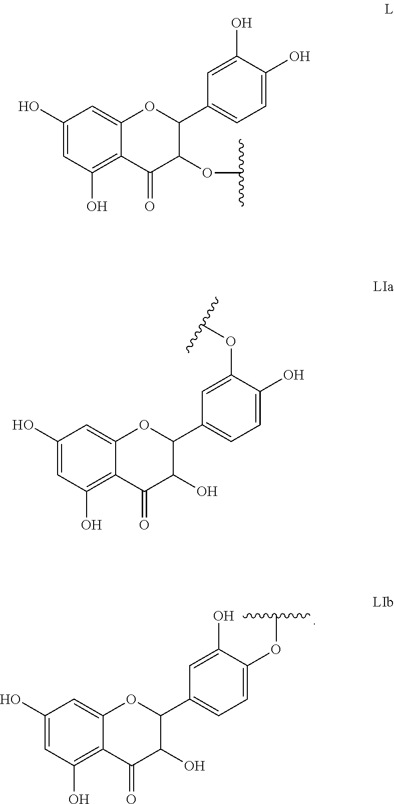 [SMILES: *OC1C(=O)c2c(O)cc(O)cc2OC1c1ccc(O)c(O)c1 ; *Oc1cc(C2Oc3cc(O)cc(O)c3C(=O)C2O)ccc1O ; *Oc1ccc(C2Oc3cc(O)cc(O)c3C(=O)C2O)cc1O]

In still other embodiments of the method for treating the neurodegenerative disease is administering a therapeutically effective amount of the huperzine or huperzine analog conjugate of formula I, wherein, -T is muscarinic receptor antagonist agent selected from atropine, cycycloverine, diphenhydramine, tolterodine, oxybutynin, opratropium, chlorpormazine, methoctramine, tripitramine, and gallamine; a pro-drug of the muscarinic receptor antagonist, a precursor of the muscarinic receptor antagonist; and any combination thereof, wherein the neurodegenerative disease is treated. In various embodiments, -T is any one of structures LII-LXIII, or pharmaceutically acceptable salt thereof:

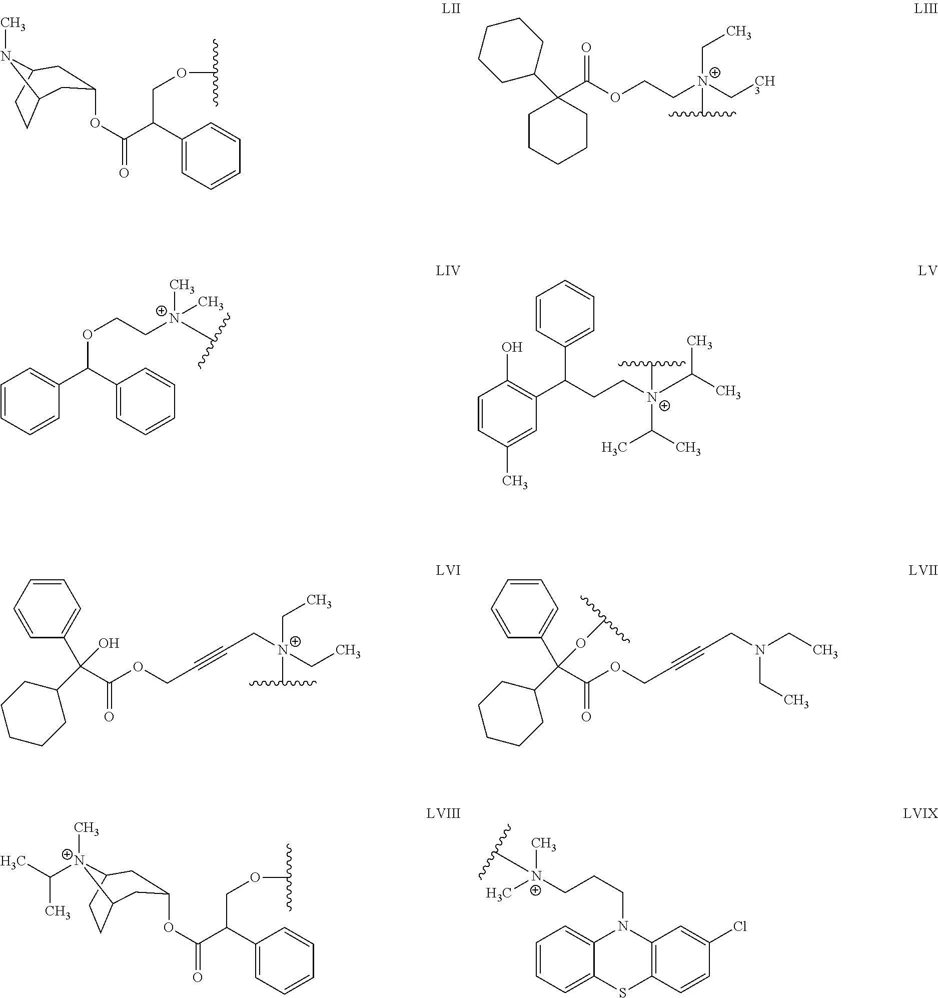

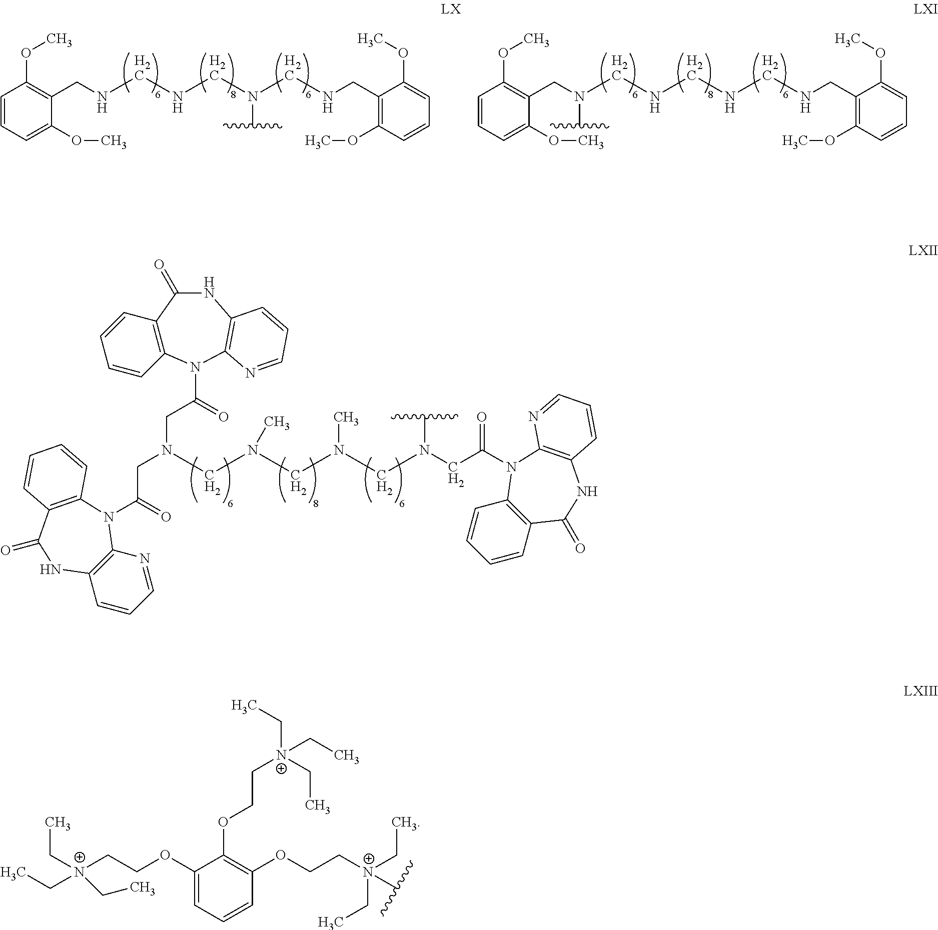

In some embodiments of the method for treating the neurodegenerative disease is administering a therapeutically effective amount of the huperzine or huperzine analog conjugate of formula I, wherein -T is a dopamine receptor antagonist agent selected from malperone, risperidone, ziprasidone, raclopride, clozapine, haloperidol, quetiapine, domperidone, eticlopride, yohimbine, blonanserin and L-741,626 (3-[4-(4-Chlorophenyl)-4-hydroxypiperidin-1-yl]methyl-1H-indole); a pro-drug of the dopamine receptor antagonist, a precursor of the dopamine receptor antagonist, and any combination thereof, wherein the neurodegenerative disease is treated. In various embodiments, -T is any one of structures LIX-LXXIII, or pharmaceutically acceptable salt thereof:

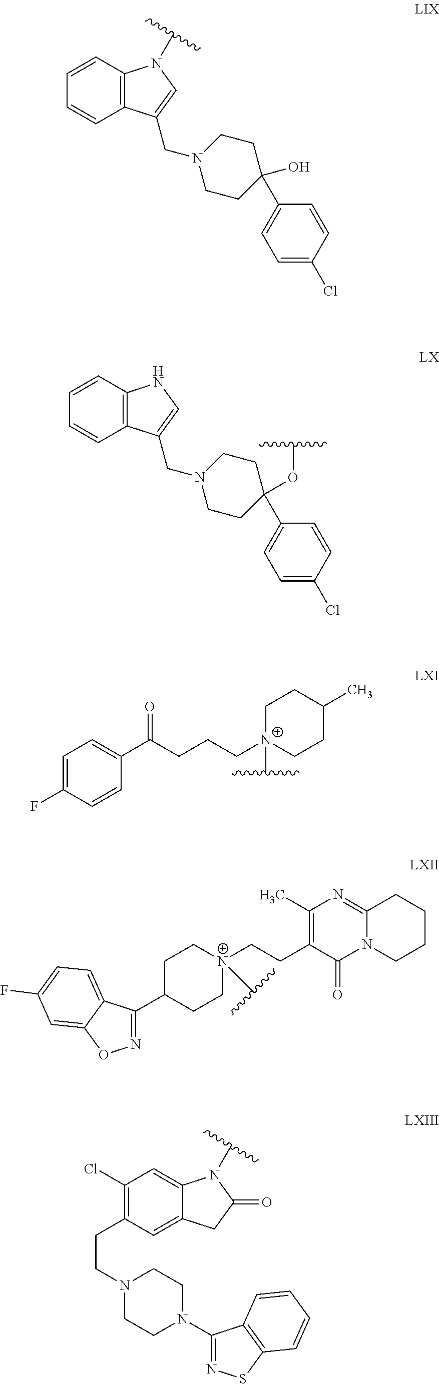 [SMILES: *n1cc(CN2CCC(O)(c3ccc(Cl)cc3)CC2)c2ccccc21 ; *OC1(c2ccc(Cl)cc2)CCN(Cc2c[nH]c3ccccc23)CC1 ; *[N+]1(CCCC(=O)c2ccc(F)cc2)CCC(C)CC1 ; *[N+]1(CCc2c(C)nc3n(c2=O)CCCC3)CCC(c2noc3cc(F)ccc23)CC1 ; *N1C(=O)Cc2cc(CCN3CCN(c4nsc5ccccc45)CC3)c(Cl)cc21]

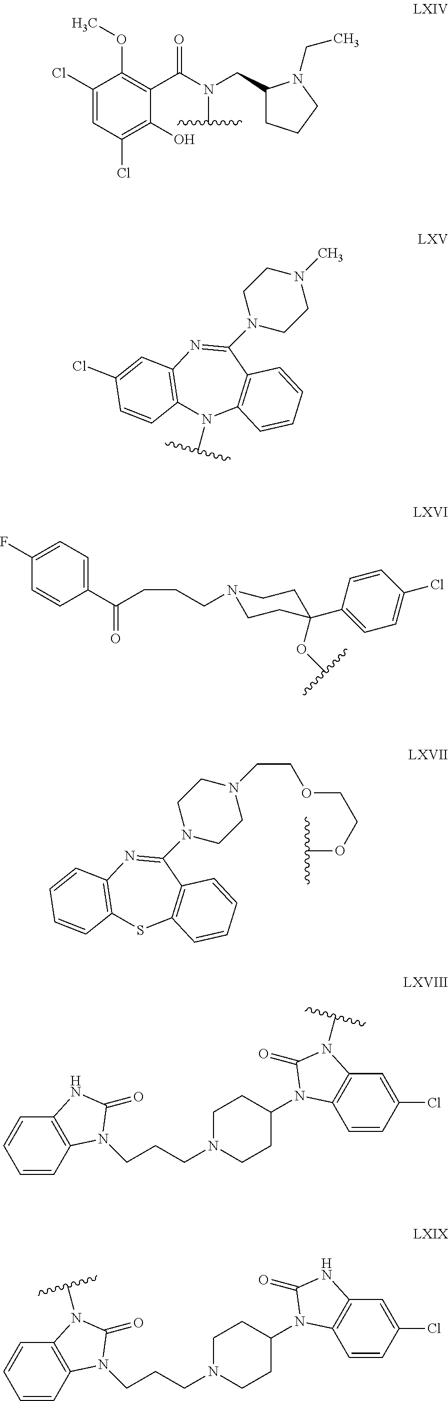

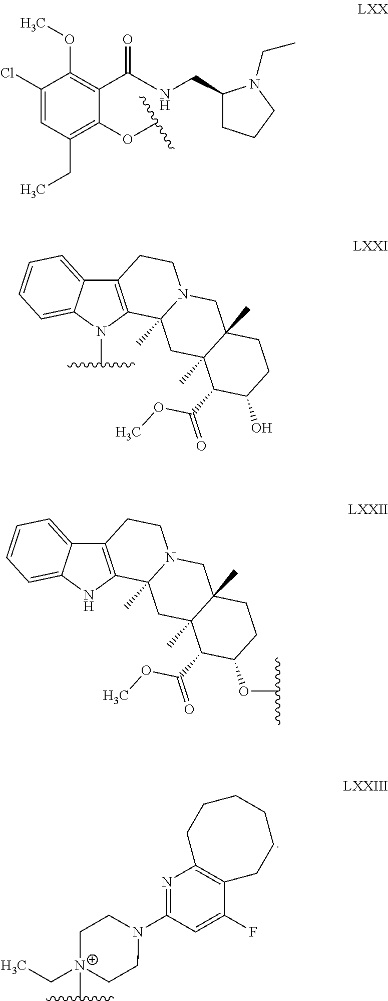

In some embodiments of the method for treating the neurodegenerative disease is administering a therapeutically effective amount of the huperzine or huperzine analog conjugate of formula I, wherein -T is a glutamate receptor (NR2B) antagonist agent selected from ifenprodil; a pro-drug of the NR2B antagonist, a precursor of the NR2B antagonist, and any combination thereof, wherein the neurodegenerative disease is treated. In various embodiments, -T is any one of structures LXXIV-LXXV, or pharmaceutically acceptable salt thereof:

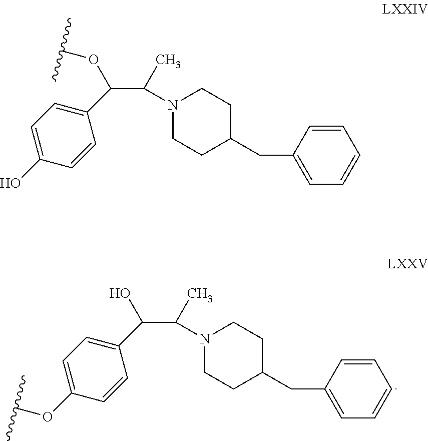 [SMILES: *OC(c1ccc(O)cc1)C(C)N1CCC(Cc2ccccc2)CC1 ; *Oc1ccc(C(O)C(C)N2CCC(Cc3ccccc3)CC2)cc1]

In some embodiments of the method for treating the neurodegenerative disease is administering a therapeutically effective amount of the huperzine or huperzine analog conjugate of formula I, wherein -T is epigallocatechin gallate (EGCG); a pro-drug of EGCG, a precursor of EGCG, and any combination thereof. In various embodiments, -T is any one of structures LXXVIa-LXVIIf, or pharmaceutically acceptable salt thereof

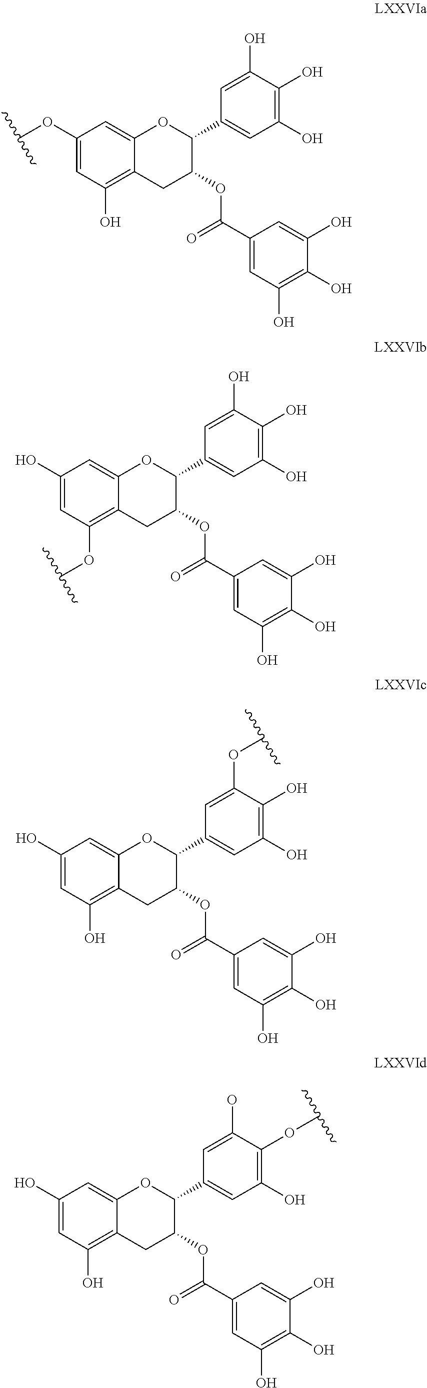 [SMILES: *Oc1cc(O)c2c(c1)O[C@H](c1cc(O)c(O)c(O)c1)[C@H](OC(=O)c1cc(O)c(O)c(O)c1)C2 ; *Oc1cc(O)cc2c1C[C@@H](OC(=O)c1cc(O)c(O)c(O)c1)[C@@H](c1cc(O)c(O)c(O)c1)O2 ; *Oc1cc([C@H]2Oc3cc(O)cc(O)c3C[C@H]2OC(=O)c2cc(O)c(O)c(O)c2)cc(O)c1O ; *Oc1c(O)cc([C@H]2Oc3cc(O)cc(O)c3C[C@H]2OC(=O)c2cc(O)c(O)c(O)c2)cc1O]

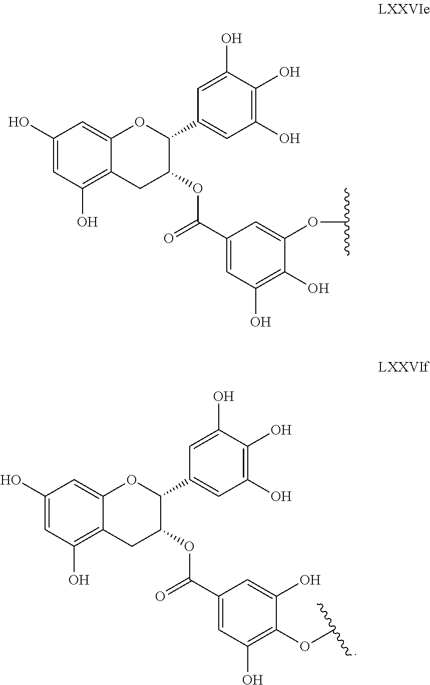 [SMILES: *Oc1cc(C(=O)O[C@@H]2Cc3c(O)cc(O)cc3O[C@@H]2c2cc(O)c(O)c(O)c2)cc(O)c1O ; *Oc1c(O)cc(C(=O)O[C@@H]2Cc3c(O)cc(O)cc3O[C@@H]2c2cc(O)c(O)c(O)c2)cc1O]

In still other embodiments of the method for treating the neurodegenerative disease is administering a therapeutically effective amount of the huperzine or huperzine analog conjugate of formula I, wherein -T is an aromatase inhibitor agent selected from aminoglutethimide and formestane; a prodrug of the aromatase inhibitor, a precursor of the aromatase inhibitor, and any combination thereof, wherein the neurodegenerative disease is treated. In various embodiments, -T is any one of structures LXVII-LXXVIII, or pharmaceutically acceptable salt thereof:

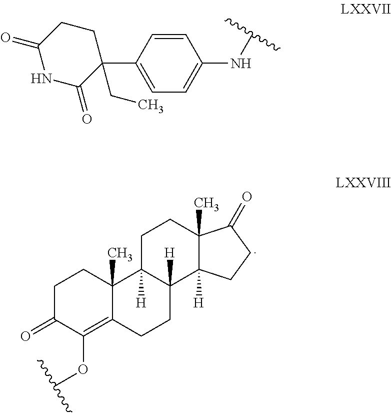

In still other embodiments of the method for treating the neurodegenerative disease is administering a therapeutically effective amount of the huperzine or huperzine analog conjugate of formula I, wherein -T is one of the following structures:

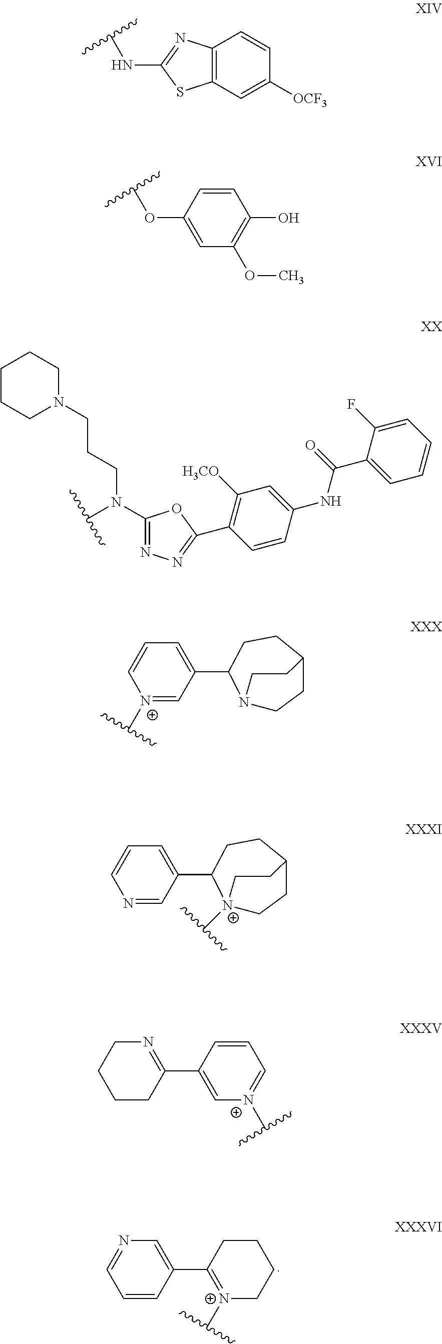

or pharmaceutically acceptable salt thereof, wherein the neurodegenerative disease is treated.

In still other embodiments of the method for treating the neurodegenerative disease is administering a therapeutically effective amount of the huperzine or huperzine analog conjugate of the following structure:

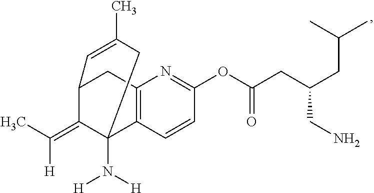 [SMILES: C/C=C1\C2C=C(C)CC1(N)c1ccc(OC(=O)C[C@@H](CN)CC(C)C)nc1C2]

or pharmaceutically acceptable salt thereof, wherein the neurodegenerative disease is treated.

In still other embodiments of the method for treating the neurodegenerative disease is administering a therapeutically effective, amount of the huperzine or huperzine analog conjugate of the following structure:

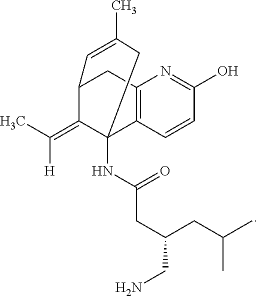 [SMILES: C/C=C1\C2C=C(C)CC1(NC(=O)C[C@@H](CN)CC(C)C)c1ccc(O)nc1C2]

or pharmaceutically acceptable salt thereof, wherein the neurodegenerative disease is treated.

In yet another embodiment of the method for treating the neurodegenerative disease is administering a therapeutically effective amount of the huperzine or huperzine analog conjugate of the following structure:

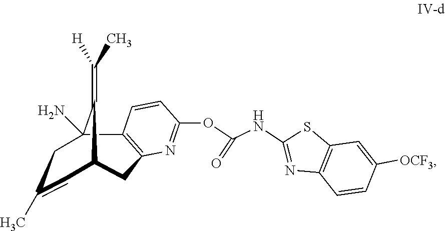

or pharmaceutically acceptable salt thereof, wherein the neurodegenerative disease is treated.

In still other embodiments of the method for treating the neurodegenerative disease is administering a therapeutically effective amount of the huperzine or huperzine analog conjugate of the following structure:

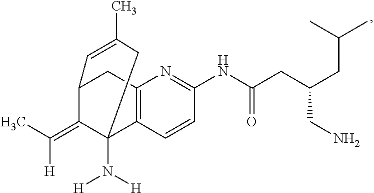 [SMILES: C/C=C1\C2C=C(C)CC1(N)c1ccc(NC(=O)C[C@@H](CN)CC(C)C)nc1C2]

or pharmaceutically acceptable salt thereof, wherein the neurodegenerative disease is treated.

In still another embodiment of the method for treating the neurodegenerative disease is administering a therapeutically effective amount of the huperzine or huperzine analog conjugate of the following structure:

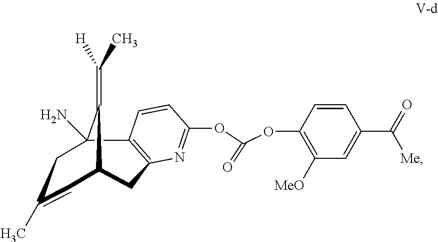

or pharmaceutically acceptable salt thereof, wherein the neurodegenerative disease is treated.

In still another embodiment of the method for mating the neurodegenerative disease is administering a therapeutically effective amount of the huperzine or huperzine analog conjugate of the following structure:

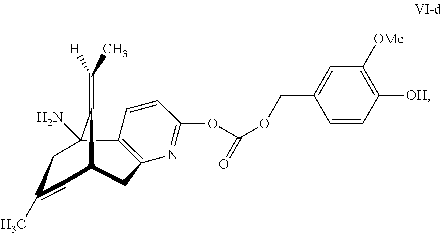

or pharmaceutically acceptable salt thereof, wherein the neurodegenerative disease is treated.

In still another embodiment of the method for treating the neurodegenerative disease is administering a therapeutically effective amount of the huperzine of huperzine analog conjugate of the following structure:

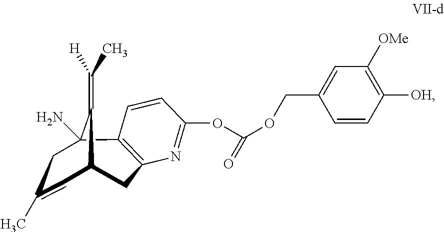

or pharmaceutically acceptable salt thereof, wherein the neurodegenerative disease is treated.

In still another embodiment of the method for treating the neurodegenerative disease is administering a therapeutically effective amount of the huperzine or huperzine analog conjugate of the following structure:

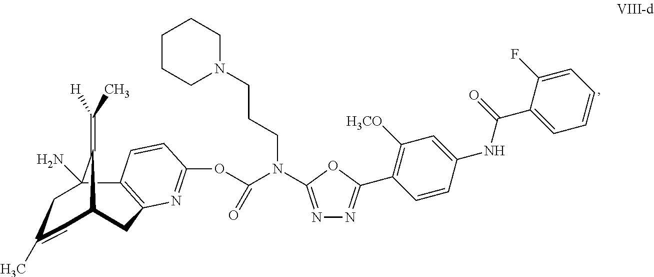

or pharmaceutically acceptable salt thereof, wherein the neurodegenerative disease is treated.

In still another embodiment of the method for treating the neurodegenerative disease is administering a therapeutically effective amount of the huperzine or huperzine analog conjugate of the following structure:

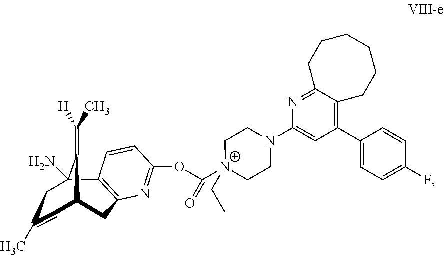

or pharmaceutically acceptable salt thereof, wherein the neurodegenerative disease is treated.

In still another embodiment of the method for treating the neurodegenerative disease is administering a therapeutically effective amount of the huperzine or huperzine analog conjugate of the following structure:

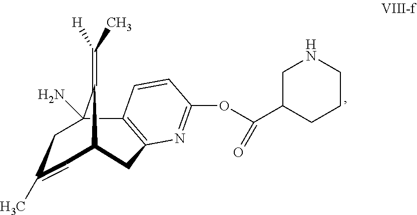

or pharmaceutically acceptable salt thereof, wherein the neurodegenerative disease is treated.

In still another embodiment of the method thr treating the neurodegenerative disease is administering a therapeutically effective amount of the huperzine or huperzine analog conjugate of the following structure:

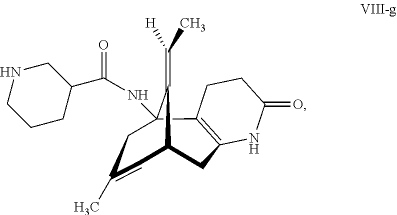

or pharmaceutically acceptable salt thereof, wherein the neurodegenerative disease is treated.

In still another embodiment of the method for treating the neurodegenerative disease is administering a therapeutically effective amount of the huperzine or huperzine analog conjugate of the following structure:

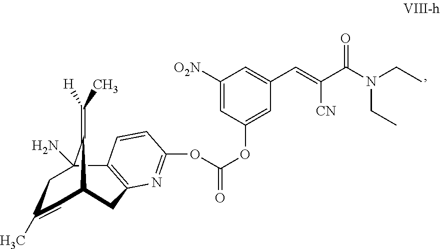

or pharmaceutically acceptable salt thereof, wherein the neurodegenerative disease is treated.

In still another embodiment of the method for treating the neurodegenerative disease is administering a therapeutically effective amount of the huperzine or huperzine analog conjugate of the following structure:

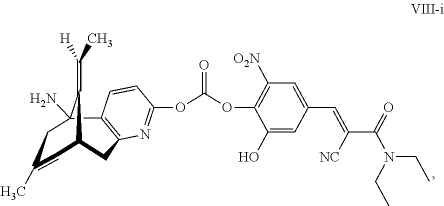

or pharmaceutically acceptable salt thereof, wherein the neurodegenerative disease is treated.

In some aspects, the present disclosure is directed to methods of delivering a therapeutic agent by administering a conjugate of a huperzine or huperzine analog, wherein the therapeutic agent is delivered to a target organ. In some embodiments, the target organ is the central nervous system. In other embodiments, the target organ is the brain.

An embodiment of the method for delivering a therapeutic agent by administering a therapeutically effective amount of a conjugate of huperzine or an analog of huperzine having the general formula:

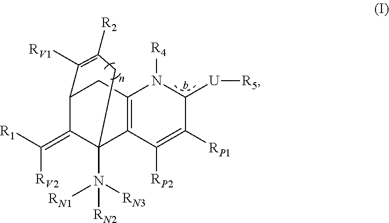

tautomer thereof, or pharmaceutically acceptable salt thereof, wherein R₁ is selected from H, (C₁-C₂₄)alkyl, CF₃, CF₂CF₃, CF₂CF₂CF₃, SO₂CH₃, SO₂Ph, SO₂Ar, SO₃H, and SO₃Ar, and —CH₂-L-T; R₂ is selected from H, (C₁-C₂₄)alkyl, aryl, cycloalkyl, (C₂-C₂₄)alkenyl, heterocycle, heteroaryl and —CH₂-L-T; R_{P1}, R_{P2}, R_{V1}, R_{V2} are independently selected from hydrogen and fluorine; R_{N1} is selected from H, (C₁-C₂₄)alkyl, CF₃, CF₂CF₃, CCl₃, CBr₃, CHO, and -L-T; R_{N1} and R_{N2} is selected from H, (C₁-C₂₄)alkyl, CF₃, CF₂CF₃, CCl₃, CBr₃, and CHO; R_{N3} is selected from absent and (C₁-C₂₄)alkyl; U is O, S, NH, or N((C₁-C₂₄)alkyl); b is a keto-enol tautomer unsaturation; and n is an integer selected from 1, 2, 3, and 4; R₄ is absent, or selected from H, and -L-T; R₅ is absent, or selected from H, and -L-T; at least one of R₁, R₂, R_{N1}, R₄ and R₅ is -L-T; only one of R_{N1}, R₄ and R₅ is -L-T; each is independently a linker that is selected from a bond, —O—, —S—, —NH—, —N(alkyl)-, —C(O)—, —O(C═O)—, —C(═O)—, —C(═S)O—, —C(═S)—, and —P(O)₂—; and each -T is independently selected from a therapeutic agent, a therapeutic agent pro-drug, or a therapeutic agent precursor, wherein the therapeutic agent is delivered to the target organ. In some embodiments, n=1, and R₁═R₂═CH₃.

In some embodiments of the method, the conjugate has R_{P1} is H or F; R_{P2} is H or F; R_{V1} is H or F; R_{V2} is H or F; wherein at least one of R_{P1}, R_{P2}, R_{V1}, and R_{V2} is fluorine. In another embodiment of the method, the conjugate has R_{P1} is H or F; is H or F; R_{V1} is H or F; R_{V2} is H or F; wherein at least one of R_{P1}, R_{P2}, R_{V1}, and R_{V2} is fluorine; R₁ is methyl; R₂ is methyl; R_{N1} is H; R_{N2} is H; and R_{N3} is absent.

In some embodiments of the method for delivering a therapeutic agent by administering a therapeutically effective amount of a conjugate wherein the linker may comprise at least one of a linker functional group selected from a bond. —O—, —S—, —NH—, —N(alkyl)-, —C(O)—, —O(C═O)—, —C(═O)O—, —C(═S)O—, —C(═S)—, and —P(O)₂—, wherein the therapeutic agent is delivered to the target organ. In various embodiments, the linker is a bond. In other embodiments, the linker is —C(O)—. In other embodiments, the linker is —C(═S)—. In still other embodiments, the linker is —P(O)₂—. In yet other embodiments, the linker is an ether, sulfide, or an amine. In some embodiments, the linker is —O(C═O)—, or in other embodiments, —C(═O)O—.

In some embodiments of the method, the conjugate administered has each -T therapeutic agent, therapeutic agent pro-drug, or therapeutic agent precursor is independently —V—W—X—Y—Z, wherein V is bond, —O—, or —NH—; W is —(C₀-C₆)alkyl-, —(C₂-C₆)alkenyl-; or —(C₂-C₆)alkynyl-; X is a bond, —O—, —NH—, —CO—, —(C═O)NH—, —NH—(C═O)—, —(C═NH)—NH—, —(C═O)—O—, or —O(C═O)—; Y is a —(C₀-C₆)alkyl-, —(C₂-C₆)alkenyl-; or —(C₂-C₆)alkynyl-; Z is a -quaternary amine, -cycloalkyl, -aryl, -heterocycle, or heteroaryl; and each nitrogen, alkyl, alkenyl, alkynyl, eyeloalkyl, aryl, heterocycle, or heteroaryl is optionally substituted, wherein the therapeutic agent is delivered to the target organ.

In still other embodiments of the method for delivering a therapeutic agent is administering a therapeutically effective amount of the huperzine or huperzine analog conjugate of formula I, wherein -T is a covalently bonded glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor; a precursor to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor, a gamma amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof, Wherein the therapeutic agent is delivered to the target organ.

In still other embodiments of the method for delivering a therapeutic agent is administering a therapeutically effective amount of the huperzine or huperzine analog conjugate of formula I, wherein -T is —(C═O)—CH₂—CH(i-Bu)-CH₂—NH₂, wherein the therapeutic agent is delivered to the target organ. In other embodiments, -T is (S)—(C═O)—CH₂—CH(i-Bu)-CH₂—NH₂.

In yet other embodiments of the method for delivering a therapeutic agent is administering a therapeutically effective amount of the huperzine or huperzine analog conjugate of formula I, wherein -T is a covalently bonded N-methyl d-aspartate receptor antagonist selected from R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantadine, dextrallorphan, dextromethorphan, dextrorphan, dizocilpine, eticyclidine, gacyclidine, ibogaine memantine, methoxetamine, phencyclidine, rolicyclidine, tenocyclidine, methoxydine, tiletamine, neramexane, eliprodil, etoxadrol, dexoxadrol, remacemide, delucemine, 8a-phenyidecahydroquinoline, aptiganel, rhynchophylline, ketamine, 1-aminocyclopropanecarboxylic acid, 7-chlorokynurenate, 5,7-dichlorokynurenic acid, kynurenic acid, lacosamide; a pro-drug of the NMDA receptor antagonist, a precursor of the NMDA receptor antagonist; and any combination thereof.

In still other embodiments of the method for delivering a therapeutic agent is administering a therapeutically effective amount of the huperzine or huperzine analog conjugate of formula I, wherein the compound is one wherein -T is any one of the structures XLXXIX-CXVIII, or pharmaceutically acceptable salt thereof:

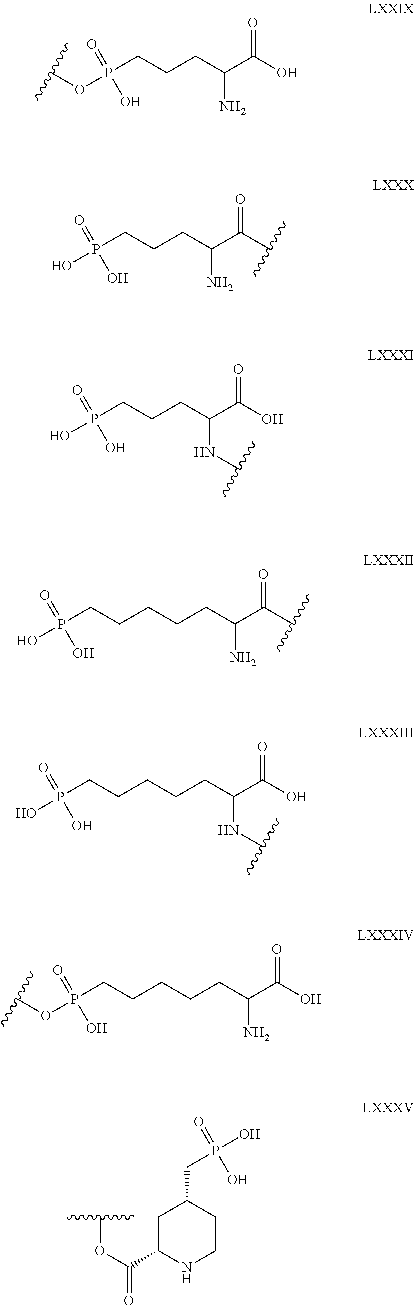

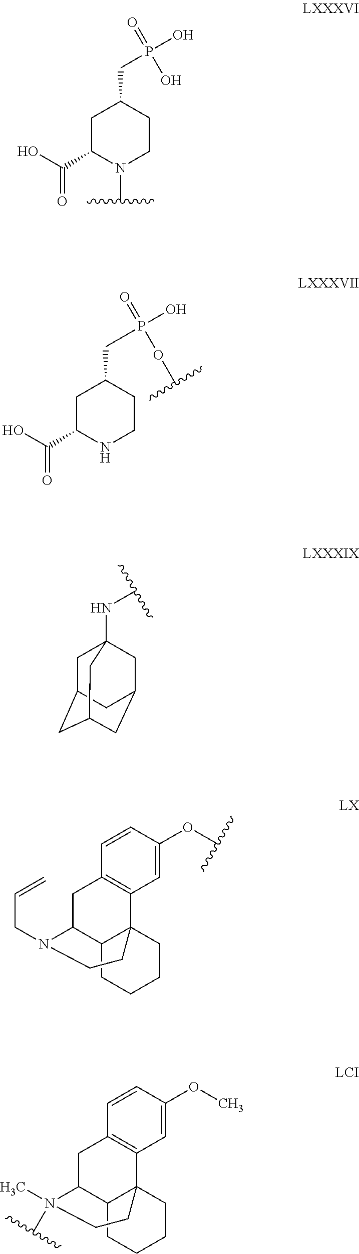

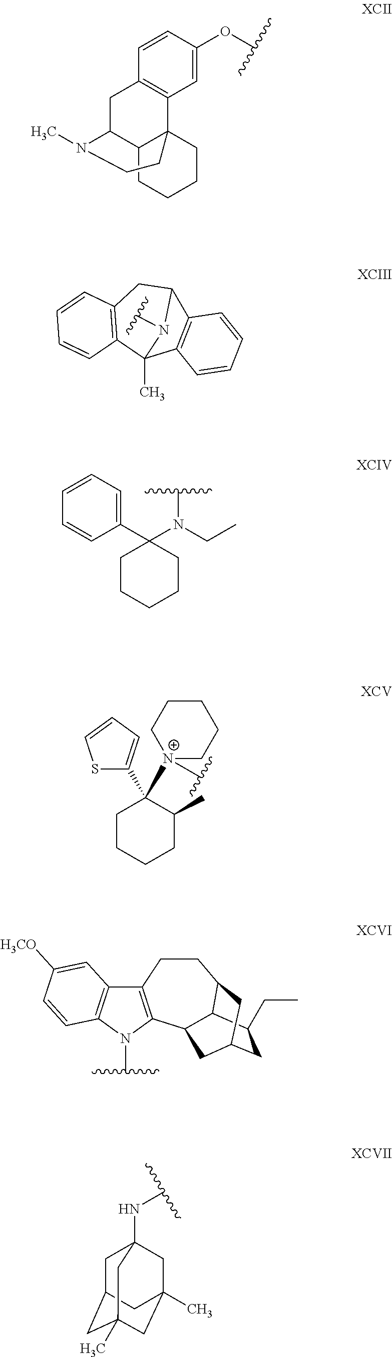 [SMILES: *Oc1ccc2c(c1)C13CCCCC1C(C2)N(C)CC3 ; *N1C2Cc3ccccc3C1(C)c1ccccc12 ; *N(CC)C1(c2ccccc2)CCCCC1 ; *[N+]1([C@]2(c3cccs3)CCCC[C@@H]2C)CCCCC1 ; *n1c2c(c3cc(OC)ccc31)CC[C@H]1CC3C[C@H](CC)C1[C@H]2C3 ; *NC12CC3CC(C)(CC(C)(C3)C1)C2]

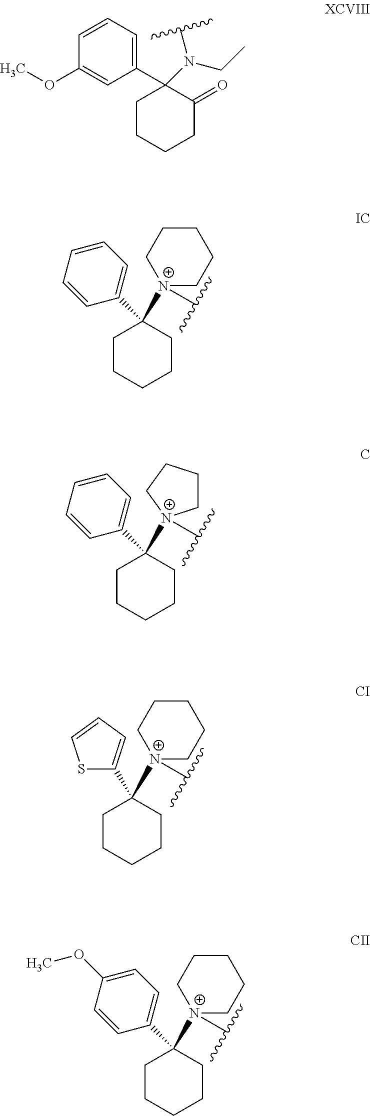

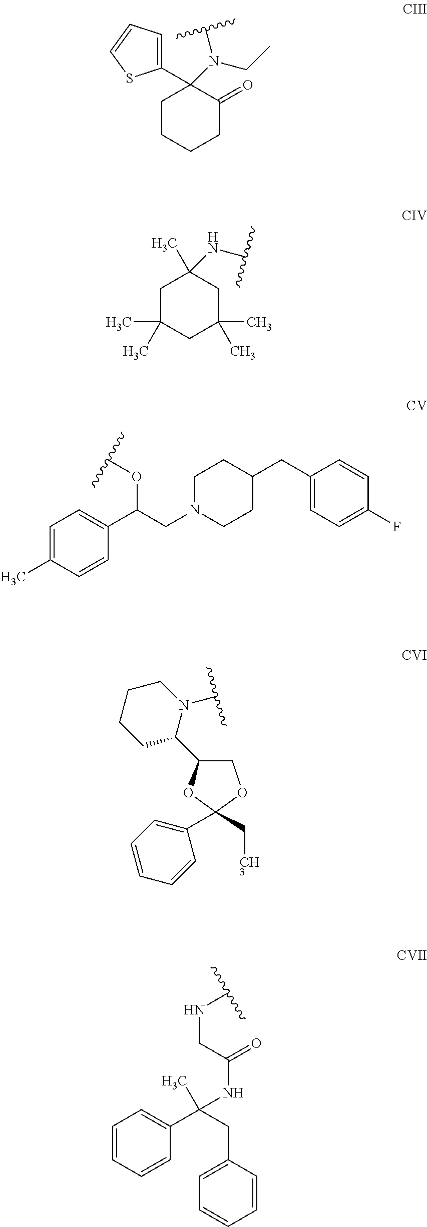

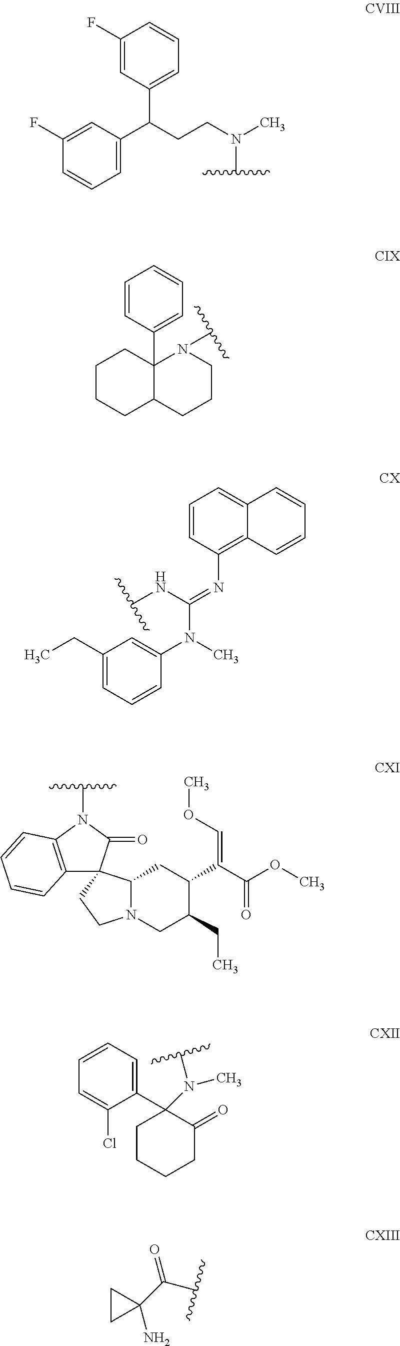

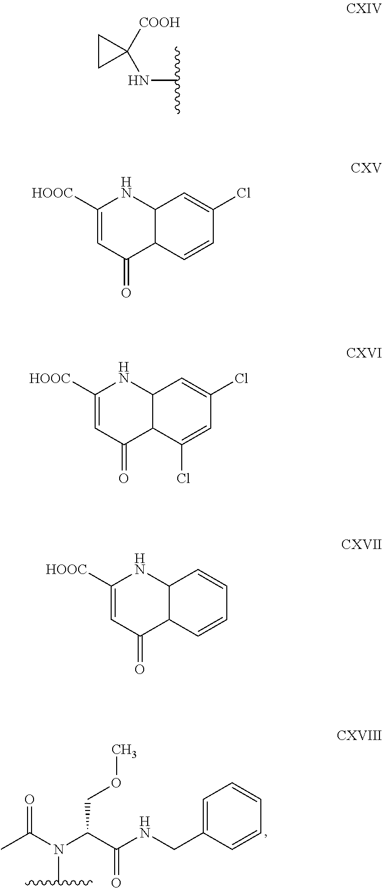 [SMILES: *NC1(C(=O)O)CC1 ; O=C(O)C1=CC(=O)C2C=CC(Cl)=CC2N1 ; O=C(O)C1=CC(=O)C2C(Cl)=CC(Cl)=CC2N1 ; O=C(O)C1=CC(=O)C2C=CC=CC2N1 ; *N(C(C)=O)[C@H](COC)C(=O)NCc1ccccc1]

wherein the therapeutic agent is delivered to the target organ.

In some embodiments of the method for delivering a therapeutic agent is administering a therapeutically effective amount of the huperzine or huperzine analog conjugate of formula I, wherein the compound is one in which -T is a covalently bonded mitochondrial protectant selected from bethanechol and (2S,2′R,3′S,5′R)-1-methyl-2-(2-methyl-1,3-oxathiolan-5-yl)pyrrol-idine 3-sulfoxide methyl iodide; a pro-drug of the mitochondrial protectant, a precursor of the mitochondrial protectant; and any combination thereof, wherein the therapeutic agent is delivered to the target organ. In various embodiments, -T is any one of the structures CXIX-CXX, or pharmaceutically acceptable salt thereof:

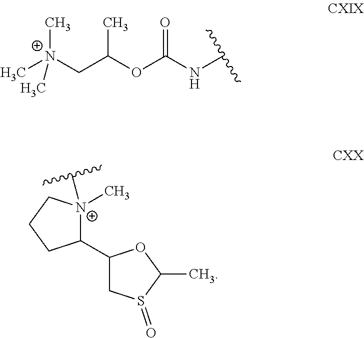 [SMILES: *NC(=O)OC(C)C[N+](C)(C)C ; *[N+]1(C)CCCC1C1CS(=O)C(C)O1]

In other embodiments of the method for delivering a therapeutic agent is administering a therapeutically effective amount of the huperzine or huperzine analog conjugate of formula I, wherein the compound is one in which -T is a covalently bonded anti-inflammatory agent selected from non-steroidal anti-inflammatory drugs, immune-selective anti-inflammatory derivatives, and anti-inflammatory herbal extracts, NF-Kappa B inhibitors, and IL-inhibitors; a pro-drug of the anti-inflammatory agent, a precursor of the anti-inflammatory agent; and any combination thereof, wherein the therapeutic agent is delivered to the target organ.

In still other embodiments of the method, the compound is one where -T is a covalently bonded antioxidant; a pro-drug to an antioxidant; or a precursor to an antioxidant. Examples of anti-oxidants which may be conjugated with huperzine or an analog thereof include, but are not limited to, ascorbic acid, glutathione, lipoic acid, uric acid, beta-carotene, vitamin A, vitamin E, co-enzyme Q; a pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (EGC), epicatechin gallate (ECG), and epigallocatechin inflate (EGCG), epigallocatechin and gallic acid, methyl gallate, and any combination thereof.

In yet other embodiments of the method, the compound is one wherein -T is the following structure or pharmaceutically acceptable salt thereof bonded through any one of NH, NH₂, COOH, OH, thiol, or an enol-tautomer oxygen of one of the following compounds: uric acid, ascorbic acid, glutathione, melatonin, alpha-tocopherol, beta-tocopherol, gamma-tocopherol, delta-tocopherol, alpha-tocotrienols beta-tocotrienol, gamma-tocotrienol, delta, tocotrienol, catechin epicatechin, epigallocatechin, epicatechin gallate, and epigallocatechin gallate, epigallocatechin, gallic acid, methyl gallate, or a combination thereof.

In still other embodiments of the method, the compound is one where T is a covalently bonded anti-inflammatory agent; a pro-drug to an anti-inflammatory agent; or a precursor to an anti-inflammatory agent. Examples of anti-inflammatory agents which may be conjugated with huperzine or an analog thereof include, but are not limited to, compounds of non-steroidal anti-inflammatory drugs, immune-selective anti-inflammatory derivatives, anti-inflammatory herbal extracts, extracts of salix purpurea, extracts of piper longum, extracts of boswellia serrata and extracts of prunella vulgaris, NF-Kappa B inhibitors, IL-inhibitors, any combinations thereof, and/or the like. Additional examples of anti-inflammatory agents include, but are not limited to, tanshinone, eryptotanshinone, ferulic acid, cycloartenyl, cycloartenyl ferulate, hydroxy tyrosol, homovanillyl alcohol, 4-O-methylgallic acid, and any combination thereof.

In yet other embodiments of the method, the compound is one wherein -T is the following structure or pharmaceutically acceptable salt thereof bonded through any one of COOH or OH of one of the following compounds: ferulic acid, cycloartenyl, cycloartenyl ferulate, hydroxytyrosol, homovanillyl alcohol, 4-O-methylgallic acid, and any combination thereof.

In some embodiments of the method for delivering a therapeutic agent is administering a therapeutically effective amount of the huperzine or huperzine analog conjugate of formula I, wherein -T is a covalently bonded alpha-7 agonist selected from 1,3,4-oxadiazol-2-amine, (+)—N-(1-azabicyclo[2.2.2]oct-3-yl)benzo[b]furan-2-carboxamide, AR-R17779, TC-5619, GTS-21, PHA-543,613, PNU-282,987, SSR-180,711, tropisetron, WAY-317,538, choline, and nicotine (37-38); a pro-drug of the alpha-7 agonist; a precursor of the alpha-7 agonist; and any combination thereof, wherein the therapeutic agent is delivered to the target organ. In various embodiments, -T is any one of the structures XXI-XXV, XXV, XXVII-XXVIII, XXXII-XXXIV, XXXVII-XXXVIII, or pharmaceutically acceptable salt thereof:

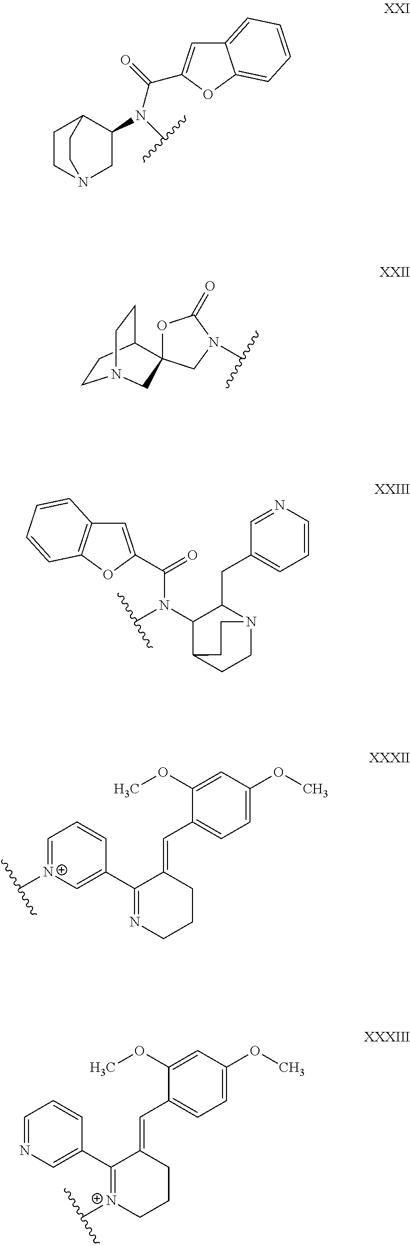 [SMILES: *N(C(=O)c1cc2ccccc2o1)[C@H]1CN2CCC1CC2 ; *N1C[C@@]2(CN3CCC2CC3)OC1=O ; *N(C(=O)c1cc2ccccc2o1)C1C2CCN(CC2)C1Cc1cccnc1 ; *[n+]1cccc(C2=NCCC/C2=C\c2ccc(OC)cc2OC)c1 ; *[N+]1=C(c2cccnc2)/C(=C/c2ccc(OC)cc2OC)CCC1]

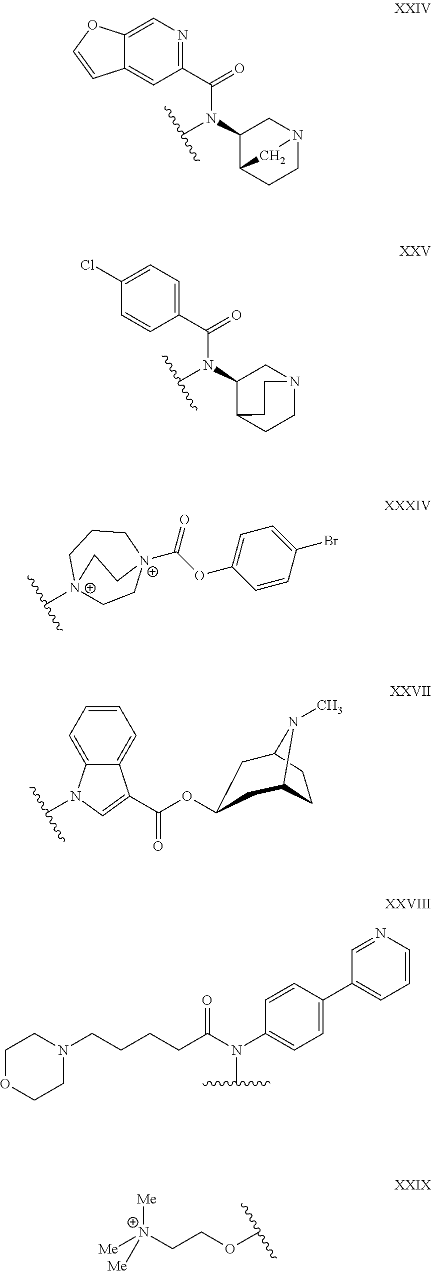

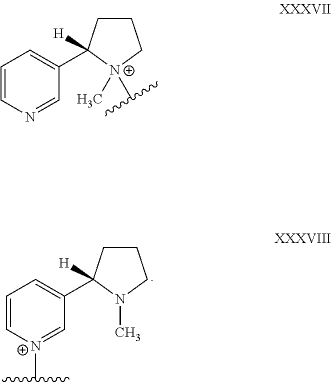 [SMILES: *[N+]1(C)CCC[C@H]1c1cccnc1 ; *[n+]1cccc([C@@H]2CCCN2C)c1]

In some embodiments of the method for delivering a therapeutic agent is administering a therapeutically effective amount of the huperzine or huperzine analog conjugate of formula I, wherein the compound is one in which T is a covalently hound potassium channel blocker selected from dofetilide, sotalol, ibutilide, azimilide, nifekalant, tedisamil, sematilide, 4-aminopyridine, and 3,4-diaminopyridine; a pro-drug of the potassium channel blocker; a precursor of the potassium channel blocker; and any combination thereof, wherein the therapeutic agent is delivered to the target organ. In various embodiments, -T is any one of the structures XVII-XIX, or pharmaceutically acceptable salt thereof:

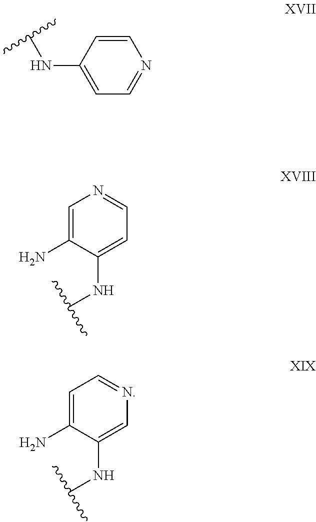 [SMILES: *Nc1ccncc1 ; *Nc1ccncc1N ; *Nc1cnccc1N]

In still other embodiments of the method for delivering a therapeutic agent is administering a therapeutically effective amount of the huperzine or huperzine analog conjugate of formula I, wherein -T is a sodium channel blocker selected from propranolol, procainamide, quinidine, disopyramide, lidocane, mexiletine, tocainide, phenytoin, encainide, flecainide, moricizine, propafenone, riluzole; a pro-drug of the sodium channel Mocker; a precursor of the sodium channel blocker; and any combination thereof, wherein the therapeutic agent is delivered to the target organ. In yet other embodiments, -T is an anticonvulsant selected from pregabalin, (S)-pregabalin, gabapentin, stiripentol, phenobarbital, methylphenobarbital, barbexaclone, lorazepam, nitrazepam, temazepam, nimetazepam, felbamate, carbamazepine, oxcarbazepine, eslicarbazepine acetate, valproic acid, vigabatrin, progabide, tiagabine, topiramate, ethotoin, phenytoin, mephenytoin, fosphenytoin, pheneturide, beclamide, primidone, brivaracetam, levetiracetam, seletracetam, ethosuximide, acetazolamide, sultiame, phenacemide, methazolamide, zonisamide, lamotrigine; a pro-drug of the anticonvulsant, a precursor of the anticonvulsant; and any combination thereof.

In still other embodiments of the method for delivering a therapeutic agent is administering a therapeutically effective amount: of the huperzine or huperzine analog conjugate of formula I, wherein -T is an anxiolytic agent selected from positive allosteric modulators of GABA receptor, serotonin-specific re-uptake inhibitors (SSRI), barbiturates, and benzodiazepines; a pro-drug of the anxiolytic agent, a precursor of the anxiolytic agent; and any combination thereof, wherein the therapeutic agent is delivered to the target organ. In yet other embodiments, -T is an anxiolytic agent selected from carisoprodol, glutethimide, meprobamate, propofol, theanine, hydroxyzine, valerenic acid, niacin, niacinamide; a pro-drug of the anxiolytic agent, a precursor of the anxiolytic agent; and any combination thereof.

In some embodiments of the method for delivering a therapeutic agent is administering a therapeutically effective amount of the huperzine or huperzine analog conjugate of formula I, wherein -T is an NADPH oxidase inhibitor agent selected from apocynin, a pro-drug of the NADPH oxidase inhibitor agent, a precursor of the NADPH oxidase inhibitor agent; and any combination thereof, wherein the therapeutic agent is delivered to the target organ. In one embodiment, -T has the structure structures XV, or pharmaceutically acceptable salt thereof:

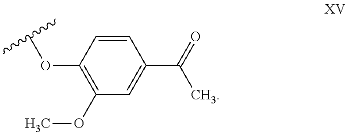 [SMILES: *Oc1ccc(C(C)=O)cc1OC]

In still other embodiments of the method for delivering a therapeutic agent is administering a therapeutically effective amount of the huperzine or huperzine analog conjugate of formula I, wherein -T is a gamma amino butyric acid (GABA) reuptake inhibitor agent selected from nipecotic acid; a pro-drug of the GABA reuptake inhibitor agent, a precursor of the GABA reuptake inhibitor agent and any combination thereof, wherein the therapeutic agent is delivered to the target organ. In various embodiments, -T is any one of the structures XXXIX-XLI, or pharmaceutically acceptable salt thereof:

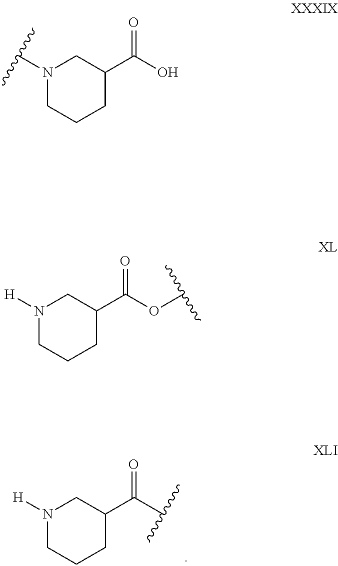 [SMILES: *N1CCCC(C(=O)O)C1 ; *OC(=O)C1CCCNC1 ; *C(=O)C1CCCNC1]

In some embodiments of the method for delivering a therapeutic agent is administering a therapeutically effective amount of the huperzine or huperzine analog conjugate of formula t, wherein -T is a monoamine oxidase B (MAO-B) inhibitor agent selected from lazabemide, pargyline, rasagiline, selegiline, entacapone, tolcapone, nitecapone, and quercetin: a pro-drug of the MAO-B inhibitor agent, a precursor of the MAO-B inhibitor agent; and any combination thereof, wherein the therapeutic agent is delivered to the target organ. In various embodiments, T is one of structures XLII-LIb, or pharmaceutically acceptable salt thereof:

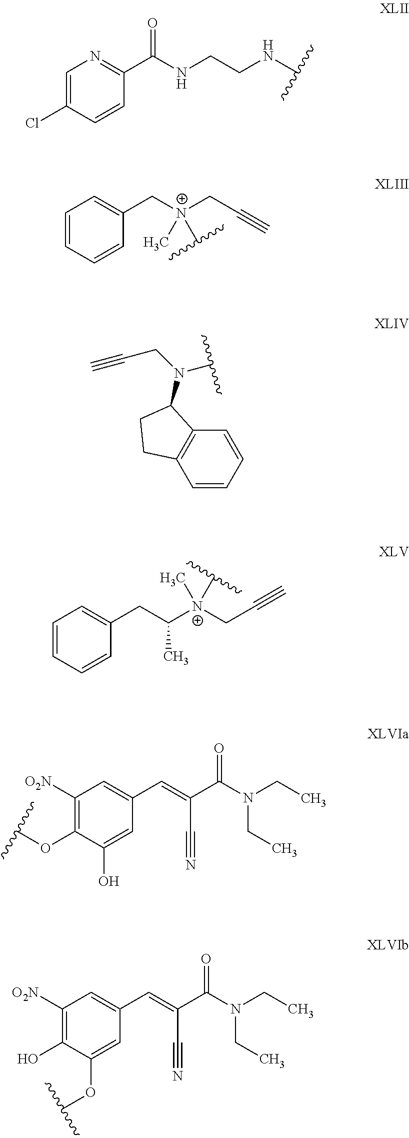

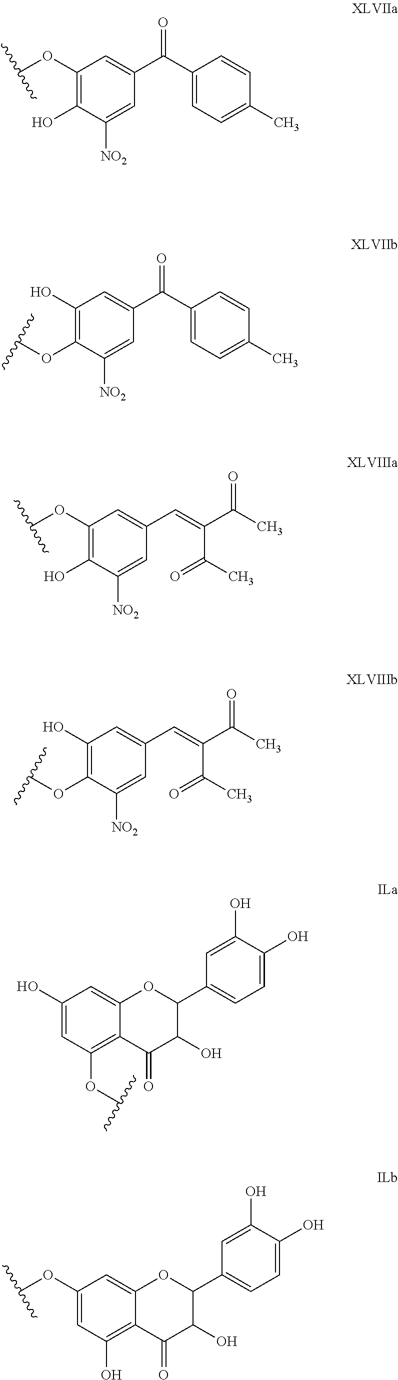 [SMILES: *Oc1cc(C(=O)c2ccc(C)cc2)cc([N+](=O)[O-])c1O ; *Oc1c(O)cc(C(=O)c2ccc(C)cc2)cc1[N+](=O)[O-] ; *Oc1cc(C=C(C(C)=O)C(C)=O)cc([N+](=O)[O-])c1O ; *Oc1c(O)cc(C=C(C(C)=O)C(C)=O)cc1[N+](=O)[O-] ; *Oc1cc(O)cc2c1C(=O)C(O)C(c1ccc(O)c(O)c1)O2 ; *Oc1cc(O)c2c(c1)OC(c1ccc(O)c(O)c1)C(O)C2=O]

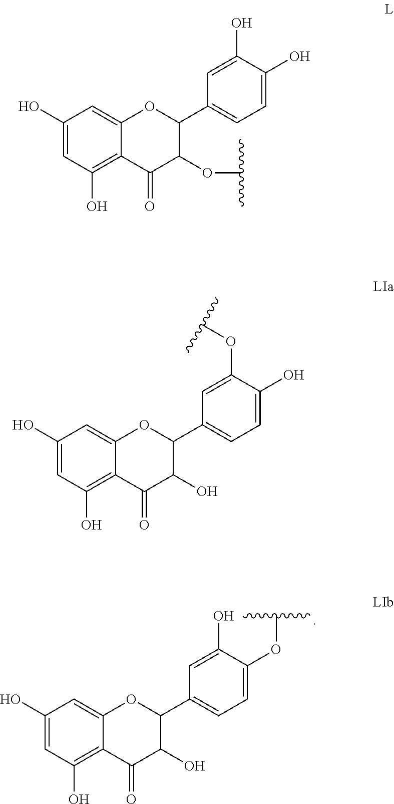 [SMILES: *OC1C(=O)c2c(O)cc(O)cc2OC1c1ccc(O)c(O)c1 ; *Oc1cc(C2Oc3cc(O)cc(O)c3C(=O)C2O)ccc1O ; *Oc1ccc(C2Oc3cc(O)cc(O)c3C(=O)C2O)cc1O]

In still other embodiments of the method for delivering a therapeutic agent is administering a therapeutically effective amount of the huperzine or huperzine analog conjugate of formula wherein, -T is muscarinic receptor antagonist agent selected from atropine, cycycloverine, diphenhydramine, tolterodine, oxybutynin, opratropium, chlorpormazine, methoctramine, tripitramine, and gallamine; a pro-drug of the muscarinic receptor antagonist, a precursor of the muscarinic receptor antagonist; and any combination thereof, wherein the therapeutic agent is delivered to the target organ. In various embodiments, -T is any one of structures LII-LXIII, or pharmaceutically acceptable salt thereof:

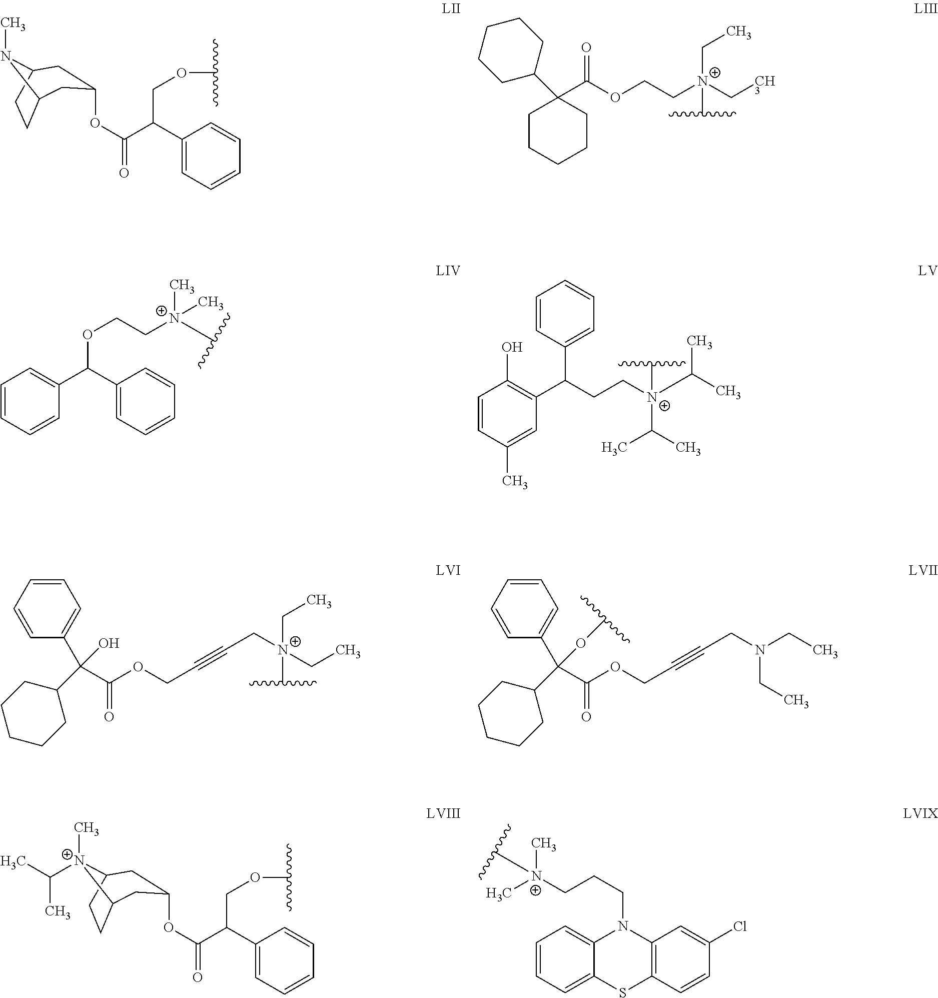

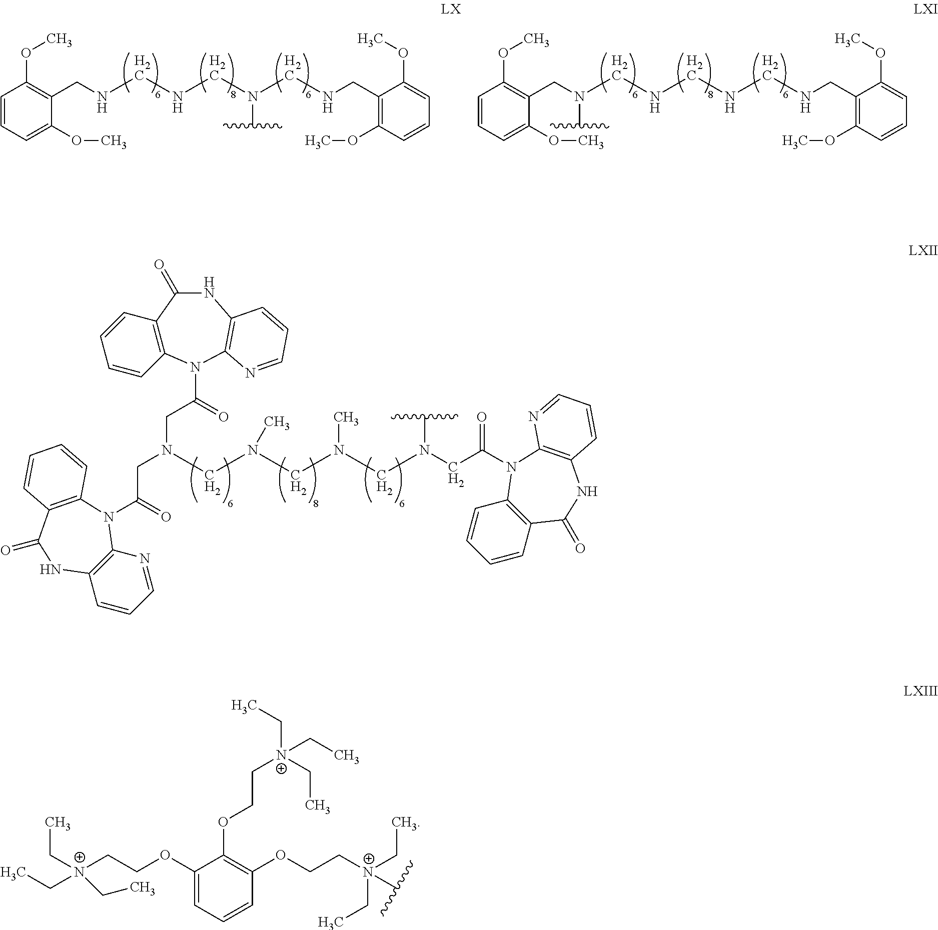

In some embodiments of the method for delivering a therapeutic agent is administering a therapeutically effective amount of the huperzine or huperzine analog conjugate of formula I, wherein is a dopamine receptor antagonist agent selected from malperone, risperidone, ziprasidone, raclopride, clozapine, haloperidol, quetiapine, domperidone, eticlopride, yohimbine, blonanserin and L-741,626 (3-[4-(4-Chlorophenyl)-4-hydroxypiperidin-1-yl]methyl-1H-indole); a pro-drug of the dopamine receptor antagonist, a precursor of the dopamine receptor antagonist, and any combination thereof, wherein the therapeutic agent is delivered to the target organ. In various embodiments, -T is any one of structures LIX-LXXIII, or pharmaceutically acceptable salt thereof:

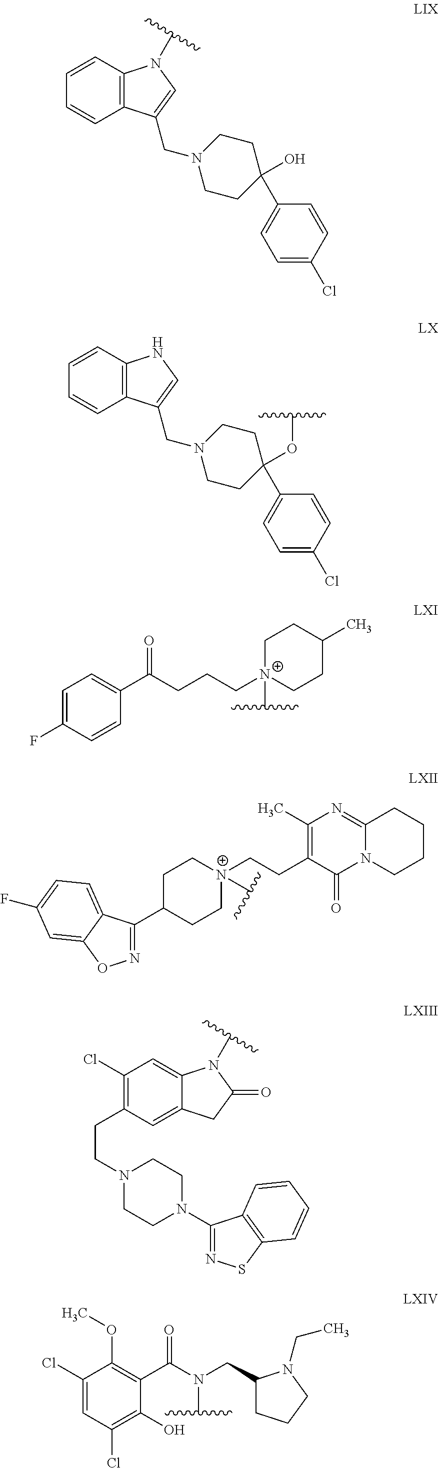

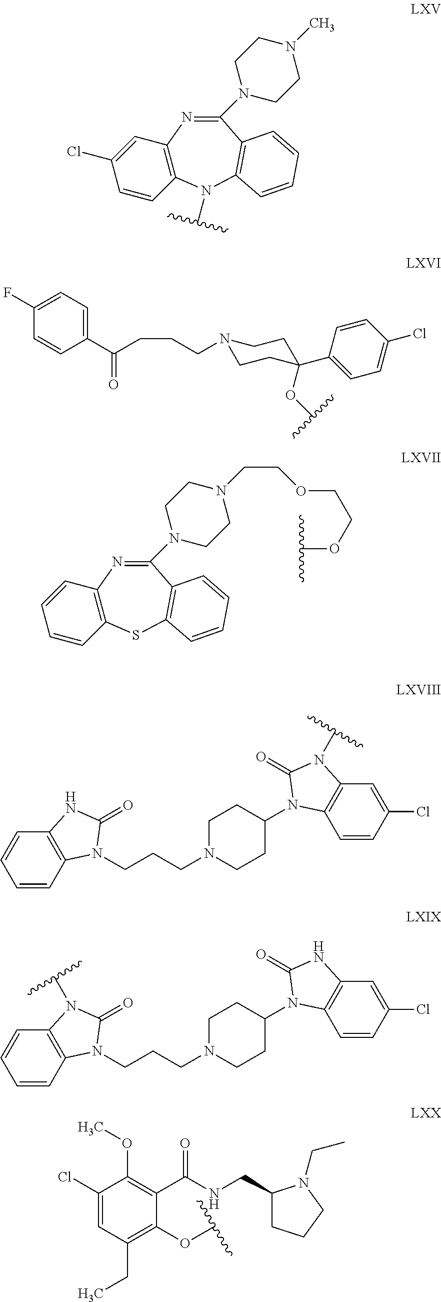 [SMILES: *N1c2ccc(Cl)cc2N=C(N2CCN(C)CC2)c2ccccc21 ; *OC1(c2ccc(Cl)cc2)CCN(CCCC(=O)c2ccc(F)cc2)CC1 ; *OCCOCCN1CCN(C2=Nc3ccccc3Sc3ccccc32)CC1 ; *n1c(=O)n(C2CCN(CCCn3c(=O)[nH]c4ccccc43)CC2)c2ccc(Cl)cc21 ; *n1c(=O)n(CCCN2CCC(n3c(=O)[nH]c4cc(Cl)ccc43)CC2)c2ccccc21 ; *Oc1c(CC)cc(Cl)c(OC)c1C(=O)NC[C@@H]1CCCN1CC]

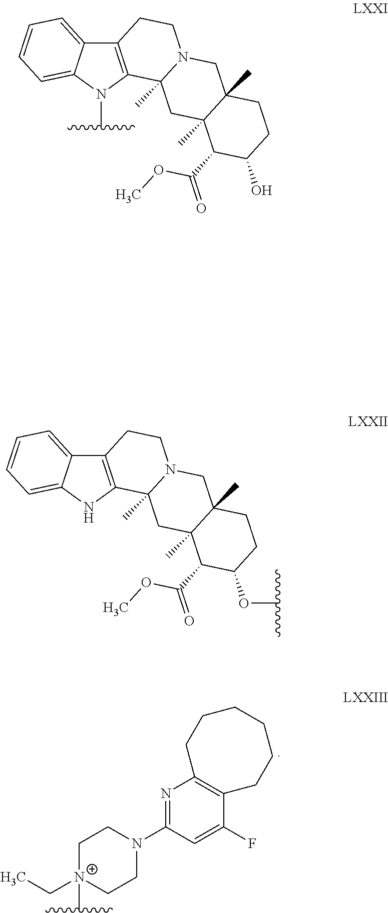 [SMILES: *n1c2c(c3ccccc31)CCN1C[C@]3(C)CC[C@H](O)[C@H](C(=O)OC)[C@@]3(C)C[C@@]21C ; *O[C@H]1CC[C@@]2(C)CN3CCc4c([nH]c5ccccc45)[C@]3(C)C[C@]2(C)[C@H]1C(=O)OC ; *[N+]1(CC)CCN(c2cc(F)c3c(n2)CCCCCC3)CC1]

In some embodiments of the method fir delivering a therapeutic agent is administering a therapeutically effective amount of the huperzine or huperzine analog conjugate of formula I, wherein -T is a glutamate receptor (NR2B) antagonist agent selected from ifenprodil; a pro-drug of the NR2B antagonist, a precursor of the NR2B antagonist, and any combination thereof, wherein the therapeutic agent is delivered to the target organ. In various embodiments, -T is any one of structures LXXIV-LXXV, or pharmaceutically acceptable salt thereof:

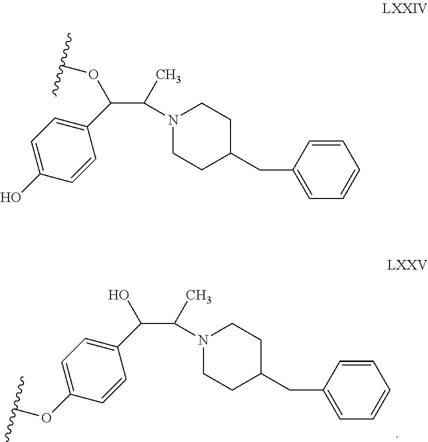 [SMILES: *OC(c1ccc(O)cc1)C(C)N1CCC(Cc2ccccc2)CC1 ; *Oc1ccc(C(O)C(C)N2CCC(Cc3ccccc3)CC2)cc1]

In some embodiments of the method for delivering a therapeutic agent is administering a therapeutically effective amount of the huperzine or huperzine analog conjugate of formula I, wherein -T is epigallocatechin gallate (EGCG); a pro-drug of EGCG, a precursor of EGCG, and any combination thereof. In various embodiments, -T is any one of structures LXXVIa-LXVIIf, or pharmaceutically acceptable salt thereof:

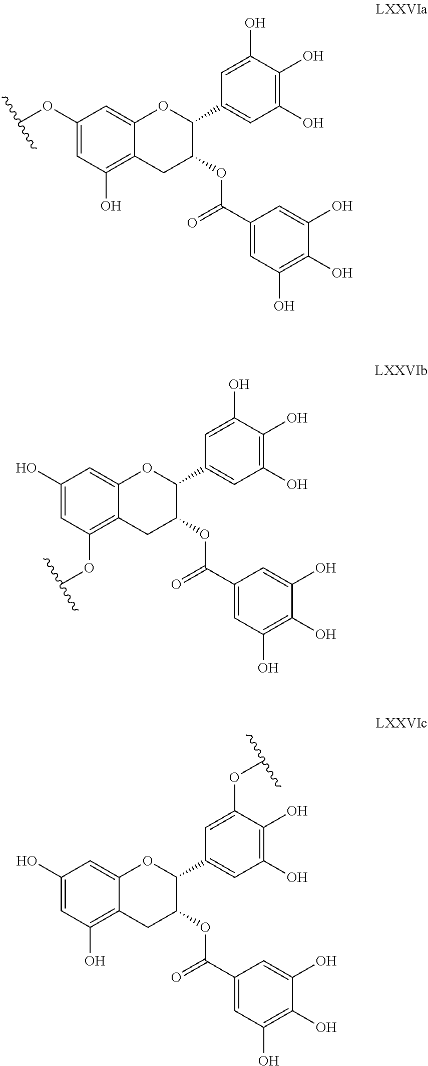 [SMILES: *Oc1cc(O)c2c(c1)O[C@H](c1cc(O)c(O)c(O)c1)[C@H](OC(=O)c1cc(O)c(O)c(O)c1)C2 ; *Oc1cc(O)cc2c1C[C@@H](OC(=O)c1cc(O)c(O)c(O)c1)[C@@H](c1cc(O)c(O)c(O)c1)O2 ; *Oc1cc([C@H]2Oc3cc(O)cc(O)c3C[C@H]2OC(=O)c2cc(O)c(O)c(O)c2)cc(O)c1O]

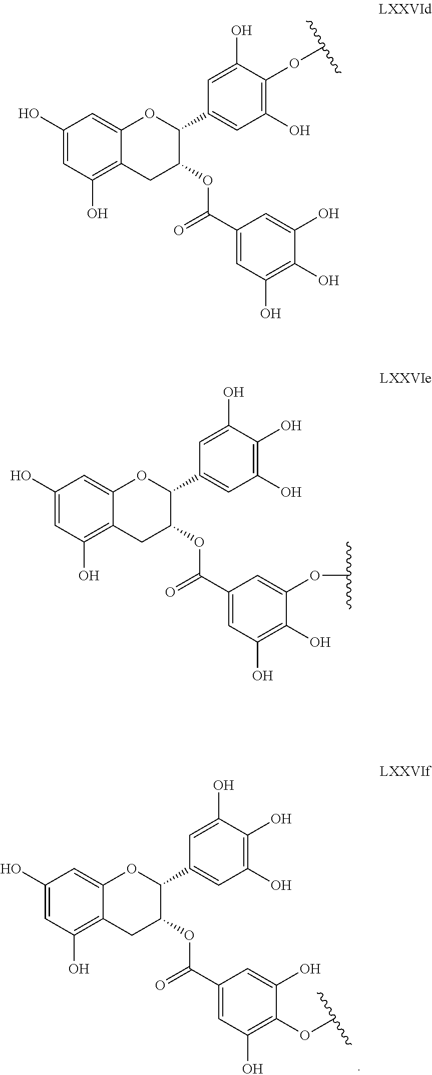 [SMILES: *Oc1c(O)cc([C@H]2Oc3cc(O)cc(O)c3C[C@H]2OC(=O)c2cc(O)c(O)c(O)c2)cc1O ; *Oc1cc(C(=O)O[C@@H]2Cc3c(O)cc(O)cc3O[C@@H]2c2cc(O)c(O)c(O)c2)cc(O)c1O ; *Oc1c(O)cc(C(=O)O[C@@H]2Cc3c(O)cc(O)cc3O[C@@H]2c2cc(O)c(O)c(O)c2)cc1O]

In still other embodiments of the method for delivering a therapeutic agent is administering a therapeutically effective amount of the huperzine or huperzine analog conjugate of formula I, wherein -T is an aromatase inhibitor agent selected from aminoglutethimide and formestane; a prodrug of the aromatase inhibitor, a precursor of the aromatase inhibitor, and any combination thereof, wherein the therapeutic agent is delivered to the target organ. In various embodiments, -T is any one of structures LXVII-LXXVIII, or pharmaceutically acceptable salt thereof:

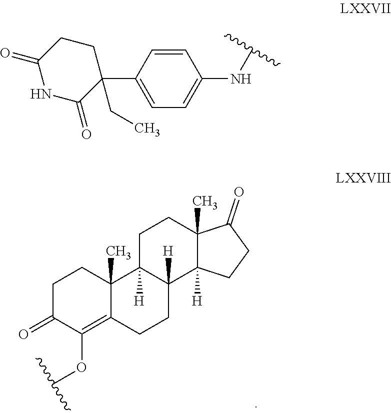

In still other embodiments of the method for delivering a therapeutic agent is administering a therapeutically effective amount of the huperzine or huperzine analog conjugate of formula I, wherein -T is one of the following structures:

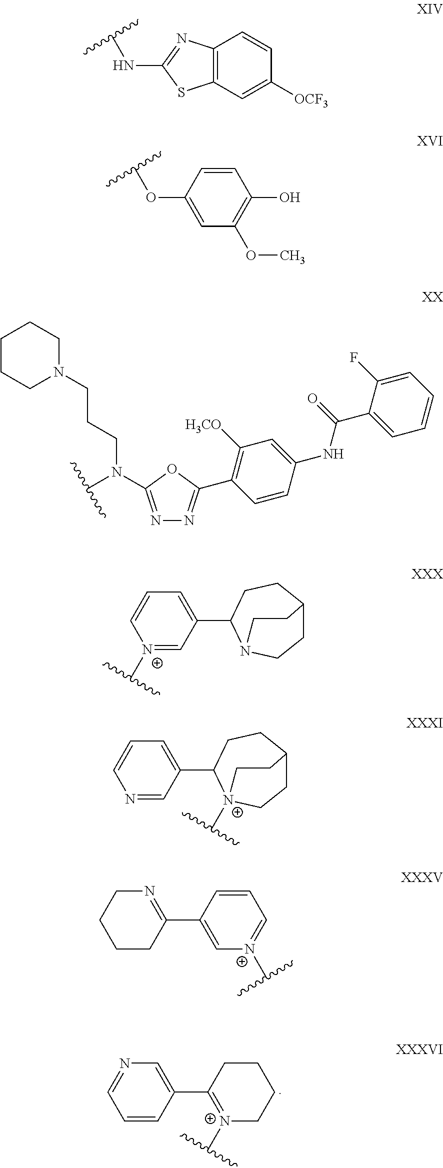

or pharmaceutically acceptable salt thereof, wherein the therapeutic agent is delivered to the target organ.

In still other embodiments of the method for delivering a therapeutic agent is administering a therapeutically effective amount of the huperzine or huperzine analog conjugate of the following structure:

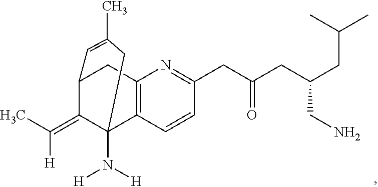 [SMILES: C/C=C1\C2C=C(C)CC1(N)c1ccc(CC(=O)C[C@@H](CN)CC(C)C)nc1C2]

or pharmaceutically acceptable salt thereof, wherein the therapeutic agent is delivered to the target organ.

In still other embodiments of the method for delivering a therapeutic agent is administering a therapeutically effective amount of the huperzine or huperzine analog conjugate of the following structure:

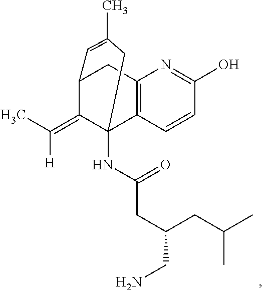 [SMILES: C/C=C1\C2C=C(C)CC1(NC(=O)C[C@@H](CN)CC(C)C)c1ccc(O)nc1C2]

or pharmaceutically acceptable salt thereof, wherein the therapeutic agent is delivered to the target organ.

In yet another embodiment of the method for delivering a therapeutic agent is administering a therapeutically effective amount of the huperzine or huperzine analog conjugate of the following structure:

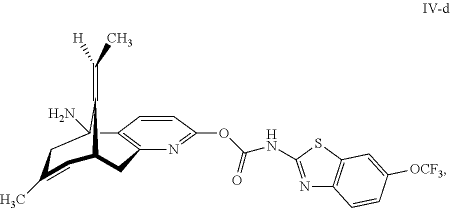

or pharmaceutically acceptable salt thereof, wherein the therapeutic agent is delivered to the target organ.

PHA in still other embodiments of the method for delivering a therapeutic agent is administering a therapeutically effective amount of the huperzine or huperzine analog conjugate of the following structure:

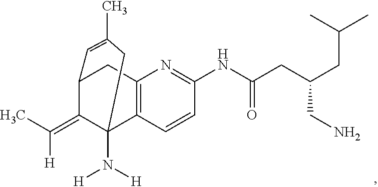 [SMILES: C/C=C1\C2C=C(C)CC1(N)c1ccc(NC(=O)C[C@@H](CN)CC(C)C)nc1C2]

or pharmaceutically acceptable salt thereof, wherein the therapeutic agent is delivered to the target organ.

In still another embodiment of the method for delivering a therapeutic agent is administering a therapeutically effective amount of the huperzine or huperzine analog conjugate of the following structure:

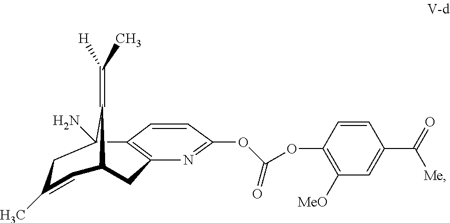

or pharmaceutically acceptable salt thereof, Wherein the therapeutic agent is delivered to the target organ.

In still another embodiment of the method for delivering a therapeutic agent is administering a therapeutically effective amount of the huperzine or huperzine analog conjugate of the following structure:

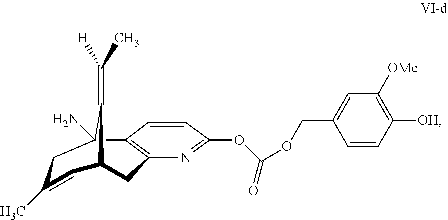

or pharmaceutically acceptable salt thereof, wherein the therapeutic went is delivered to the target organ.

In still another embodiment of the method for delivering a therapeutic agent is administering a therapeutically effective amount of the huperzine or huperzine analog conjugate of the following structure:

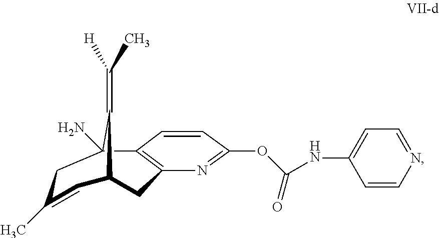

or pharmaceutically acceptable salt thereof, wherein the therapeutic agent: is delivered to the target organ.

In still another embodiment of the method for delivering a therapeutic agent is administering a therapeutically effective amount of the huperzine or huperzine analog conjugate of the following structure:

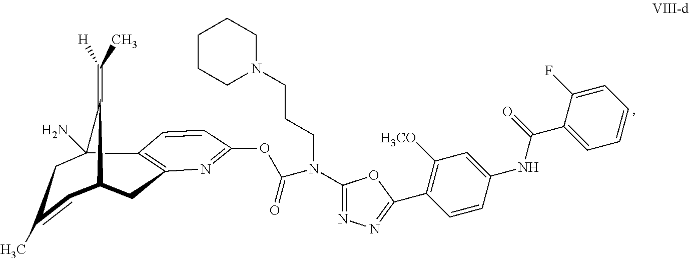

or pharmaceutically acceptable salt thereof, wherein the therapeutic agent is delivered to the target organ.

In still another embodiment of the method for delivering a therapeutic agent is administering a therapeutically effective amount of the huperzine or huperzine analog conjugate of the following structure:

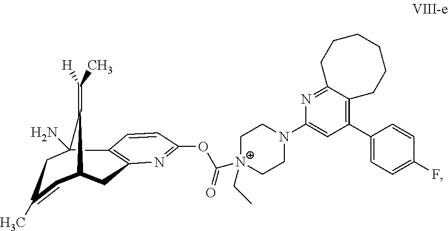

or pharmaceutically acceptable salt thereof, wherein the therapeutic agent is delivered to the target organ.

In still another embodiment of the method for delivering a therapeutic agent is administering a therapeutically effective amount of the huperzine or huperzine analog conjugate of the following structure:

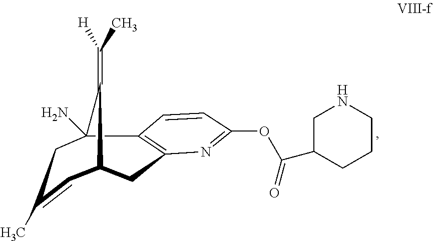

or pharmaceutically acceptable salt thereof, wherein the therapeutic agent is delivered to the target organ.

In still another embodiment of the method for delivering a therapeutic agent is administering a therapeutically effective amount of the huperzine or huperzine analog conjugate of the following structure:

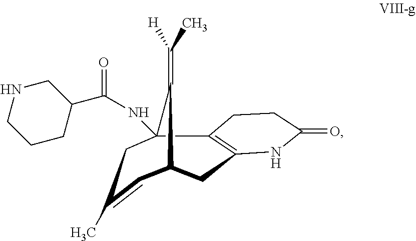

or pharmaceutically acceptable salt thereof, wherein the therapeutic agent is delivered to the target organ.

In still another embodiment of the method for delivering a therapeutic agent is administering a therapeutically effective amount of the huperzine or huperzine analog conjugate of the following structure:

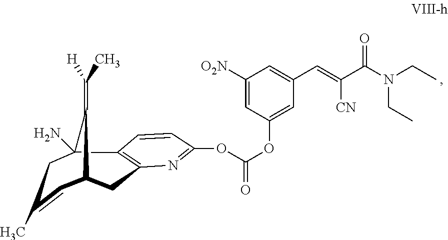

or pharmaceutically acceptable salt thereof, wherein the therapeutic anent is delivered to the target organ.

In still another embodiment of the method for delivering a therapeutic agent is administering a therapeutically effective amount of the huperzine or huperzine analog conjugate of the following structure:

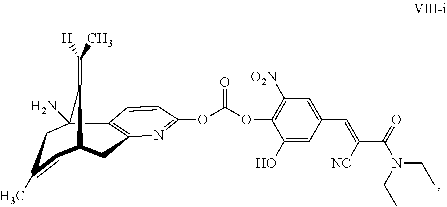

or pharmaceutically acceptable salt thereof, Wherein the therapeutic agent is delivered to the target organ.

In some aspects, the present disclosure is directed to methods of increasing the concentration of a therapeutic agent in a target organ by administering a conjugate of a huperzine or huperzine analog, wherein the concentration of the therapeutic agent is enhanced in a target organ. In some embodiments, the target organ is the central nervous system. In other embodiments, the target man is the brain.

An embodiment of the method of increasing the concentration of a therapeutic agent in a target organ by administering a therapeutically effective amount of a conjugate of huperzine or an analog of huperzine having the general formula:

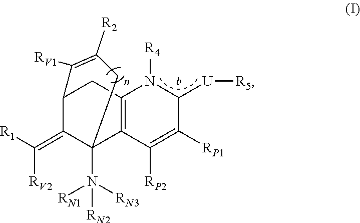

tautomer thereof, or pharmaceutically acceptable salt thereof, wherein R1 is selected from (C₁-C₂₄)alkyl, CF₃, CF₂CF₃, CF₂CF₂CF₃, SO₂CH₃, SO₂Ph, SO₂Ar, SO₃H, and SO₃Ar, and —CH₂-L-T; R₂ is selected from H, (C₁-C₂₄)alkyl, aryl, cycloalkyl, (C₂-C₂₄)alkenyl, heterocycle, heteroaryl and —CH₂-L-T; R_{P1}, R_{P2}, R_{V1}, R_{V2} are independently selected from hydrogen and fluorine; R_{N1} is selected from H, (C₁-C₂₄)alkyl, CF₃, CF₂CF₃, CCl₃, CBr₃, CHO, and -L-T; R_{N1} and R_{N2} is selected from H, (C₁-C₂₄)alkyl, CF₃, CF₂CF₃, CCl₃, CBr₃, and CHO; R_{N3} is selected from absent and (C₁-C₂₄)alkyl; U is O, S, NH, or N((C₁-C₂₄)alkyl); b is a keto-enol tautomer unsaturation; and n is an integer selected from 1, 2, 3, and 4; R₄ is absent, or selected from II, and -L-T; R₅ is absent, or selected from II, and -L-T; at least one of R₁, R₂, R_{N1}, R₄ and R₅ is -L-T; only one of R_{N1}, R₄ and R₅ is -L-T; each -L- is independently a linker that is selected from a bond, —O—, —S—, —NH—, —N(alkyl)-, —C(O)—, —O(c═O)—, —C(═O)O—, —C(═S)O, —C(═S)—, and —P(O)₂—; and each -T is independently selected from a therapeutic agent, a therapeutic agent pro-drug, or a therapeutic agent precursor, wherein the concentration of the therapeutic agent is enhanced in the target organ. In some embodiments, n=1, and R₁═R₂═CH₃.

In various embodiments, the linker is a bond. In other embodiments, the linker is —C(O)—. In other embodiments, the linker is —C(═S)—. In still other embodiments, the linker is —P(O)₂—. In yet other embodiments, the linker is an ether, sulfide, or an amine. In some embodiments, the linker is —O(C═O)—, or in other embodiments, —C(═O)O—.

In some embodiments of the method, the conjugate has R_{P1} is H or F; R_{P2} is H or F; R_{V1} is H or F; R_{V2} is H or F; wherein at least one of R_{P1}, R_{P2}, R_{V1}, and R_{V2} is fluorine. In another embodiment of the method, the conjugate has R_{P1} is H or F; R_{P2} is H or F; R_{V1} is H or F; R_{V2} is H or F: wherein at least one of R_{P1}, R_{P2}, R_{V1}, and R_{V2} is fluorine; R₁ is methyl; R₂ is methyl; R_{N1} is H; R_{N2} is H; and R_{N3} is absent.

In some embodiments of the method, the conjugate administered has each -T therapeutic agent, therapeutic agent pro-drug, or therapeutic agent precursor is independently —V—W—X—Y—Z, wherein V is bond, —O—, or —NH—; W is —(C₀-C₆)alkyl-, —(C₂-C₆)alkenyl-; or —(C₂-C₆)alkynyl-; X is a bond, —O—, —NH—, —CO—, —NH—(C═O)—, —SO₂—, —(C═NH)—NH—, —(C═O)—O—, or —O(C═O)—; Y is a —(C₀-C₆)alkyl-, —(C₂-C₆)alkenyl-; or —(C₂-C₆)alkynyl-; Z is a -quaternary amine, -cycloalkyl, -aryl. -heterocycle, or heteroaryl; and each nitrogen, alkyl, alkenyl, alkynyl, cycloalkyl, aryl, heterocycle, or heteroaryl is optionally substituted, wherein the concentration of the therapeutic agent is enhanced in the target organ.

In still other embodiments of the method of increasing the concentration of a therapeutic agent in a target organ is administering a therapeutically effective amount of the huperzine or huperzine analog conjugate of formula I, wherein -T is a covalently bonded glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor; a precursor to one of a glutamate receptor antagonist, an N-methyl d-aspartate receptor antagonist; mitochondrial protection, an anti-inflammatory agent, alpha-7 agonist, potassium channel blocker, sodium channel blocker, anticonvulsant, anxiolytic agent NADPH oxidase inhibitor, a gamma amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof, wherein the concentration of the therapeutic agent is enhanced in the target organ.

In still other embodiments of the method of increasing the concentration of a therapeutic agent in a target organ is administering a therapeutically effective amount of the huperzine or huperzine analog conjugate of formula I, wherein -T is —(C═O)—CH₂—CH(i-Bu)-CH₂—NH₂, wherein the concentration of the therapeutic agent is enhanced in the target organ. In other embodiments, -T is (S)—(C═O)—CH₂—CH(i-Bu)-CH₂—NH₂.

In yet other embodiments of the method of increasing the concentration of a therapeutic agent in a target organ is administering a therapeutically effective amount of the huperzine or huperzine analog conjugate of formula I, wherein -T is a covalently bonded N-methyl d-aspartate receptor antagonist selected from R-2-amino-5-phosphonopentanoate, 2-amino-7-phosphonoheptanoic acid, 3-[(R)-2-carboxypiperazin-4-yl]-prop-2-enyl-1-phosphonic acid, selfotel, amantadine, dextrallorphan, dextromethorphan, dextrorphan, dizocilpine, eticyclidine, gacyclidine, ibogaine, memantine, methoxetamine, phencyclidine, rolicyclidine, tenocyclidine, methoxydine, tiletamine, neramexane, eliprodil, etoxadrol, dexoxadrol, remacemide, delucemine, 8a-phenyldecahydroquinoline, aptiganel, rhynchophylline, ketamine, 1-aminocyclopropanecarboxylic acid, 7-chlorokynurenate, 5,7-dichlorokynurenic acid, kynurenic acid, lacosamide; a pro-drug of the NMDA receptor antagonist, a precursor of the NMDA receptor antagonist; and any combination thereof.

In still other embodiments of the method of increasing the concentration of a therapeutic agent in a target organ is administering a therapeutically effective amount of the huperzine or huperzine analog conjugate of formula I, wherein the compound is one wherein -T is any one of the structures XLXXIX-CXVIII, or pharmaceutically acceptable salt thereof:

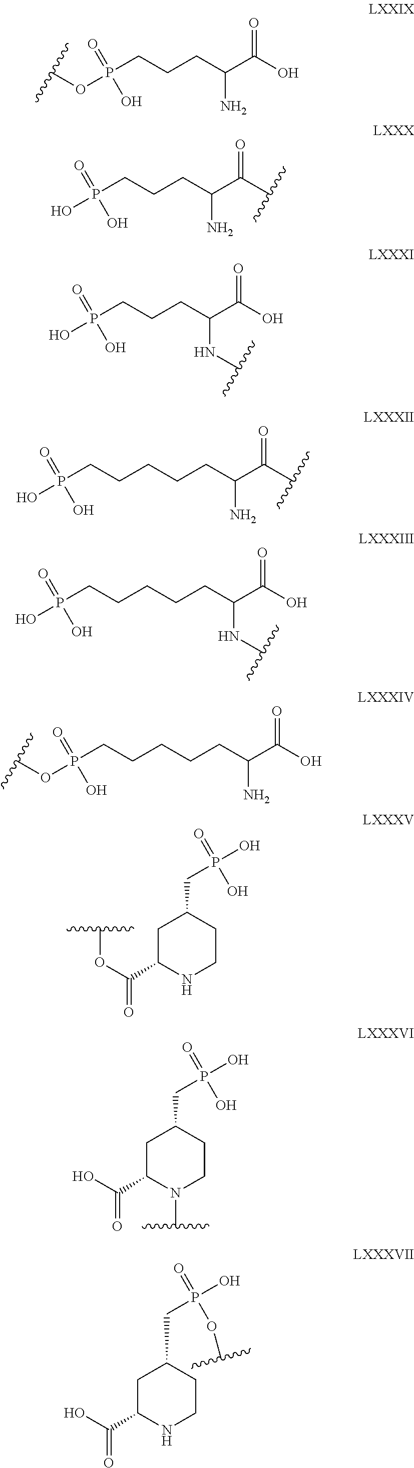

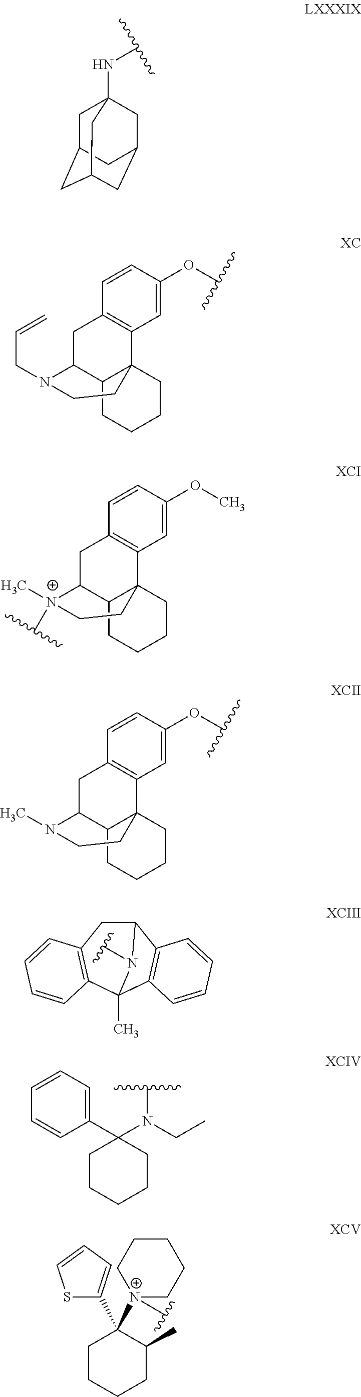

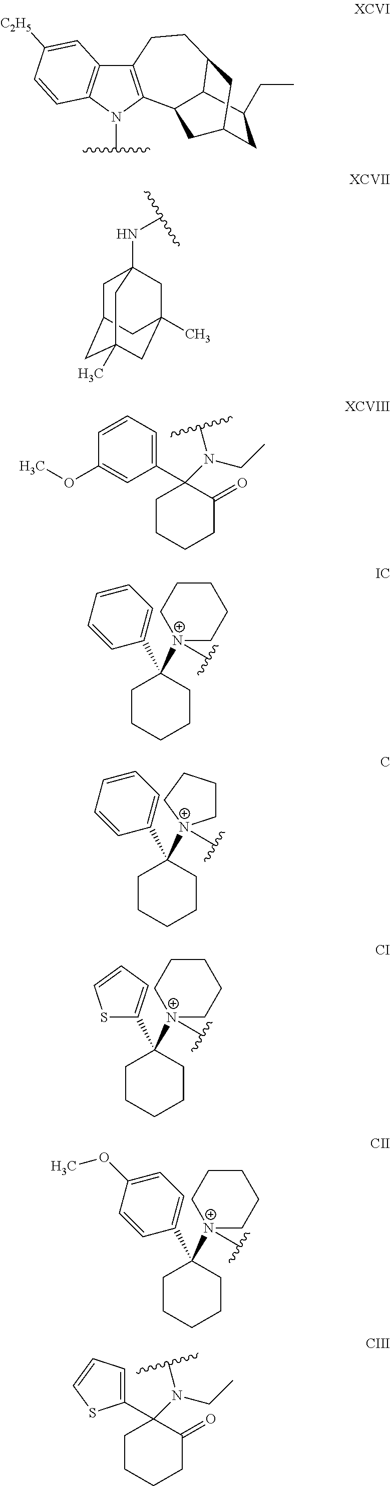

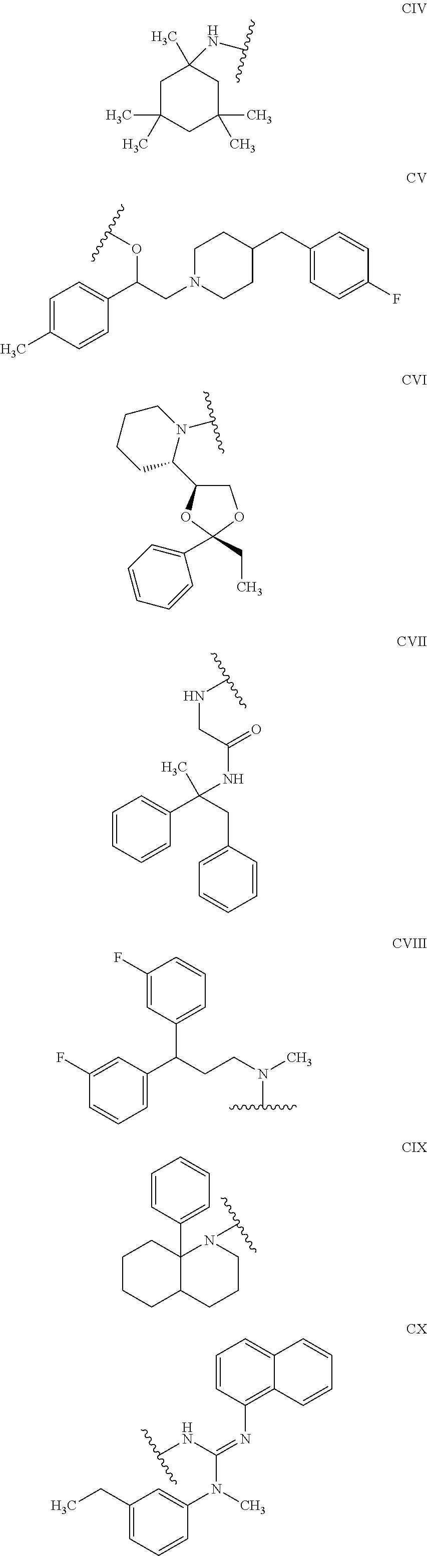

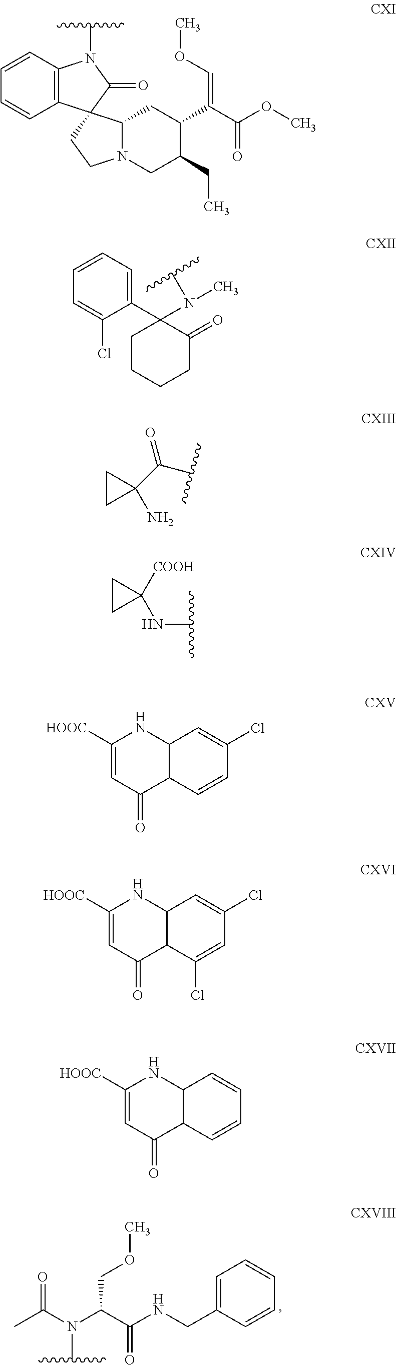

wherein the concentration of the therapeutic agent is enhanced in the target organ.

In some embodiments of the method of increasing the concentration of a therapeutic agent in a target organ is administering a therapeutically effective amount of the huperzine or huperzine analog conjugate of formula I, wherein the compound is one in which -T is a covalently bonded mitochondrial protectant selected from bethanechol and (2S,2′R,3′S,5′R)-1-methyl-2-(2-methyl-1,3-oxathiolan-5-yl)pyrrol-idine 3-sulfoxide methyl iodide; a pro-drug of the mitochondrial protectant, a precursor of the mitochondrial protectant; and any combination thereof, wherein the concentration of the therapeutic agent is enhanced in the target organ. In various embodiments, -T is any one of the structures CXIX-CXX, or pharmaceutically acceptable salt thereof:

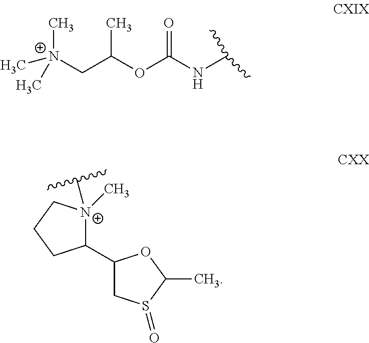 [SMILES: *NC(=O)OC(C)C[N+](C)(C)C ; *[N+]1(C)CCCC1C1CS(=O)C(C)O1]

In other embodiments of the method of increasing the concentration of a therapeutic agent in a target organ is administering a therapeutically effective amount of the huperzine or huperzine analog conjugate of formula I, wherein the compound is one in which -T is a covalently bonded anti-inflammatory agent selected from non-steroidal anti-inflammatory drugs, immune-selective anti-inflammatory derivatives, and anti-inflammatory herbal extracts, NF-Kappa B inhibitors, and EL-inhibitors; a pro-drug of the anti-inflammatory agent, a precursor of the anti-inflammatory agent; and any combination thereof, wherein the concentration of the therapeutic agent is enhanced in the target organ.

In still other embodiments of the method, the compound is one where -T is a covalently bonded antioxidant; a pro-drug to an antioxidant; or a precursor to an antioxidant. Examples of anti-oxidants which may be conjugated with huperzine or an analog thereof include, but are not limited to, ascorbic acid, glutathione, lipoic acid, uric acid, beta-carotene, vitamin A, vitamin E, co-enzyme Q; a pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (EGG), epicatechin gallate (ECG), and epigallocatechin inflate (EGCG), epigallocatechin and gallic acid, methyl gallate, and any combination thereof.

In yet other embodiments of the method, the compound is one wherein -T is the following structure or pharmaceutically acceptable salt thereof bonded through any one of NH, NH₂, COOH, OH, thiol, or an enol-tautomer oxygen of one of the following compounds: uric acid, ascorbic acid, glutathione, melatonin, alpha-tocopherol, beta-tocopherol, gamma-tocopherol, delta-tocopherol, alpha-tocotrienols beta-tocotrienol, gamma-tocotrienol, delta, tocotrienol, catechin, epicatechin, epigallocatechin, epicatechin gallate, and epigallocatechin gallate, epigallocatechin, gallic acid, methyl gallate, or a combination thereof.

In still other embodiments of the method, the compound is one where T is a covalently bonded anti-inflammatory agent; a pro-drug to an anti-inflammatory agent; or a precursor to an anti-inflammatory agent. Examples of anti-inflammatory agents which may be conjugated with huperzine or an analog thereof include, but are not limited to, compounds of non-steroidal anti-inflammatory drugs, immune-selective anti-inflammatory derivatives, anti-inflammatory herbal extracts, extracts of salix purpurea, extracts of piper longum, extracts of boswellia serrata and extracts of prunella vulgaris, NF-Kappa B inhibitors, IL-inhibitors, any combinations thereof, and/or the like. Additional examples of anti-inflammatory agents include, but are not limited to, tanshinone, cryptotanshinone, ferulic acid, cycloartenol, cycloartenyl ferulate, hydroxytyrosol, homovanillyl alcohol, 4-O-methylgallic acid, and any combination thereof:

In yet other embodiments of the method, the compound is one wherein -T is the following structure or pharmaceutically acceptable salt thereof bonded through any one of COOH or OH of one of the following compounds: ferulic acid, cycloartenol, cycloartenyl ferulate, hydroxytyrosol, homovanillyl alcohol, 4-O-methylgallic acid, and any combination thereof.

In still other embodiments of the method, the compound is one where is a covalently bonded antioxidant; a pro-drug to an antioxidant; or a precursor to an antioxidant. Examples of anti-oxidants which may be conjugated with huperzine or an analog thereof include, but are not limited to, ascorbic acid, glutathione, lipoic acid, uric acid, beta-carotene, vitamin A, vitamin E, co-enzyme Q; a pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (ECC), epicatechin gallate (ECG), and epigallocatechin gallate (EGCG), epigallocatechin and gallic acid, methyl gallate, and any combination thereof.

In yet other embodiments of the method, the compound is one wherein -T is the following structure or pharmaceutically acceptable salt thereof bonded through any one of NH, NH₂, COOH, OH, thiol, or an enol-tautomer oxygen of one of the following compounds: uric acid, ascorbic acid, glutathione, melatonin, alpha-tocopherol, beta-tocopherol, gamma-tocopherol, delta-tocopherol, alpha-tocotrienols beta-tocotrienol, gamma-tocotrienol, delta-tocotrienol, catechin, epicatechin, epigallocatechin, epicatechin gallate, and epigallocatechin gallate, epigallocatechin, gallic acid, methyl gallate, or a combination thereof.

In some embodiments of the method, a huperzine conjugate or huperzine analog conjugate is co-administered with an antioxidant. Examples of antioxidants which may be co-administered with huperzine or an analog thereof include, but are not limited to, ascorbic acid, glutathione, lipoic acid, uric acid, beta-carotene, vitamin A, vitamin E, co-enzyme Q; a pro-drug of the anti-oxidants, a precursor of the anti-oxidants; and any combination thereof, and/or the like. Additional examples of antioxidant agents include, but are not limited to, uric acid, ascorbic acid, glutathione, melatonin, tocopherols and tocotrienols (vitamin E), salubrious polyphenols, in particular catechins, the most abundant of which are epicatechin (EC), epigallocatechin (EGC), epicatechin gallate (ECG), and epigallocatechin gallate (EGCG), epigallocatechin and gallic acid, methyl gallate, and any combination thereof.

In still other embodiments oldie method, the compound is one when -T is a covalently bonded anti-inflammatory agent; a pro-drug to an anti-inflammatory agent; or a precursor to an anti-inflammatory agent. Examples of anti-inflammatory agents which may be conjugated with huperzine or an analog thereof include, but are not limited to, compounds of non-steroidal anti-inflammatory drugs, immune-selective anti-inflammatory derivatives, anti-inflammatory herbal extracts, extracts of salix purpurea, extracts of piper longum, extracts of boswellia serrata and extracts of prunella vulgaris, NF-Kappa B inhibitors, IL-inhibitors, any combinations thereof, and/or the like. Additional examples of anti-inflammatory agents include, but are not limited to, tanshinone, cryptotanshinone, ferulic acid, cycloartenol, cycloartenyl ferulate, hydroxytyrosol, homovanillyl alcohol, 4-O-methylgallic acid, and any combination thereof.

In yet other embodiments of the method, the compound is one wherein -T is the following structure or pharmaceutically acceptable salt thereof bonded through any one of COOH or OH of one of the following compounds: ferulic acid, cycloartenol, cycloartenyl ferulate, hydroxytyrosol, homovanillyl alcohol, 4-O-methylgallic acid, and any combination thereof.

In some embodiments of the method, a huperzine conjugate or huperzine analog conjugate is co-administered with an anti-inflammatory agent Examples of anti-inflammatory agents which may be co-administered with huperzine or an analog thereof include, but are not limited to, non-steroidal anti-inflammatory drugs, immune-selective anti-inflammatory derivatives, anti-inflammatory herbal extracts, NF-Kappa B inhibitors, IL-inhibitors, any combinations thereof, and/or the like. Additional examples of anti-inflammatory agents include, but are not limited to, tanshinone, cryptotanshinone, ferulic acid, cycloartenol, cycloartenyl ferulate, hydroxytyrosol, homovanillyl alcohol, extracts of salix purpurea, extracts of piper longum, 4-O-methylgallic acid, extracts of boswellia serrata and extracts of prunella vulgaris, and any combination thereof.

In some embodiments of the method of increasing the concentration of a therapeutic agent in a target organ is administering a therapeutically effective amount of the huperzine or huperzine analog conjugate of formula I, wherein -T is a covalently bonded alpha-7 agonist selected from 1,3,4-oxadiazol-2-amine, (+)—N-(1-azabicyclo[2.2.2]oct-3-yl)benzo[b]furan-2-carboxamide, AR-R17779, TC-5619, GTS-21, PHA-543,613, PNU-282,987, SSR, 180,711, tropisetron, WAY-317,538, choline, and nicotine (37-38); a pro-drug of the alpha-7 agonist; a precursor of the alpha-7 agonist; and any combination thereof, wherein the concentration of the therapeutic agent is enhanced in the target organ. In various embodiments, -T is any one of the structures XXI-XXV, XXVII-XXVIII, XXXII-XXXIV, XXXVII-XXXVIII, or pharmaceutically acceptable salt thereof:

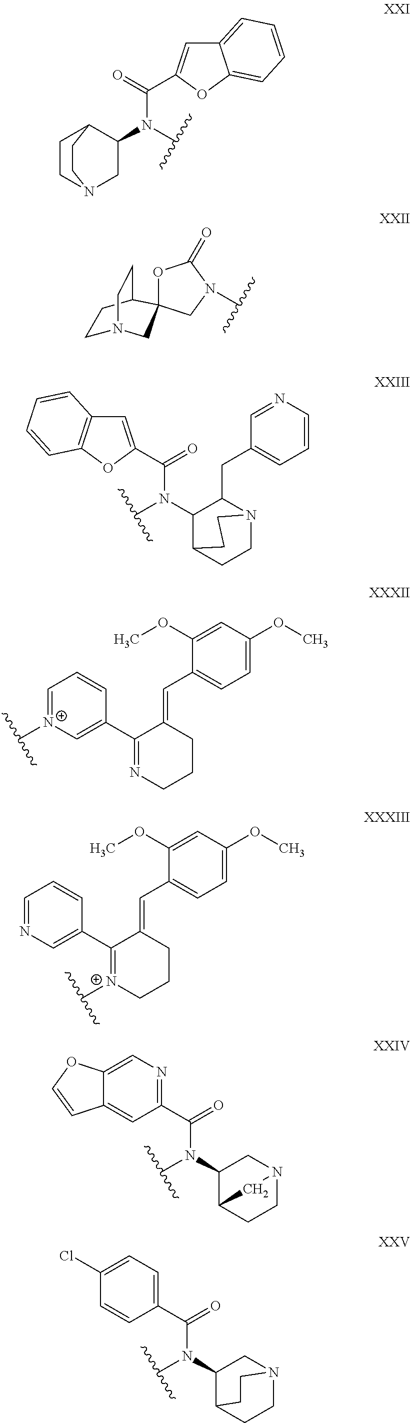

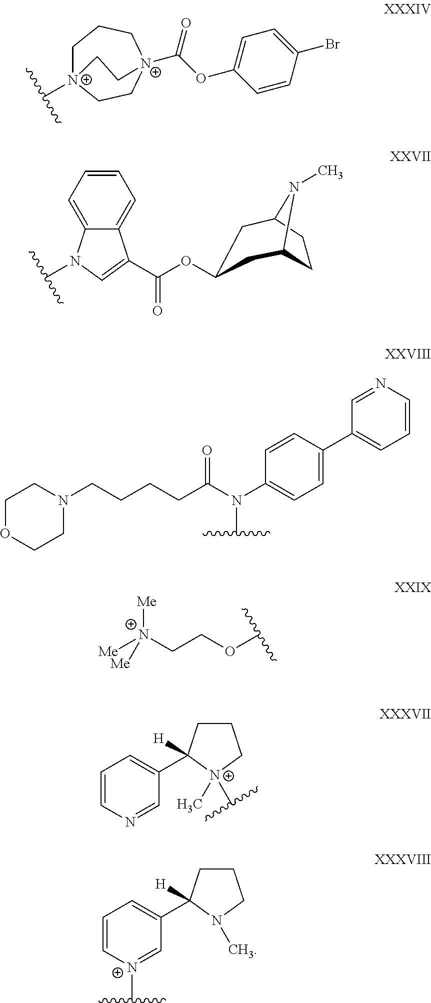

In some embodiments of the method of increasing the concentration of a therapeutic agent in a target organ is administering a therapeutically effective amount of the huperzine or huperzine analog conjugate of formula iI, wherein the compound is one in which is a covalently bound potassium channel blocker selected from dofetilide, sotalol, ibutilide, azimilide, E-4031, nifekalant, tedisamil, sematilide, 4-aminopyridine, and 3,4-diaminopyridine; a pro-drug of the potassium channel blocker; a precursor of the potassium channel blocker; and any combination thereof, wherein the concentration of the therapeutic agent is enhanced in the target organ. In various embodiments, -T is any one of the structures XXVII-XIX, or pharmaceutically acceptable salt thereof:

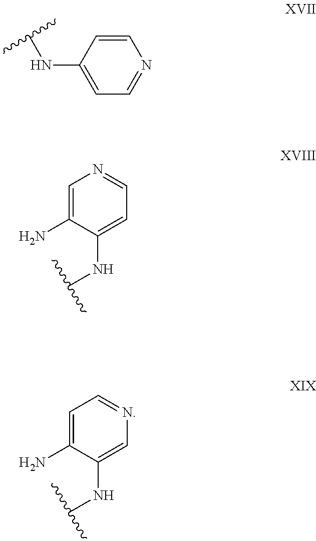 [SMILES: *Nc1ccncc1 ; *Nc1ccncc1N ; *Nc1cnccc1N]

In still other embodiments of the method of increasing the concentration of a therapeutic agent in a target organ is administering a therapeutically effective amount of the huperzine or huperzine analog conjugate of formula wherein -T is a sodium channel blocker selected from propranolol, procainamide, quinidine, disopyramide, lidocane, mexiletine, tocainide, phenytoin, encainide, flecainide, moricizine, propafenone, riluzole; a pro-drug of the sodium channel blocker; a precursor of the sodium channel blocker; and any combination thereof, wherein the concentration of the therapeutic agent is enhanced in the target organ. In yet other embodiments, -T is an anticonvulsant selected from pregabalin, (S)-pregabalin, gabapentin, stiripentol, phenobarbital, methylphenobarbital, barbexaclone, lorazepam, nitrazepam, temazepam, nimetazepam, felbamate, carbamazepine, oxcarbazepine, eslicarbazepine acetate, valproic acid, vigabatrin, progabide, tiagabine, topiramate, ethotoin, phenytoin, mephenytoin, fosphenytoin, pheneturide, beclamide, primidone, brivaracetam, levetiracetam, seletracetam, ethosuximide, acetazolamide, sultiame, phenacemide, methazolamide, zonisamide, lamotrigine; a pro-drug of the anticonvulsant, a precursor of the anticonvulsant; and any combination thereof.

In still other embodiments of the method of increasing the concentration of a therapeutic agent in a target organ is administering a therapeutically effective amount of the huperzine or huperzine analog conjugate of formula I, wherein -T is an anxiolytic agent selected from positive allosteric modulators of GABA receptor, serotonin-specific re-uptake inhibitors (SSRI), barbiturates, and benzodiazepines; a pro-drug of the anxiolytic agent, a precursor of the anxiolytic agent; and any combination thereof, wherein the concentration of the therapeutic agent is enhanced in the target organ. In yet other embodiments, -T is an anxiolytic agent selected from carisoprodol, glutethimide, meprobamate, propofol, theanine, hydroxyzine, valerenic acid, niacin, niacinamide; a pro-drug of the anxiolytic agent, a precursor of the anxiolytic agent; and any combination thereof.

In some embodiments of the method of increasing the concentration of a therapeutic agent in a target organ is administering a therapeutically effective amount of the huperzine or huperzine analog conjugate of formula I, wherein -T is an NADPH oxidase inhibitor agent selected from apocynin, a pro-drug of the NADPH oxidase inhibitor agent, a precursor of the NADPH oxidase inhibitor agent; and any combination thereof, wherein the concentration of the therapeutic agent is enhanced in the target organ. In one embodiment, -T has the structure structures XV, or pharmaceutically acceptable salt thereof:

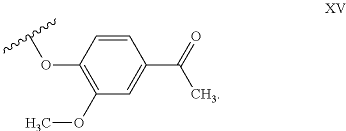 [SMILES: *Oc1ccc(C(C)=O)cc1OC]

In still other embodiments of the method of increasing the concentration of a therapeutic agent in a target organ is administering a therapeutically effective amount of the huperzine or huperzine analog conjugate of formula I, wherein -T is a gamma amino butyric acid (GABA) reuptake inhibitor agent selected from nipecotic acid; a pro-drug of the GABA reuptake inhibitor agent, a precursor of the GABA reuptake inhibitor agent; and any combination thereof, wherein the concentration of the therapeutic agent is enhanced in the target organ. In various embodiments, -T is any one of the structures XXXIX-XLI, or pharmaceutically acceptable salt thereof:

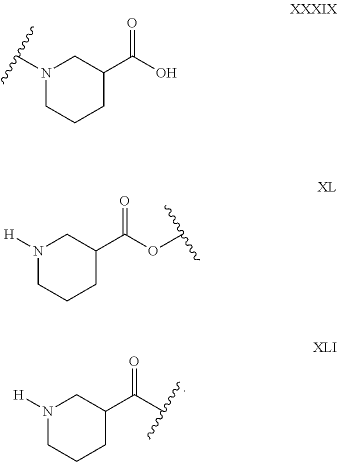 [SMILES: *N1CCCC(C(=O)O)C1 ; *OC(=O)C1CCCNC1 ; *C(=O)C1CCCNC1]

In some embodiments of the method of increasing the concentration of a therapeutic agent in a target organ is administering a therapeutically effective amount of the huperzine or huperzine analog conjugate of formula I, wherein -T is a monoamine oxidase B (MAO-B) inhibitor agent selected from lazabemide, pargyline, rasagiline, selegiline, entacapone, tolcapone, nitecapone, and quercetin; a pro-drug of the MAO-B inhibitor agent, a precursor of the MAO-B inhibitor agent; and any combination thereof, wherein the concentration of the therapeutic agent is enhanced in the target organ. In various embodiments, T is one of structures XLII-LIb, or pharmaceutically acceptable salt thereof:

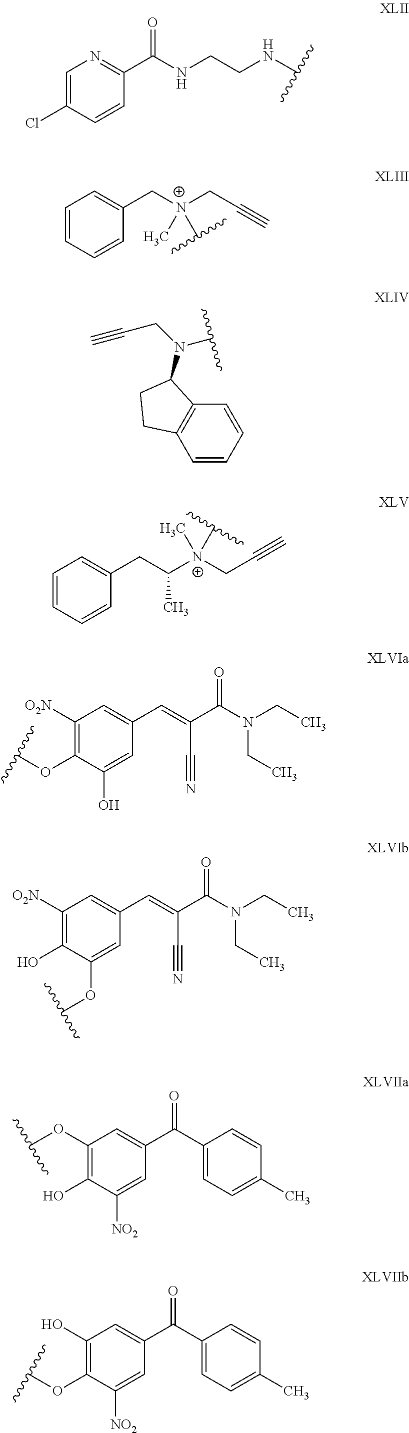

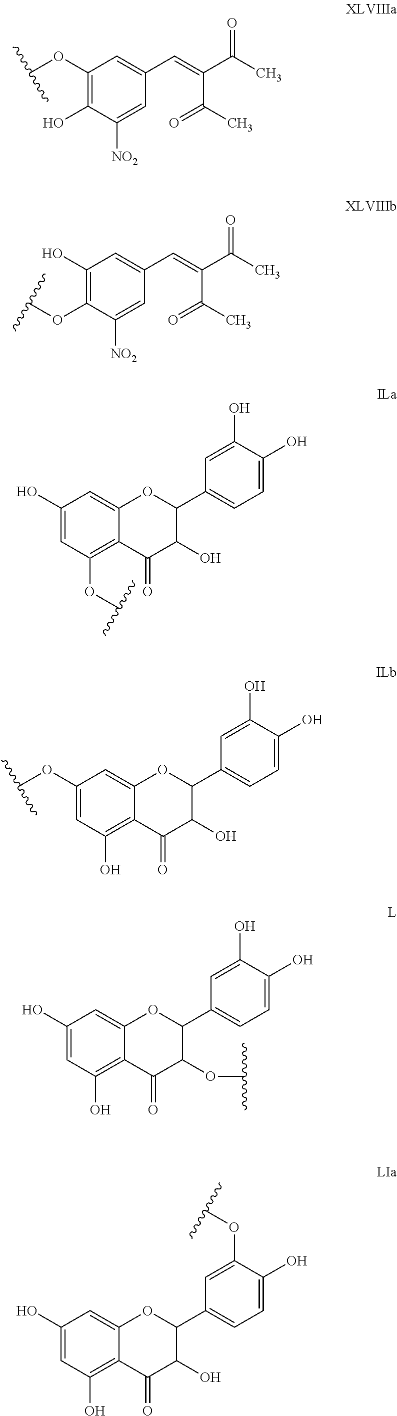 [SMILES: *Oc1cc(C=C(C(C)=O)C(C)=O)cc([N+](=O)[O-])c1O ; *Oc1c(O)cc(C=C(C(C)=O)C(C)=O)cc1[N+](=O)[O-] ; *Oc1cc(O)cc2c1C(=O)C(O)C(c1ccc(O)c(O)c1)O2 ; *Oc1cc(O)c2c(c1)OC(c1ccc(O)c(O)c1)C(O)C2=O ; *OC1C(=O)c2c(O)cc(O)cc2OC1c1ccc(O)c(O)c1 ; *Oc1cc(C2Oc3cc(O)cc(O)c3C(=O)C2O)ccc1O]

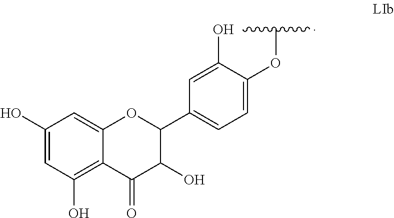 [SMILES: *Oc1ccc(C2Oc3cc(O)cc(O)c3C(=O)C2O)cc1O]

In still other embodiments of the method of increasing the concentration of a therapeutic agent in a target organ is administering a therapeutically effective amount of the huperzine or huperzine analog conjugate of formula I, wherein, -T is muscarinic receptor antagonist agent selected from atropine, cycycloverine, diphenhydramine, tolterodine, oxybutynin, opratropium, chlorpormazine, methoctramine, tripitramine, and gallamine; a pro-drug of the muscarinic receptor antagonist, a precursor of the muscarinic receptor antagonist; and any combination thereof, wherein the concentration of the therapeutic agent is enhanced in the target organ. In various embodiments, -T is any one of structures LII-LXIIf, or pharmaceutically acceptable salt thereof:

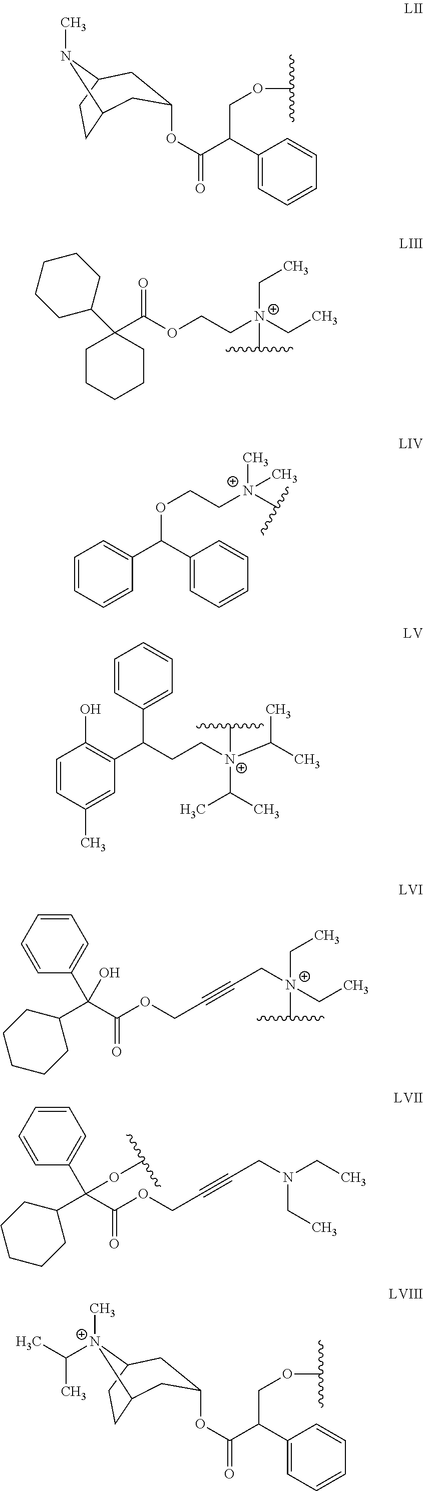

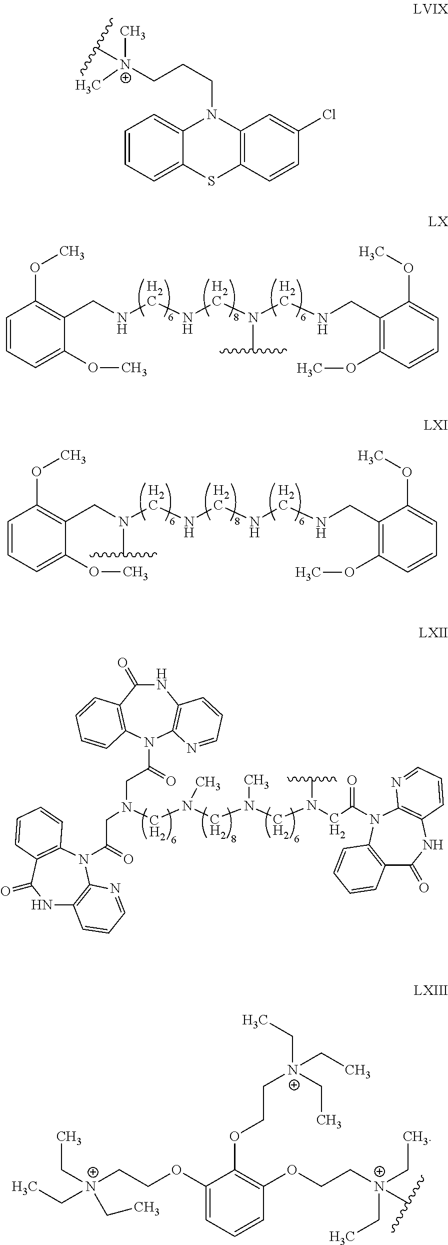

In some embodiments of the method of increasing the concentration of a therapeutic agent in a target organ is administering a therapeutically effective amount of the huperzine or huperzine analog conjugate of formula I, wherein -T is a dopamine receptor antagonist agent selected from malperone, risperidone, ziprasidone, raclopride, clozapine, haloperidol, quetiapine, domperidone, eticlopride, yohimbine, blonanserin and L-741,626 (3-[4-(4-Chlorophenyl)-4-hydroxypiperidin-1-yl]methyl-1H-indole); a pro-drug of the dopamine receptor antagonist, a precursor of the dopamine receptor antagonist, and any combination thereof wherein the concentration of the therapeutic agent is enhanced in the target organ. In various embodiments, -T is any one of structures LIX-LXXIII, or pharmaceutically acceptable salt thereof:

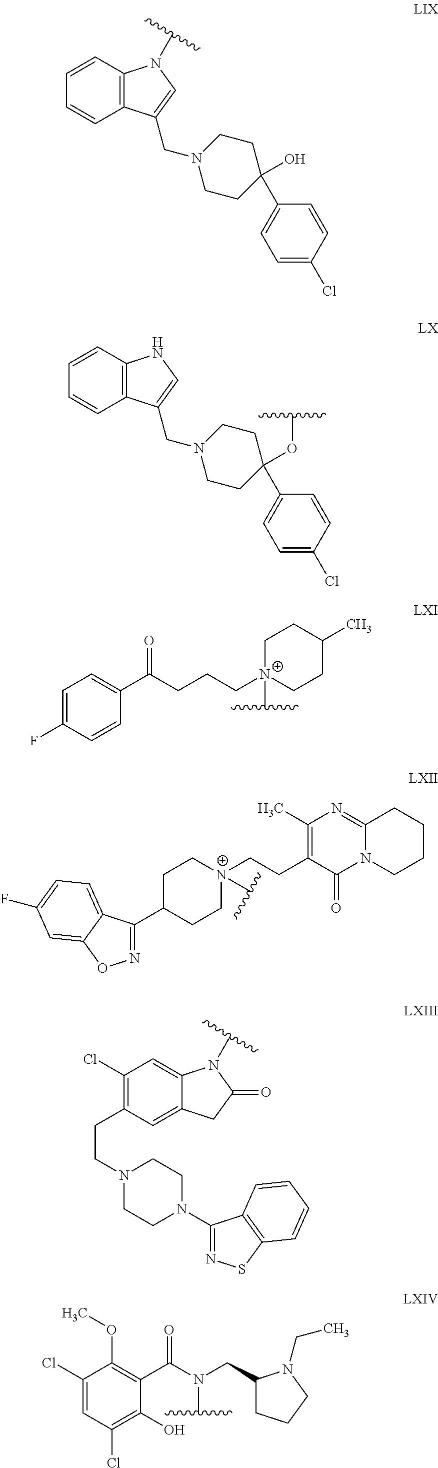

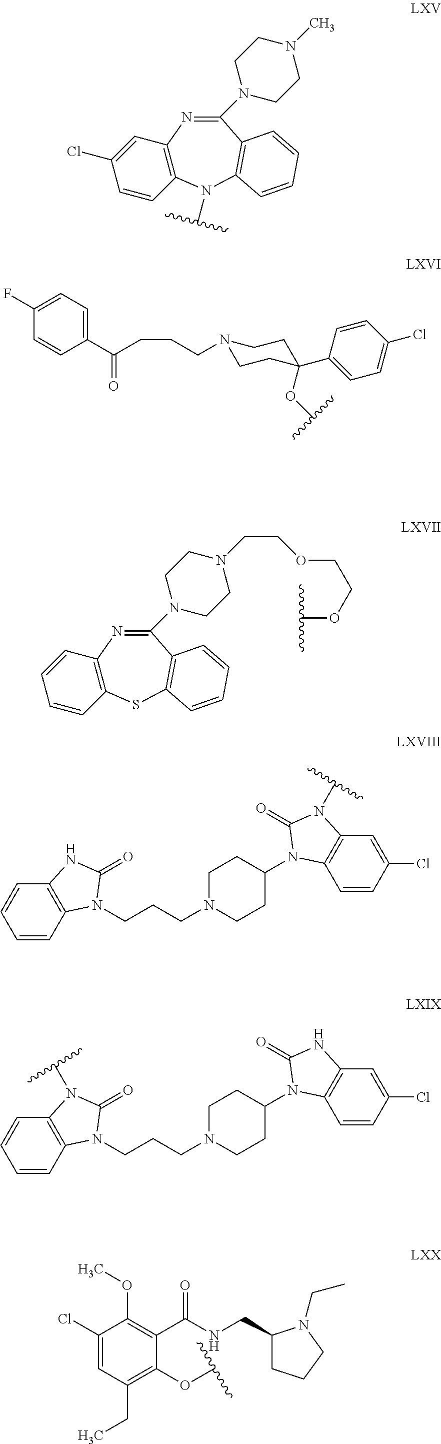 [SMILES: *N1c2ccc(Cl)cc2N=C(N2CCN(C)CC2)c2ccccc21 ; *OC1(c2ccc(Cl)cc2)CCN(CCCC(=O)c2ccc(F)cc2)CC1 ; *OCCOCCN1CCN(C2=Nc3ccccc3Sc3ccccc32)CC1 ; *n1c(=O)n(C2CCN(CCCn3c(=O)[nH]c4ccccc43)CC2)c2ccc(Cl)cc21 ; *n1c(=O)n(CCCN2CCC(n3c(=O)[nH]c4cc(Cl)ccc43)CC2)c2ccccc21 ; *Oc1c(CC)cc(Cl)c(OC)c1C(=O)NC[C@@H]1CCCN1CC]

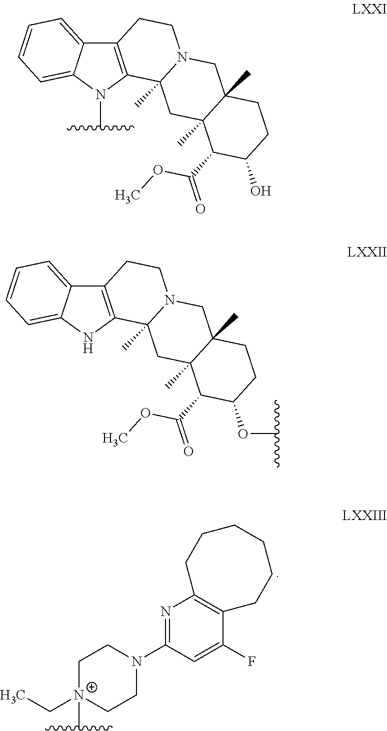 [SMILES: *n1c2c(c3ccccc31)CCN1C[C@]3(C)CC[C@H](O)[C@H](C(=O)OC)[C@@]3(C)C[C@@]21C ; *O[C@H]1CC[C@@]2(C)CN3CCc4c([nH]c5ccccc45)[C@]3(C)C[C@]2(C)[C@H]1C(=O)OC ; *[N+]1(CC)CCN(c2cc(F)c3c(n2)CCCCCC3)CC1]

In some embodiments of the method of increasing the concentration of a therapeutic agent in a target organ is administering a therapeutically effective amount of the huperzine or huperzine analog conjugate of formula I, wherein -T is a glutamate receptor (NR2B) antagonist agent selected from ifenprodil; a pro-drug of the NR2B antagonist, a precursor of the NR2B antagonist, and any combination thereof, wherein the concentration of the therapeutic agent is enhanced in the target organ. In various embodiments, -T is any one of structures LXXIV-LXXV, or pharmaceutically acceptable salts thereof:

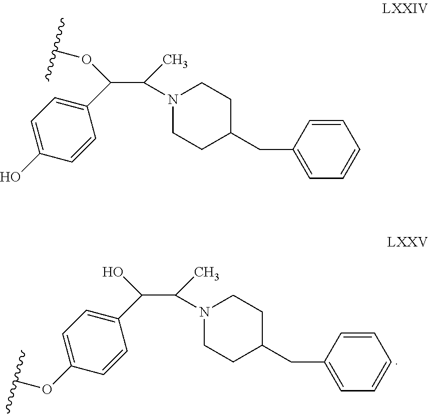 [SMILES: *OC(c1ccc(O)cc1)C(C)N1CCC(Cc2ccccc2)CC1 ; *Oc1ccc(C(O)C(C)N2CCC(Cc3ccccc3)CC2)cc1]

In some embodiments of the method of increasing the concentration of a therapeutic agent in a target organ is administering a therapeutically effective amount of the huperzine or huperzine analog conjugate of formula I, wherein -T is epigallocatechin gallate (EGCG); a pro-drug of EGCG, a precursor of EGCG, and any combination thereof. In various embodiments, -T is any one of structures LXXVIa-LXVIII, or pharmaceutically acceptable salt thereof:

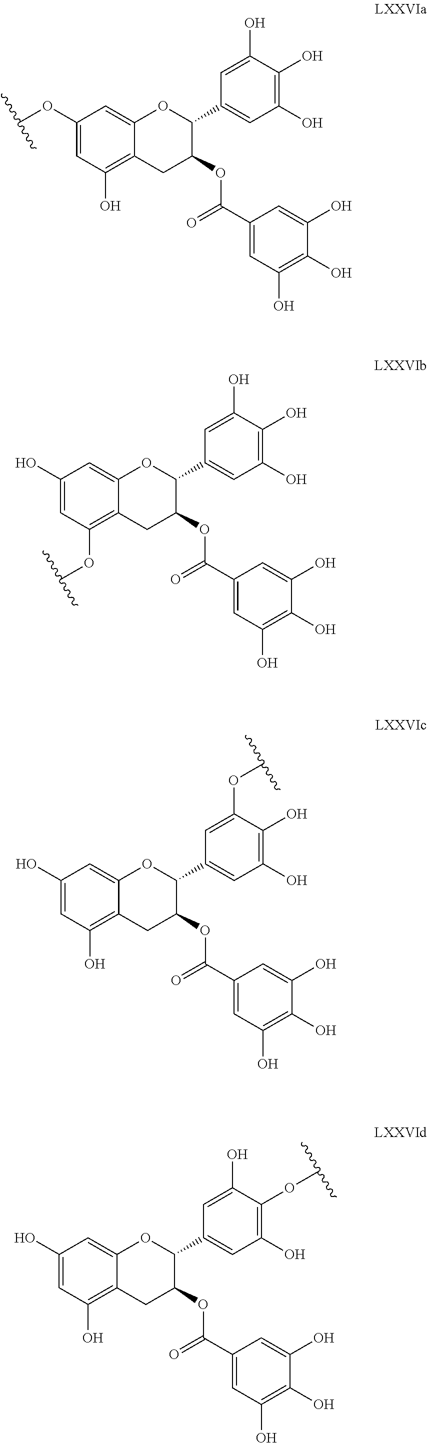 [SMILES: *Oc1cc(O)c2c(c1)O[C@H](c1cc(O)c(O)c(O)c1)[C@@H](OC(=O)c1cc(O)c(O)c(O)c1)C2 ; *Oc1cc(O)cc2c1C[C@H](OC(=O)c1cc(O)c(O)c(O)c1)[C@@H](c1cc(O)c(O)c(O)c1)O2 ; *Oc1cc([C@H]2Oc3cc(O)cc(O)c3C[C@@H]2OC(=O)c2cc(O)c(O)c(O)c2)cc(O)c1O ; *Oc1c(O)cc([C@H]2Oc3cc(O)cc(O)c3C[C@@H]2OC(=O)c2cc(O)c(O)c(O)c2)cc1O]

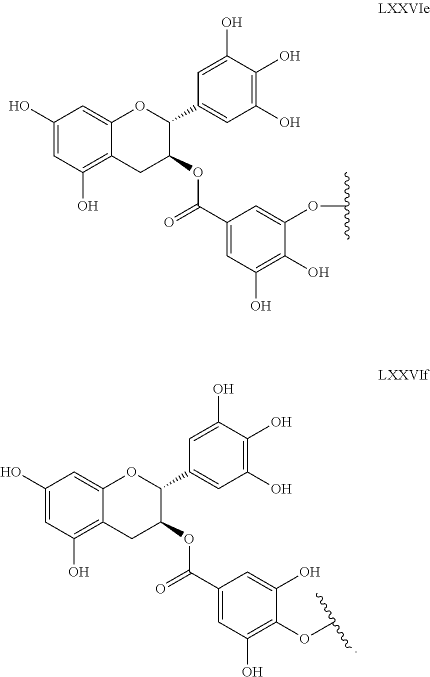 [SMILES: *Oc1cc(C(=O)O[C@H]2Cc3c(O)cc(O)cc3O[C@@H]2c2cc(O)c(O)c(O)c2)cc(O)c1O ; *Oc1c(O)cc(C(=O)O[C@H]2Cc3c(O)cc(O)cc3O[C@@H]2c2cc(O)c(O)c(O)c2)cc1O]

In still other embodiments of the method of increasing the concentration of a therapeutic agent in a target organ is administering a therapeutically effective amount of the huperzine or huperzine analog conjugate of formula I, wherein -T is an aromatase inhibitor agent selected from aminoglutethimide and formestane; a prodrug of the aromatase inhibitor, a precursor of the aromatase inhibitor, and any combination thereof, wherein the concentration of the therapeutic agent is enhanced in the target organ. In various embodiments. -T is any one of structures LXVII-LXXVIII, or pharmaceutically acceptable salt thereof:

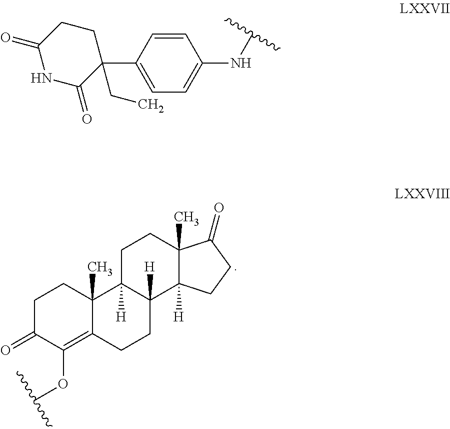

In still other embodiments of the method of increasing the concentration of a therapeutic ascent in a target organ is administering a therapeutically effective amount of the huperzine or huperzine analog conjugate of formula I, wherein -T is one of the following structures:

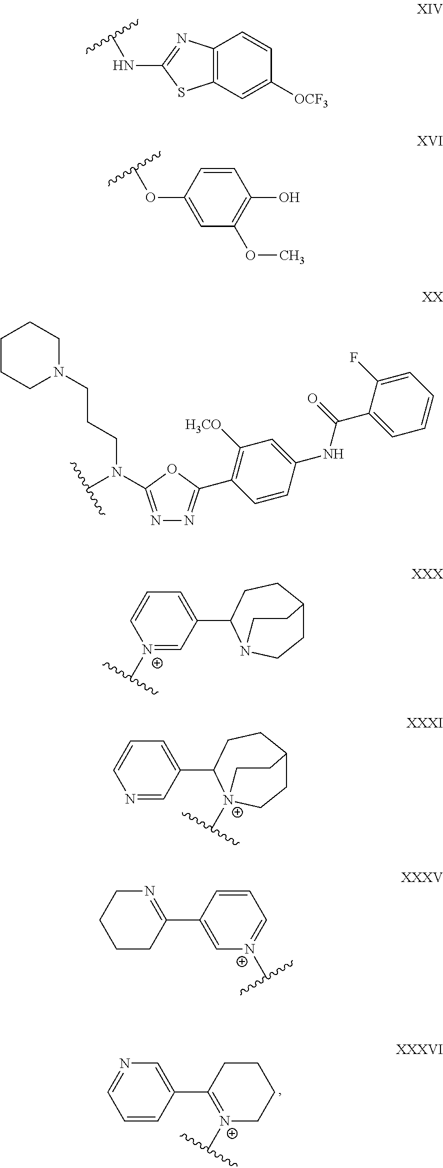

or pharmaceutically acceptable salt thereof, wherein the concentration of the therapeutic agent is enhanced in the target organ.

In still other embodiments of the method of increasing the concentration of a therapeutic agent in a target organ is administering a therapeutically effective amount of the huperzine or huperzine analog conjugate of the following structure:

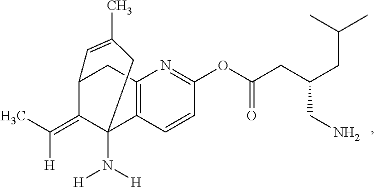 [SMILES: C/C=C1\C2C=C(C)CC1(N)c1ccc(OC(=O)C[C@@H](CN)CC(C)C)nc1C2]

or pharmaceutically acceptable salt thereof, wherein the concentration of the therapeutic agent is enhanced in the target organ.

In still other embodiments of the method of increasing the concentration of a therapeutic agent in a target organ is administering a therapeutically effective amount of the huperzine or huperzine analog conjugate of the following structure;

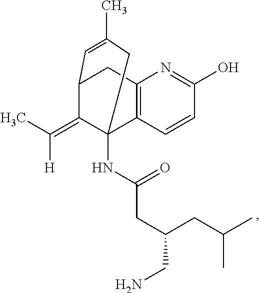 [SMILES: C/C=C1\C2C=C(C)CC1(NC(=O)C[C@@H](CN)CC(C)C)c1ccc(O)nc1C2]

or pharmaceutically acceptable salt thereof, wherein the concentration of the therapeutic agent is enhanced in the target organ.

In yet another embodiment of the method of increasing the concentration of a therapeutic agent in a target organ is administering a therapeutically effective amount of the huperzine or huperzine analog conjugate of the following structure:

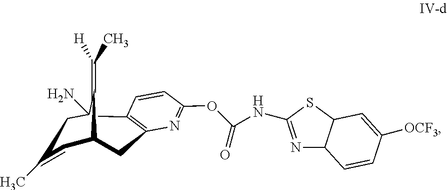

or pharmaceutically acceptable salt thereof, wherein the concentration of the therapeutic agent is enhanced in the target organ.

In still other embodiments of the method of increasing the concentration of a therapeutic agent in a target organ is administering a therapeutically effective amount of the huperzine or huperzine analog conjugate of the following structure:

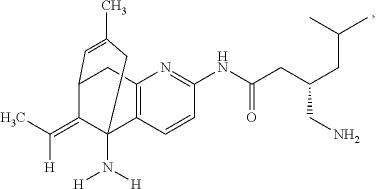 [SMILES: C/C=C1\C2C=C(C)CC1(N)c1ccc(NC(=O)C[C@@H](CN)CC(C)C)nc1C2]

or pharmaceutically acceptable salt thereof, wherein the concentration of the therapeutic agent: is enhanced in the target organ.

In still another embodiment of the method of increasing the concentration of a therapeutic agent in a target organ is administering a therapeutically effective amount of the huperzine or huperzine analog conjugate of the following structure:

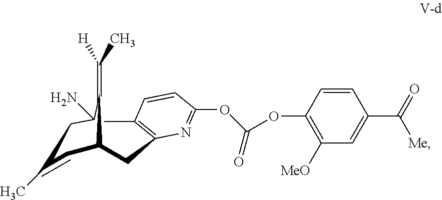

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

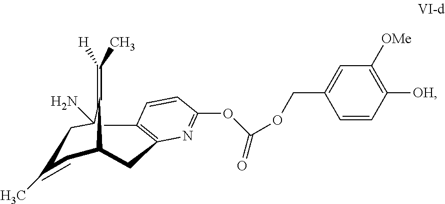

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

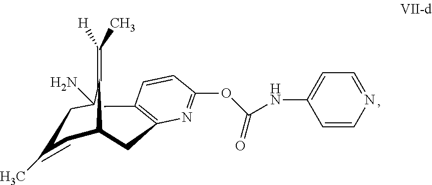

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

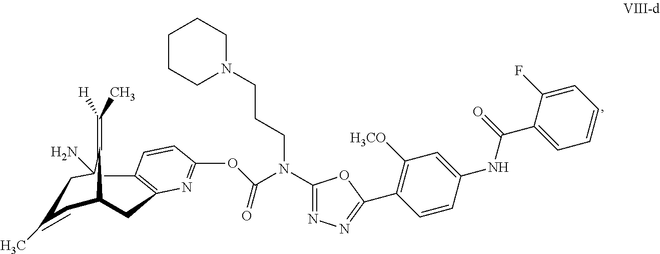

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

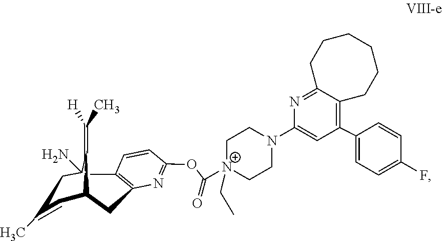

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

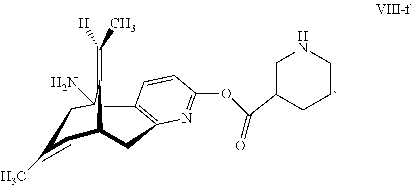

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

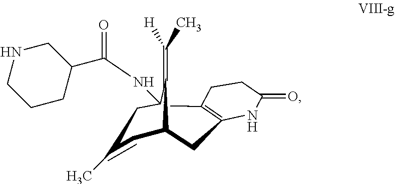

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

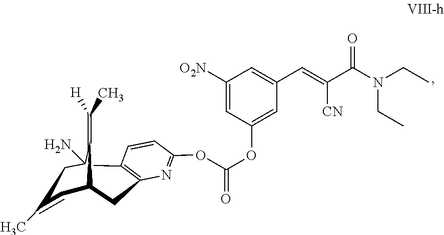

or pharmaceutically acceptable salt thereof, wherein the concentration of the therapeutic agent is enhanced in the target organ.

In still another embodiment of the method of increasing the concentration of a therapeutic agent in a target organ is administering a therapeutically effective amount of the huperzine or huperzine analog conjugate of the following structure:

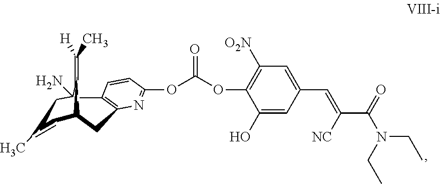

or pharmaceutically acceptable salt thereof, wherein the concentration of the therapeutic agent is enhanced in the target organ.

Other embodiments of the present invention is a compound having a general formula Ia:

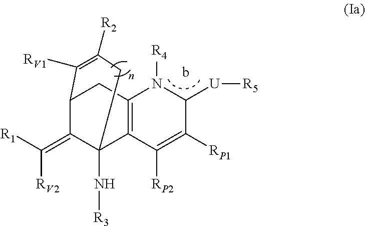

a tautomer, or pharmaceutically acceptable salt thereof, wherein R₁ is selected from H, (C₁-C₂₄)alkyl, CF₃, CF₂CF₃, CF₂CF₂CF₃, SO₂CH₃, SO₂Ph, SO₂Ar, SO₃H, and SO₃Ar, and —CH₂-L-T; R₂ is selected from H, (C₁-C₂₄)alkyl, aryl, cycloalkyl, (C₂-C₂₄)alkenyl, heterocycle, heteroaryl and —CH₂-L-T; R_{P1}, R_{P2}, R_{V1}, R_{V2} are independently selected from hydrogen and fluorine; R_{N1} is selected from H, (C₁-C₂₄)alkyl, CF₃, CF₂CF₃, CCl₃, CBr₃, CHO, and -L-T; U is O, S, NH, or N((C₁-C₂₄)alkyl); his a keto-enol tautomer unsaturation; R₃ is selected from H, CF₃, CF₂CF₃, CCl₃, CBr₃, CH₂OH, CHO, and -L-T; R₄ is absent, or selected from H, and -L-T; R₅ is absent, or selected from H, and -L-T; n is an integer selected from 1, 2, 3 and 4; and wherein -L- is a linker, -T is selected from a therapeutic agent, a therapeutic agent pro-drug, or a therapeutic agent precursor, and wherein at least one of R₁, R₂, R₃, R₄ and R₅ is -L-T. The compound is a therapeutic agent conjugated to huperzine or an analog thereof. In some embodiments, -T is selected from formulas XIV-LXXVIII. Conjugation comprises a linkage between the therapeutic agent and a site on the huperzine or an analog thereof, in some embodiments of I, L is hydrogen. In other preferred embodiments of 1, L is —(C═O)—.

An embodiment has one of R₃, R₄ and R₅ is the -L-T; R₁ is selected from CH₃, CF₃, CF₂CF₃, CF₂CF₂CF₃, SO₂CH₃, SO₂Ph, SO₂Ar, and SO₃H; and R₂ is selected from an alkyl, an aryl, a cycloalkyl, an Amyl, a heterocycle, and a heteroaryl. Another embodiment has n=1, and R₁═R₂═CH₃. An embodiment has R₂ is phenyl. Still another embodiment of the first aspect is where the linker comprises at least one of linker functional group selected from phosphoramide, phosphoester, carbonate, amide, carboxylphosphoryl anhydride, thioester, ether, thioether, amine, and ester.

In some embodiments of the method, the conjugate has R_{P1} is H or F; R_{P2} is H or F; R_{V1} is H or F; R_{V2} is H or F; wherein at least one of R_{P1}, R_{P2}, R_{V1}, and R_{V2} is fluorine. In another embodiment of the method, the conjugate has R_{P1} is H or F; R_{P2} is H or F; R_{V1} is H or F; R_{V2} is H or F; wherein at least one of R_{P1}, R_{P2}, R_{V1}, and R_{V2} is fluorine; R₁ is methyl; R₂ is methyl; and R_{N1} is H, R₃ is H.

One embodiment has R₄ as -L-T, wherein L is a bond, T is —(C═O)—CH₂—CH(i-Bu)-CH₂—NH₂, R₃ is hydrogen, R₁ is methyl, R₂, is methyl, n is 1, and R₅ is absent. Another embodiment has R₅ as -L-T, wherein L is a bond, T is —(C═O)—CH₂—CH(i-Bu)-CH₂—NH₂, R₃ is hydrogen, n is 1, R₁ is methyl, R₂ is methyl, and R₄ is absent Another embodiment has R₃ as -L-T, wherein L is a bond, T is —(C═O)—CH₂—CH(i-Bu)-CH₂—NH₂, n is 1, R₁ is methyl, R₂ is methyl, and R₄ is absent and R₅ is hydrogen or a tautomer thereof. In some embodiments given above T has an (S) chiral center.

One embodiment has R₄ as -L-T, wherein L is a bond, T is —(C═O)—(CH₂—CH(i-Bu)-CH₂—NH₂, R₃ is hydrogen, R₁ is methyl, R₂ is phenyl, n is 1, and R₅ is absent. Another embodiment has R₅ as -L-T, wherein L is a bond, T is —(C═O)—CH₂—CH(i-Bu)-CH₂—NH₂, R₃ is hydrogen, a is 1, R₁ is methyl, R₂ is phenyl, and R₄ is absent. Another embodiment has R₃ as -L-T, wherein L is a bond, T is —(C═O)—CH₂—CH(i-Bu)-CH₂—NH₂, n is 1, R₁ is methyl, R₂ is phenyl, and R₄ is absent and R₅ is hydrogen or a tautomer thereof. In some embodiments given above -T has an (S) chiral center.

One embodiment has R₄ as -L-T, wherein L is a bond, T is —(C═O)—CH═CHCO₂R₆, R₃ is hydrogen, R₁ is methyl, R₂ is methyl, n is 1, R₅ is absent, and R₆ is hydrogen or alkyl. Another embodiment has R₅ as -L-T, wherein L is a bond, T is —(C═O)—CH═CHCO₂R₆, R₃ is hydrogen, n is 1, R₁ is methyl, R₃ is methyl, and R₄ is absent, and R₆ is hydrogen or alkyl. Another embodiment has R₃ as -L-T, wherein L is a bond, is —(C═O)—CH═CHCO₂R₆, n is 1, R₁ is methyl, R₂ is methyl, and R₄ is absent and R₅ is hydrogen or a tautomer thereof, and R₆ is hydrogen or alkyl. In some embodiments given above T has an (Z) configuration. In some of the embodiments, R₆ is methyl.

One embodiment has R₄ as -L-T, wherein L is a bond, T is —(C═O)—CH═CHCO₂R₆, R₃ is hydrogen, R₁ is methyl, R₂ is phenyl, n is 1, R₅ is absent, and R₆ is hydrogen or alkyl. Another embodiment has R₃ as -L-T, wherein 1, is a bond, T is —(C═O)—CH═CHCO₂R₆, R₃ is hydrogen, n is 1, R₁ is methyl, R₂ is phenyl, and R₄ is absent, and R₆ is hydrogen or alkyl. Another embodiment has R₃ as -L-T, wherein L is a bond, T is —(C═O)—CH═CHCO₂R₆, n is 1, R₁ is methyl, R₂ is phenyl, and R₄ is absent and R₅ is hydrogen or a tautomer thereof, and R₆ is hydrogen or alkyl. In some embodiments given above T has an (Z) configuration. In some of the embodiments, R₆ is methyl.

One embodiment is a huperzine-riluzole conjugate. The embodiment may be a labile huperzine-riluzole conjugate. One embodiment has R₄ as -L-T, wherein L is a —(C═O)—, T is N-(6-(trifluoromethoxy)benzo[d]thiazol-2-ylamine), R₃ is hydrogen, R₁ is methyl, R₂ is methyl, n is 1, and R₅ is absent. Another embodiment has R₅ as -L-T, wherein L is a —(C═O)—, T is N-(6-(trifluoromethoxy)benzo[d]thiazol-2-ylamine), R₃ is hydrogen, n is 1, R₁ is methyl, R₂ is methyl, and R₄ is absent, and R₆ is hydrogen or alkyl. Another embodiment has R₃ as -L-T, wherein L is a —(C═O)—, T is N-(6-(trifluoromethoxy)benzo[d]thiazol-2-ylamine), n is 1, R₁ is methyl, R₂ is methyl, R₄ is absent, and R₅ is hydrogen or a tautomer thereof.

One embodiment is a huperzine analog-riluzole conjugate. The embodiment may be a labile huperzine analog-riluzole conjugate. One embodiment has R₄ as -L-T, wherein L is a —(C═O)—, T is N-(6-(trifluoromethoxy)benzo[d]thiazol-2-ylamine), R₃ is hydrogen, R₁ is methyl, R₂ is phenyl, n is 1, R₅ is absent. Another embodiment has R₅ as L-T, wherein L is a —(C═O)—, T is N-(6-(trifluoromethoxy)benzo[d]thiazol-2-ylamine), R₃ is hydrogen, n is 1, R₁ is methyl, R₂ is phenyl, and R₄ is absent, and R₆ is hydrogen or alkyl. Another embodiment has R₃ as -L-T, wherein L is a —(C═O)—, T is N-(6-(trifluoromethoxy)benzo[d]thiazol-2-ylamine), n is 1, R₁ is methyl, R₂ is phenyl, and R₄ is absent, and R₅ is hydrogen or a tautomer thereof.

One embodiment is a huperzine-apocynin conjugate. The embodiment may be a labile huperzine-apocynin conjugate. One embodiment has R₄ as -L-T, wherein L is a bond, T is O-(1-(4-hydroxy-3-methoxyphenyl)ethanone), R₃ is hydrogen, R₁ is methyl, R₂ is methyl, n is 1, and R₅ is absent, Another embodiment has R₅ as -L-T, wherein L is a bond, is O-(1-(4-hydroxy-3-methoxyphenyl)ethanone), R₃ is hydrogen, n is 1, R₁ is methyl, R₂ is methyl, and R₄ is absent. Another embodiment has R₃ as -L-T, wherein L is a bond, is O-(1-(4-hydroxy-3-methoxyphenyl)ethanone), n is 1, R₁ is methyl, R₂ is methyl, and R₄ is absent, and R₅ is hydrogen or a tautomer thereof.

One embodiment is a huperzine analog-apocynin conjugate. The embodiment may be a labile huperzine analog-apocynin conjugate. One embodiment has R₄ as -L-T, wherein L is a bond, T is O-(1-(4-hydroxy-3-methoxyphenyl)ethanone), R₃ is hydrogen, R₁ is methyl, R₂ is phenyl, n is 1, and R₅ is absent. Another embodiment has R₅ as -L-T, wherein L is a bond, T is O-(1-(4-hydroxy-3-methoxyphenyl)ethanone), R₃ is hydrogen, n is 1, R₁ is methyl, R, is phenyl, and R₄ is absent. Another embodiment has R₅ as -L-T, wherein L is a bond, T is O-(1-(4-hydroxy-3-methoxyphenyl)ethanone), n is 1, R₁ is methyl, R₂ is phenyl, R₄ is absent, and R₅ is hydrogen or a tautomer thereof.

One embodiment is a huperzine-vanillyl alcohol conjugate. The embodiment may be a labile huperzine-vanillyl alcohol conjugate. One embodiment has R₄ as -L-T, wherein L is a —(C═O)—, T is 4-oxymethyl-2-methoxyphenol, R₃ is hydrogen, R₁ is methyl, R₂ is methyl, n is 1, and R₅ is absent. Another embodiment has R₅ as -L-T, wherein L is a —(C═O)—, T is 4-oxymethyl-2-methoxyphenol, R₃ is hydrogen, n is 1, R₁ is methyl, R₂ is methyl, and R₄ is absent, Another embodiment has R₃ as -L-T, Wherein L is a —(C═O)—, T is 4-oxymethyl-2-methoxyphenol, n is 1, R₁ is methyl, R₂ is methyl, and R₄ is absent, and R₅ is hydrogen or a tautomer thereof.

One embodiment is a huperzine analog-vanillyl alcohol conjugate. The embodiment may be a labile huperzine analog-vanillyl alcohol conjugate. One embodiment has R₄ as -L-T, wherein L is a —(C═O)—, T is 4-oxymethyl-2-methoxyphenol, R₃ is hydrogen, R₁ is methyl, R₂ is phenyl, n is 1, and R₅ is absent, Another embodiment has R₅ as -L-T, wherein L is a —(C═O)—, T is 4-oxymethyl-2-methoxyphenol, R₃ is hydrogen, n is 1, R₁ is methyl, R₂ is phenyl, and R₄ is absent. Another embodiment has R₃ as -L-T, wherein L is a —(C═O)—, T is 4-oxymethyl-2-methoxyphenol, n is 1, R₁ is methyl, R₂ is phenyl, R₄ is absent, and R₅ is hydrogen or a tautomer thereof.

One embodiment is a huperzine-4-aminopyridine conjugate. The embodiment may be a labile huperzine-4-aminopyridine conjugate. One embodiment has R₄ as -L-T, wherein 1, is a —(C═O)—, T is 4-pyridylamine, R₃ is hydrogen, R₄ is methyl, R₂ is methyl, n is 1, and R₅ is absent. Another embodiment has R₅ as -L-T, wherein L is a —(C═O)—, T is 4-pyridylamine, R₃ is hydrogen, n is 1, is methyl, R₂ is methyl, and R₄ is absent, Another embodiment has R₃ as -L-T, wherein L is a —(C═O)—, T is 4-pyridylamine, n is 1, R₁ is methyl, R₂ is methyl, and R₄ is absent, and R₅ is hydrogen or a tautomer thereof.

One embodiment is a huperzine analog-4-aminopyridine conjugate. The embodiment may be a labile huperzine analog-4-aminopyridine conjugate. One embodiment has R₄ as -L-T, wherein L is a —(C═O)—, T is 4-pyridylamine, R₃ is hydrogen, R₁ is methyl, R₂ is phenyl, n is 1, and R₅ is absent. Another embodiment has R₅ as -L-T, wherein L is a —(C═O)—, T is 4-pyridylamine, R₃ is hydrogen, n is 1, R₁ is methyl, R₂ is phenyl, and R₄ is absent. Another embodiment has R₃ as -L-T, wherein L is a —(C═O)—, T is 4-pyridylamine, n is 1, R₁ is methyl, R₂ is phenyl, R₄ is absent, and R₅ is hydrogen or a tautomer thereof.

One embodiment is a huperzine-3,4-diaminopyridine conjugate. The embodiment may be a labile huperzine-3,4-diaminopyridine conjugate. One embodiment has T has the structure of one of moieties XVIII or XIX:

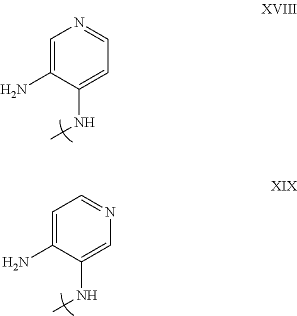

One embodiment has R₄ as -L-T, wherein L is a —(C═O)—, T is 4-(3-aminopyridyl)amine, R₃ is hydrogen, R₁ is methyl, R₂ is methyl, n is 1, and R₅ is absent. Another embodiment has R₅ as -L-T, wherein L is a —(C═O)—, T is 4-(3-aminopyridyl)amine, R₃ is hydrogen, n is 1, R₁ is methyl, R₂ is methyl, and R₄ is absent. Another embodiment has R₃ as -L-T, wherein L is a —(C═O)—, T is 4-(3-aminopyridyl)amine, n is 1, R₃ is methyl, R₂ is methyl, and R₄ is absent, and R₅ is hydrogen or a tautomer thereof. One embodiment has R₄ as -L-T, wherein L is a is 3-(4-aminopyridyl)amine, R₃ is hydrogen, R₁ is methyl, R₂ is methyl, n is 1, and R₅ is absent. Another embodiment has R₅ as -L-T, wherein L is a —(C═O)—, T is 3-(4-aminopyridyl)amine, R₃ is hydrogen, n is 1, R₁ is methyl, R₂ is methyl, and R₄ is absent. Another embodiment has R₃ as -L-T, wherein L is a —(C═O)—, T is 3-(4-aminopyridyl)amine, n is 1, R₁ is methyl, R₂ is methyl, and R is absent, and R₅ is hydrogen or a tautomer thereof.

One embodiment is a huperzine analog-3,4-diaminopyridine conjugate. The embodiment may be a labile huperzine analog-3,4-diaminopyridine conjugate. One embodiment has T has the structure of one of moieties XVIII or XIX:

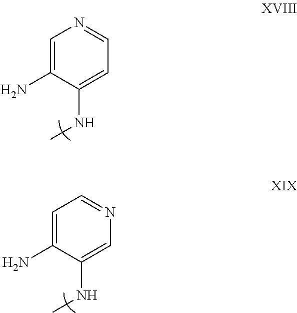

One embodiment has R₄ as -L-T, wherein L is a —(C═O)—, T is 4-(3-aminopyridyl)amine, R₃ is hydrogen, R₁ is methyl, R₂ is phenyl, n is 1, and R₅ is absent. Another embodiment has R₅ as -L-T, wherein L is a —(C═O)—, T is 4-(3-aminopyridyl)amine, R₃ is hydrogen, n is 1, R₁ is methyl, R₂ is phenyl, and R₄ is absent. Another embodiment has R₃ as wherein L is a —(C═O)—, T is 4-(3-aminopyridyl)amine, n is 1, R₁ is methyl, R₂ is phenyl, R₄ is absent, and R₅ is hydrogen or a tautomer thereof. One embodiment has R₄ as -L-T, wherein L is a —(C═O)—, T is 3-(4-aminopyridyl)amine, is hydrogen, R₁ is methyl, R₂ is phenyl, n is 1, and R₅ is absent. Another embodiment has R₅ as -L-T, wherein L is a —(C═O)—, T is 3-(4-aminopyridyl)amine, R₃ is hydrogen, n is 1, R₁ is methyl, R₂ is phenyl, and R₄ is absent. Another embodiment has R₃ as -L-T, wherein L is a —(C═O)—, T is 3-(4-aminopyridyl)amine, is 1, R₁ is methyl, R₂ is phenyl, R₄ is absent, and R₅ is hydrogen or a tautomer thereof.

One embodiment is a huperzine-1,3,4-oxadiazol-2-amine conjugate. The embodiment may be a labile huperzine-1,3,4-oxadiazol-2-amine conjugate. One embodiment has R₄ as wherein L is a —(C═O)—, T has a structure of formula XX,

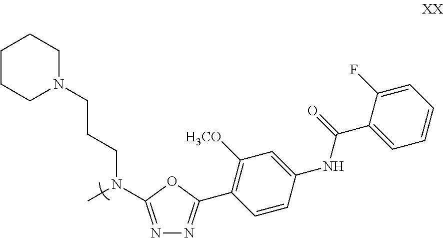 [SMILES: COc1cc(NC(=O)c2ccccc2F)ccc1-c1nnc(N(C)CCCN2CCCCC2)o1]

R₃ is hydrogen, R₁ is methyl, R₂ is methyl, n is 1, and R₅ is absent. Another embodiment has R₅ as -L-T, wherein L is a —(C═O)—, T is XX, R₃ is hydrogen, n is 1, R₁ is methyl, R₂ is methyl, and R₄ is absent. Another embodiment has R₃ as -L-T, wherein L is a —(C═O)—, T is XX, n is 1, R₁ is methyl, R₂ is methyl, and R₄ is absent, and R₅ is hydrogen or a mummer thereof.

One embodiment is a huperzine analog-1,3,4-oxadiazol-2-amine conjugate. The embodiment may be a labile huperzine analog-1,3,4-oxadiazol-2-amine conjugate, One embodiment has R₄ as -L-T, wherein L is a —(C═O)—, T is XX, R₃ is hydrogen, R₁ is methyl, R₂ is phenyl, n is 1, and R₅ is absent. Another embodiment has R₅ as -L-T, wherein L is a —(C═O)—, T is XX, R₃ is hydrogen, n is 1, R₁ is methyl, R₂ is phenyl, and R₄ is absent. Another embodiment has R₃ as -L-T, wherein L is a —(C═O)—, T is XX, n is 1, R₁ is methyl, R₂ is phenyl, R₄ is absent, and R₅ is hydrogen or a tautomer thereof.

One embodiment is a huperzine-(+)—N-(1-azabicyclo[2.2.2]oct-3-yl)benzo[b]furan-2-carboxamide conjugate. The embodiment may be a labile huperzine-(+)—N-(1-azabicyclo[2.2.2]oct-3-yl)benzo[b]furan-2-carboxamide conjugate. One embodiment has R₄ as -L-T, wherein L is a —(C═O)—, T has a structure of formula XXI,

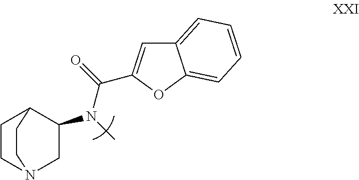 [SMILES: CN(C(=O)c1cc2ccccc2o1)[C@H]1CN2CCC1CC2]

R₃ is hydrogen, R₁ is methyl, R₂ is methyl, n is 1, and R₅ is absent. Another embodiment has R₅ as -L-T, wherein L is a —(C═O)—, T is XXI, R₃ is hydrogen, n is 1, R₁ is methyl, R₂ is methyl, and R₄ is absent. Another embodiment has R₃ as -L-T, wherein L is a —(C═O)—, T is XXI, n is 1, R₁ is methyl, R₂ is methyl, and R₄ is absent, and R is hydrogen or a tautomer thereof.

One embodiment is a huperzine analog-(+)—N-(1-azabicyclo[2.2.2]oct-3-yl)benzo[b]furan-2-carboxamide conjugate. The embodiment may be a labile huperzine analog-(+)—N-(1-azabicyclo[2.2.2]oct-3-yl)benzo[b]furan-2-carboxamide conjugate. One embodiment has R₄ as -L-T, wherein L is a —(C═O)—, T is XXI, R₃ is hydrogen, R₁ is methyl, R₂ is phenyl, n is 1, and R₅ is absent. Another embodiment has R₅ as -L-T, wherein L is a —(C═O)—, T is XXI, R₃ is hydrogen, n is 1, R₁ is methyl, R₂ is phenyl, and R₄ is absent. Another embodiment has R₃ as -L-T, wherein L is a —(C═O)—, T is XXI, n is 1, R₁ is methyl, R₂ is phenyl, R₄ is absent, and R₅ is hydrogen or a tautomer thereof.

One embodiment is a huperzine-AR-R17779 conjugate. The embodiment may be a labile huperzine-AR-R17779 conjugate. One embodiment has R₄ as -L-T, wherein L is a —(C═O)—, T has a structure of formula XXII,

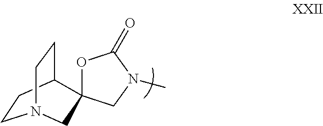

R₅ is hydrogen, R₁ is methyl, R₂ is methyl, n is 1, and R₅ is absent. Another embodiment has R₅ as -L-T, wherein L is a —(C═O)—, T is XXII, R₃ is hydrogen, n is 1, R₁ is methyl, R₂ is methyl, and R₄ is absent. Another embodiment has R₃ as -L-T, wherein L is a —(C═O)—, T is XXII, n is 1, R₁ is methyl, R₂ is methyl, and R₄ is absent, and R₅ is hydrogen or a tautomer thereof.

One embodiment is a huperzine analog-AR-R17779 conjugate. The embodiment may be a labile huperzine analog-AR-R17779 conjugate. One embodiment has R₄ as -L-T, wherein L is a —(C═O)—, T is XXII, R₃ is hydrogen, R₁ is methyl, R₂ is phenyl, n is 1, and R is absent. Another embodiment has R₅ as -L-T, wherein L is a —(C═O)—, T is XXII, R₃ is hydrogen, n is 1, R₁ is methyl, R₂ is phenyl, and R₄ is absent. Another embodiment has R₃ as -L-T, wherein L is a —(C═O)—, T is XXII, n is 1, R₁ is methyl, R₂ is phenyl, R₄ is absent, and R₅ is hydrogen or a tautomer thereof.

One embodiment is a huperzine-TC-5619 conjugate. The embodiment may be a labile huperzine-TC-5619 conjugate. One embodiment has R₄ as -L-T, wherein L is a —(C═O)—, T has a structure of formula XXIII,

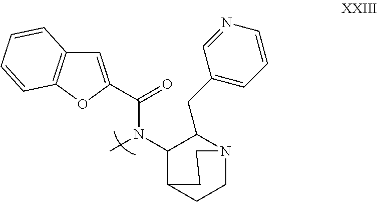 [SMILES: CN(C(=O)c1cc2ccccc2o1)C1C2CCN(CC2)C1Cc1cccnc1]

R₃ is hydrogen, R₁ is methyl, R₂ is methyl, n is 1, and R₅ is absent, Another embodiment has R₅ as -L-T, wherein L is a —(C═O)—, T is XXIII, R₃ is hydrogen, n is 1, R₁ is methyl, R₂ is methyl, and R₄ is absent. Another embodiment has R₃ as -L-T, wherein L is a —(C═O)—, T is XXIII, n is 1, R₁ is methyl, R₂ is methyl, and R₄ is absent, and R₅ is hydrogen or a tautomer thereof.

One embodiment is a huperzine analog-TC-5619 conjugate. The embodiment may be a labile huperzine analog-TC-5619 conjugate. One embodiment has R₄ as -L-T, wherein L is a —(C═O)—, T is XXIII, R₃ is hydrogen, R₁ is methyl, R₂ is phenyl, n is 1, and R₅ is absent. Another embodiment has R₅ as -L-T, wherein L is a —(C═O)—, T is XXIII R₃ is hydrogen, n is 1, R₁ is methyl, R, is phenyl, and R₄ is absent. Another embodiment has R₃ as wherein L is a —(C═O)—, T is XXIII, n is 1, R₁ is methyl, R₂ is phenyl, R₄ is absent, and R₅ is hydrogen or a tautomer thereof. At least one therapeutic agent is conjugated to the huperzine or analog thereof.

One embodiment is a huperzinc-PHA-543,613 conjugate. The embodiment may be a labile huperzine-PHA-543,613 conjugate. One embodiment has R₄ as -L-T, wherein L is a —(C═O)—, T has a structure of formula XXIV,

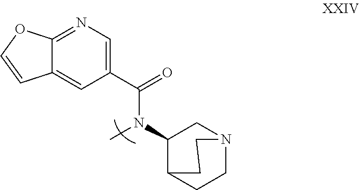 [SMILES: CN(C(=O)c1cnc2occc2c1)[C@H]1CN2CCC1CC2]

R₃ is hydrogen, R₁ is methyl, R₂ is methyl, n is 1, and R₅ is absent. Another embodiment has R₅ as -L-T, wherein L is a —(C═O)—, T is XXIV, R₃ is hydrogen, n is 1, R₁ is methyl, R₂ is methyl, and R₄ is absent. Another embodiment has R₃ as wherein L is a —(C═O)—, T is XXIV, n is 1, R₁ is methyl, R₂ is methyl, and R₄ is absent, and R₅ is hydrogen or a tautomer thereof.

One embodiment is a huperzine analog-PHA-543,613conjugate. The embodiment may be a labile huperzine analog-PHA-543,613 conjugate. One embodiment has R₄ as -L-T, wherein L is a —(C═O)—, T is XXIV, R₃ is hydrogen, R₁ is methyl, R₂ is phenyl, n is 1, and R₅ is absent. Another embodiment has R₅ as -L-T, wherein L is a —(C═O)—, T is XXIV, R₃ is hydrogen, n is 1, R₁ is methyl, R₂ is phenyl, and R₄ is absent. Another embodiment has R₃ as -L-T, wherein L is a —(C═O)—, T is XXIV, n is 1, R₁ is methyl, R₂ is phenyl, R₄ is absent, and R₅ is hydrogen or a tautomer thereof. At least one therapeutic agent is conjugated to the huperzine or analog thereof.

One embodiment is a huperzine-PNU-253,987 conjugate. The embodiment may be a labile huperzine-PNU-282,987 conjugate. One embodiment has R₄ as -L-T, wherein L is a —(C═O)—, T has a structure of formula XXV,

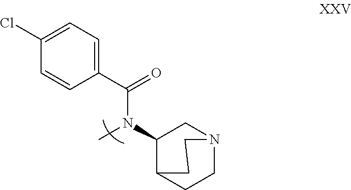 [SMILES: CN(C(=O)c1ccc(Cl)cc1)[C@H]1CN2CCC1CC2]

R₃ is hydrogen, R₁ is methyl, R₂ is methyl, n is 1, and R₅ is absent. Another embodiment has R₅ as -L-T, wherein L is a —(C═O)—, T is XXV, R₃ is hydrogen, n is 1, R₁ is methyl, R₂ is methyl, and R₄ is absent. Another embodiment has R₃ as wherein L is a —(C═O)—, T is XXV, n is 1, R₁ is methyl, R₂ is methyl, and Its is absent, and R₃ is hydrogen or a tautomer thereof.

One embodiment is a huperzine analog-PNU-282,987 conjugate. The embodiment may be a labile huperzine analog-PNU-282,987 conjugate. One embodiment has R₄ as -L-T, wherein L is a —(C═O)—, T is XXV, R₃ is hydrogen. R₃ is methyl, R is phenyl, n is 1, and R₅ is absent. Another embodiment has R₅ as -L-T, wherein L is a —(C═O)—, T is XXV, R₃ is hydrogen, n is 1, R₁ is methyl, R₂ is phenyl, and R₁ is absent. Another embodiment has R₃ as -L-T, wherein L is a —(C═O)—, T is XXV, n is 1, R₁ is methyl, R₂ is phenyl, R₄ is absent, and R₃ is hydrogen or a tautomer thereof. At least one therapeutic agent is conjugated to the huperzine or analog thereof.

The site of conjugation to the huperzine or an analog thereof is on at least one of R₁, R₂, R₃, R₄ or R₅ of structure (I). In some, R₄ is absent and R₅ is a site of conjugation to the therapeutic agent, or R₄ is a site of conjugation to the therapeutic agent and R₅ is absent. In other, structure (I) is conjugated with a therapeutic agent on only one of R₁, R₂, R₃, R₄ or R₅.

One embodiment is a huperzine-PHA-709829 conjugate. The embodiment may be a labile huperzine-PHA-709829 conjugate. One embodiment has R₄ as wherein L is a —(C═O)—, T has a structure of formula XXVI,

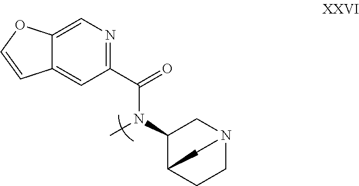 [SMILES: CN(C(=O)c1cc2ccoc2cn1)[C@H]1CN2CC[C@H]1C2]

R₃ is hydrogen, R₁ is methyl, R₂ is methyl, n is 1, and R₅ is absent Another embodiment has R₅ as -L-T, wherein L is a —(C═O)—, T is XXVI, R₃ is hydrogen, n is 1, R₁ is methyl, R₂ is methyl, and R₄ is absent. Another embodiment has R₃ as -L-T, wherein L is a —(C═O)—, T is XXVI, n is 1, R₁ is methyl, R₂ is methyl, and R₄ is absent, and R₅ is hydrogen or a tautomer thereof.

One embodiment is a huperzine analog-PHA-709829 conjugate. The embodiment may be a labile huperzine analog-PHA-709829 conjugate. One embodiment has R₄ as -L-T, wherein L is a —(C═O)—, T is XXVI, R₃ is hydrogen, R₁ is methyl, R₂ is phenyl, a is 1, and R₅ is absent. Another embodiment has R₅ as -L-T, wherein L is a —(C═O)—, T is XXVI, is hydrogen, n is 1, R₁ is methyl, R₂ is phenyl, and R₄ is absent. Another embodiment has R₃ as -L-T, wherein L is a —(C═O)—, T XXVI, n is 1, R₁ is methyl, R₂ is phenyl, R₄ is absent, and R₅ is hydrogen or a tautomer thereof. At least one therapeutic agent is conjugated to the huperzine or analog thereof.

One embodiment is a huperzine-tropisetron conjugate. The embodiment may be a labile huperzine-tropisetron conjugate. One embodiment has R₄ as -L-T, wherein L is a —(C═O)—, T has a structure of formula XXVII,

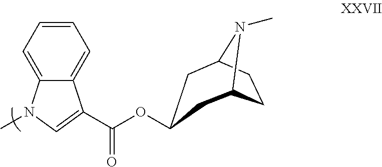

R₃ is hydrogen, R₁ is methyl, R₂ is methyl, n is 1, and R₅ is absent. Another embodiment has R₅ as -L-T, wherein L is a —(C═O)—, T is XXVII, R₃ is hydrogen, n is 1, R₁ is methyl, R₂ is methyl, and R₄ is absent. Another embodiment has R₃ as -L-T, Wherein L is a —(C═O)—, T is XXVII, n is 1, R₁ is methyl, R₂ is methyl, and R₄ is absent, and R₅ is hydrogen or a tautomer thereof.

One embodiment is a Imperzine analog-tropisetron conjugate. The embodiment may be a labile huperzine analog-tropisetron conjugate. One embodiment has R₄ as -L-T, wherein L is a —(C═O)—, T is XXVII, R₃ is hydrogen, R₁ is methyl, R₂ is phenyl, n is 1, and R₅ is absent. Another embodiment has R₅ as -L-T, wherein L is a —(C═O)—, T is XXVII, R₃ is hydrogen, n is 1, R₁ is methyl, R₂ is phenyl, and R₄ is absent. Another embodiment has R₃ as -L-T, wherein L is a —(C═O)—, T is XXVII, n is 1, R₁ is methyl, R₂ is phenyl, R₄ is absent, and R₅ is hydrogen or a tautomer thereof. At least one therapeutic agent is conjugated to the huperzine or analog thereof.

One embodiment is a huperzine-tropisetron conjugate. The embodiment may be a labile huperzine-tropisetron conjugate. One embodiment has R₄ as -L-T, wherein L is a —(C═O)—, T has a structure of formula XXVIII,

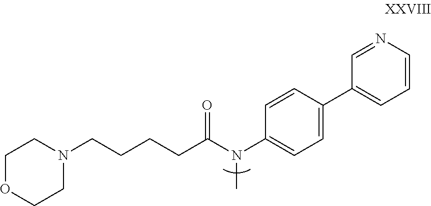 [SMILES: CN(C(=O)CCCCN1CCOCC1)c1ccc(-c2cccnc2)cc1]

R₃ is hydrogen, R₁ is methyl, R₂ is methyl, n is 1, and R₅ is absent. Another embodiment has R₅ as -L-T, wherein L is a —(C═O)—, T is XXVIII, R₃ is hydrogen, n is 1, R₁ is methyl, R₂ is methyl, and R₄ is absent. Another embodiment has R₃ as -L-T, wherein L is a —(C═O)—, T is XXVIII, n is 1, R₁ is methyl, R₂ is methyl, and R₄ is absent, and R₅ is hydrogen or a tautomer thereof.

One embodiment is a huperzine analog-tropisetron conjugate. The embodiment may be a labile huperzine analog-tropisetron conjugate. One embodiment has R₄ as -L-T, wherein L is a —(C═O)—, T is XXVIII, R₃ is hydrogen, R₁ is methyl, R₂ is phenyl, n is 1, and R₅ is absent. Another embodiment has R₅ as -L-T, wherein L is a —(C═O)—, T is XXVIII, R₃ is hydrogen, n is 1, R₁ is methyl, R₂ is phenyl, and R₄ is absent. Another embodiment has R₃ as -L-T, wherein L is a —(C═O)—, T is XXVIII, n is 1, R₁ is methyl, R₂ is phenyl, R₄ is absent, and R₅ is hydrogen or a tautomer thereof. At least one therapeutic agent is conjugated to the huperzine or analog thereof.

One embodiment is a huperzine-choline conjugate. The embodiment may be a labile huperzine-choline conjugate. One embodiment has R₄ as -L-T, wherein L is a —(C═O)—, T has a structure of formula XXIX,

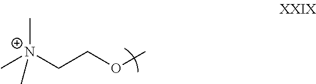

R₃ is hydrogen, R₁ is methyl, R₂ is methyl, n is 1, and R₅ is absent. Another embodiment has R₅ as -L-T, wherein L is a —(C═O)—, T is XXIX, R₃ is hydrogen, n is 1, R₁ is methyl, R₂ is methyl, and R₄ is absent. Another embodiment has R as -L-T, wherein L is a —(C═O)—, T is XXIX, n is 1, R₁ is methyl, R₂ is methyl, and R₄ is absent, and R₅ is hydrogen or a tautomer thereof.

One embodiment is a huperzine analog-choline conjugate. The embodiment may be a labile huperzine analog-choline conjugate. One embodiment has R₄ as -L-T, wherein L is a —(C═O)—, T is XXIX, R₁ is hydrogen, R₁ is methyl, R₂ is phenyl, n is 1, and R₅ is absent. Another embodiment has R₅ as -L-T, wherein L is a —(C═O)—, T is XXIX, R₃ is hydrogen, n is 1, R₁ is methyl, R₂ is phenyl, and R₄ is absent. Another embodiment has R₃ as -L-T, wherein L is a —(C═O)—, T is XXIX, n is 1, R₁ is methyl, R₂ is phenyl, R₄ is absent, and R₅ is hydrogen or a tautomer thereof. At least one therapeutic agent is conjugated to the huperzine or analog thereof.

One embodiment is a huperzine-choline conjugate. The embodiment may be a labile huperzine-choline conjugate.

One embodiment of formula I has one of R₃, R₄, and R₅ as -L-T, wherein -T has a structure of any one of formula XXX-LXXVILI. An embodiment has R₄ as -L-T, n is 1, R₁ is methyl, R₂ is methyl, and R₅ is absent. Another embodiment has R₅ as -L-T, wherein L is a —(C═O)—, T has a structure of any one of formula XXX-LXXVIII, n is 1, R₁ is methyl, R₂ is methyl, and R₄ is absent. Another embodiment has R₃ as -L-T, wherein L is a T has a structure of any one of formula XXX-LXXVIII, n is 1, R₁ is methyl, R₂ is methyl, and R₄ is hydrogen, R₅ is absent or tautomer thereof.

One embodiment is a huperzine analog-choline conjugate. The embodiment may be a labile huperzine analog-choline conjugate. One embodiment has R₄ as -L-T, wherein L is as defined above, T has a structure of any one of formula XXX-LXXVIII, R₃ is hydrogen, R₁ is methyl, R₂ is phenyl, n is 1, and R₅ is absent. Another embodiment has R₅ as -L-T, T has a structure of any one of formula XXX-LXXVIII, R₃ is hydrogen, n is 1, R₁ is methyl, R₂ is phenyl, and R₄ is absent. Another embodiment has R₃ as -L-T, T has a structure of any one of formula XXX-LXXVIII, n is 1, R₁ is methyl, R₂ is phenyl, R₄ is absent, and R₅ is hydrogen or a tautomer thereof. At least one therapeutic agent is conjugated to the huperzine or analog thereof.

In further variations of each one of the above embodiments of analogs of huperzine and conjugates thereof, n is 1. Similarly, in other variations of each one of the above embodiments of analogs of huperzine and conjugates thereof, n is 2.

The site of conjugation to the huperzine or an analog thereof is on at least one of R₁, R₂, R₃, R₄ or R₅ of structure (Ia). In some embodiments, R₄ is absent and R₅ is a site of conjugation to the therapeutic agent, or R₄ is a site of conjugation to the therapeutic agent and R₅ is absent. In other embodiments, structure (I) is conjugated with a therapeutic agent on only one of R₁, R₂, R₃, R₄ or R₅.

For the purposes of this invention, the term “linker” is intended to encompass any chemical entity that links the therapeutically active compound and the huperzine or huperzine analog. In embodiments with a plurality of linkers, the linkers may differ. In some embodiments, the linker, -L-, is a bond in some embodiments, the conjugation is a labile covalent bond. In other embodiments the linkage is a robust covalent bond. In other embodiments, the linker is a linker functional group. In still other embodiments, the linker is a linker moiety comprising a first end and a second end, each end of the linker comprising a functional group. Linker moieties, as described herein, include, but are not limited to aminohexanoic acid, polyglycine, polyamides, polyethylenes, and short functionalized polymers having a carbon backbone which is from one to about twelve carbon molecules in length. Such linkers may be designed to facilitate, influence, modulate or regulate the release of the therapeutically active compound at the desired target site. Such linkers may also facilitate enzymatic release at certain intracellular sites.

The term linker “functional group” is defined herein as any functional group for covalently binding the huperzine or huperzine analog to the linker moiety or therapeutically active agent or the linker moiety to the therapeutically active agent. These groups can be designated either “weak” or “strong” based on the stability of the covalent bond which the linker functional group will form between the linker and either the huperzine or huperzine analog or therapeutically active agent. The weak functionalities include, but are not limited to phosphoramide, phosphoester, carbonate, amide, carboxylphosphoryl anhydride, thioester and most preferably ester. The strong functionalities include, but are not limited to ether, thioether, amine, amide and most preferably ester. The use of a strong linker functional group will tend to decrease the rate at which the compounds will be released at the target site, whereas the use of a weak linker functional group between the linker moiety and the compounds may act to facilitate release of the compounds at the target site, Enzymatic release is, of course, also possible, but such enzyme-mediated modes of release will not necessarily be correlated with bond strength in such embodiments of the invention. Linkers comprising enzyme active site recognition groups, such as groups comprising peptides having proteolytic cleavage sites therein, are envisioned as being within the scope of the present invention.

The conjugates of the invention are may comprise linkers that impart differential release properties on the conjugates related to differential expression or activity of enzymatic activities in physiologically restricted or protected sites in comparison with such activities in systemic circulation or in inappropriate targets, such as hepatic, renal or hematopoietic tissues. Differential release is also provided in certain embodiments in specific cell types comprising such physiologically protected tissues.

In some embodiments of the present invention are conjugates of huperzine or huperzine analogs. The conjugates provide huperzine, huperzine analog, conjugate, therapeutic agent, therapeutic agent pro-drug, and/or therapeutic agent precursor in a specific delivery to brain tissue for the alleviation or amelioration of pathological disease states in the brain. Thus, the present invention provides methods and compositions of matter for facilitating the transit of such conjugates of psychotropic, neurotropic or neurological drugs, agents and compounds across the blood-brain barrier and into targeted regions of the brain, for the treatment of animal, preferably human, diseases and pathological conditions.

One embodiment provides huperzine conjugates and huperzine analog conjugates of a glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor; a precursor to one of a glutamate receptor antagonist, an N -methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor a gamma amino butyric acid (PARA) reuptake inhibitor, a monoamine oxidase B (MAO-B) inhibitor, a muscarinic receptor antagonist, a dopamine receptor antagonist, a glutamate receptor (NR2B) antagonist, epigallocatechin gallate, aromatase inhibitor; and any combination thereof, and/or the like.

One embodiment provides huperzine conjugates and huperzine analog conjugates of an N-methyl-d-aspartate receptor antagonist (NMDA receptor antagonist). Examples of NMDA receptor antagonists which may be conjugated to huperzine or an analog thereof include, but are not limited to, R-2-amino-5-phosphonopentanoate, 2-amino-7-phosphonoheptanoic acid, 3-[(R)-2-carboxypiperazin-4-yl]prop-2-enyl-1-phosphonic acid, selfotel, amantadine, dextrallorphan, dextromethorphan, dextrorphan, dizocilpine ethanol, eticyclidine, gacyclidine, ibogaine, magnesium, memantine, methoxetamine, nitrous oxide, phencyclidine, rolicyclidine, tenocyclidine, methoxydine, tiletamine, xenon, neramexane, eliprodil, etoxadrol, dexoxadrol, remacemide, delucemine, 8a-phenyldceahydroquinoline, aptiganel, remacemide, rhynchophylline, ketamine, 1-aminocyclopropanecarboxylic acid, 7-chlorokynurenate, 5,7-dichlorokynurenic acid, kynurenic acid, lacosamide; a pro-drug of the NMDA receptor antagonist, a precursor of the NMDA receptor antagonist; and any combination thereof, and/or the like.

One embodiment provides huperzine conjugates and huperzine analog conjugates of a mitochondrial protectant, a pro-drug of the mitochondrial protectant, precursor to the mitochondrial protectant, combinations thereof, and the like. Mitochondrial protectant therapeutic agents include, but are not limited to, muscarinic receptor agonists that activate M2 subtype to prevent ACh release which in turn can activate mitochondrial protection. Muscarinic receptor agonists include, but are not limited to, compounds such as bethanechol and (2S,2′R,3′S,5′R)-1-methyl-2-(2-methyl-1,3-oxathiolan-5-yl)pyrrol-idine 3-sulfoxide methyl iodide, any combinations thereof, and/or the like.

Further, embodiments provides huperzine conjugates and huperzine analog conjugates of therapeutic agents including an anti-inflammatory agent, a pro-drug of the anti-inflammatory anent, a precursor of the anti-inflammatory agent, or combinations thereof. Examples of anti-inflammatory agents which may be conjugated to huperzine or an analog thereof include, but are not limited to, non-steroidal anti-inflammatory drugs, immune-selective anti-inflammatory derivatives, anti-inflammatory herbal extracts, NF-Kappa B inhibitors, IL-inhibitors, any combinations thereof, and/or the like.

Further, embodiments provides huperzine conjugates and huperzine analog conjugates of therapeutic agents including an alpha-7 agonist, a pro-drug of the alpha-7 agonist, a precursor of the alpha-7 agonist, or combinations thereof. Examples of alpha-7 agonists which may be conjugated to huperzine or an analog thereof include, but are not limited to, non-steroidal anti-inflammatory drugs, immune-selective anti-inflammatory derivatives, anti-inflammatory herbal extracts, NE-Kappa B inhibitors, IL-inhibitors, 1,3,4-oxadiazol-2-amine, (+)—N-(1-azabicyclo[2.2.2]oct-3-yl)benzo[b]furan-2-carboxamide, A-582941, AR-R17779, TC-5619, GTS-21, PHA-543,613, PNU-282,987, PHA-709829, SSR-180,711, tropisetron, WAY-317,538, anabaseine, choline, and nicotine, any combinations thereof, and/or the like.

Further, embodiments provides huperzine conjugates and huperzine analog conjugates of therapeutic agents including a sodium channel blockers, a pro-drug of the sodium channel blockers, a precursor of the sodium channel blockers, or combinations thereof. Examples of sodium channel blockers which may be conjugated to huperzine or an analog thereof include, but are not limited to, saxitoxin, neossaxitoxin, tetrodotoxin, propranolol, procainamide, quinidine, disopyramide, lidocane, mexiletine, tocainide, phenytoin, encainide, felcainide, moricizine, propafenone, riluzole, any combinations thereof, and/or the like.

Further, embodiments provides huperzine conjugates and huperzine analog conjugates of therapeutic agents including a potassium channel blockers, a pro-drug of the potassium channel blockers, a precursor of the potassium channel blockers, or combinations thereof. Examples of potassium channel blockers which may be conjugated to huperzine or an analog thereof include, but are not limited to dofetilide, sotalol, ibutilide, azimilide, bretylium, clofilium, E-4031, nifekalant, tedisamil, sematilide, 4-aminopyridine, and 3,4-diaminopyridine; a pro-drug of the potassium channel blocker; a precursor of the potassium channel blocker; and any combination thereof.

In addition, embodiments provide huperzine conjugates and huperzine analog conjugates of therapeutic agents including an anticonvulsant agent; a pro-drug of the anticonvulsant agent, and a precursor of the anticonvulsant agent. Examples of anticonvulsant agents which may be conjugated to huperzine or an analog thereof include, but are not limited to, (S)-pregabilin, pregabilin, gabapentin, paraldehyde, stiripentol, phenobarbital, methylphenobarbital, barbexaclone, clobazam, clonazepam, clorazepate, diazepam, midazolam, lorazepam, nitrazepam, temazepam, nimetazepam, potassium bromide, felbamate, carbamazepine, oxcarbazepine, eslicarbazepine acetate, valproic acid, sodium valproate, divalproex sodium, vigabatrin, progabide, tiagabine, topiramate, ethotoin, phenytoin, mephenytoin, fosphenytoin, pheneturide, paramethadione, trimethadione, ethadione, beclamide, primidone, brivaracetani, levetiracetam, seletracetam, ethosuximide, phensuximide, mestiximide, acetazolamide, sultiame, phenacemide, methazolamide, zonisamide, lamotrigine, any combination thereof, and/or the like.

Still other embodiments provide huperzine conjugates and huperzine analog conjugates of therapeutic agents including an anxiolytic agent, a pro-drug of the anxiolytic agent, and a precursor to the anxiolytic agent. Examples of anxiolytic agents which may be conjugated to huperzine or an analog thereof include, but are not limited to, positive allosteric modulators of GABA receptor, serotonin-specific reuptake inhibitors (SSRI), barbiturates, benzodiazepines, ethanol, carisoprodol, etomidate, glutethitnide, kavalactones, meprobamate, methaqualone, neuroactive steroids, propofol, theanine, hydroxyzine, valerenic acid, niacin/niacinamide, and any combination thereof.

Yet other embodiments provide huperzine conjugates and huperzine analog conjugates of therapeutic agents including an NADPH oxidase inhibitor, a pro-drug of the NADPH oxidase inhibitor agent, and a precursor to the NADPH oxidase inhibitor agent. Examples of NADPH oxidase inhibitor agents which may be conjugated to huperzine or an analog thereof include, but are not limited to, apocynin, diphenylene iodonium, and any combination thereof.

Yet other embodiments provide huperzine conjugates and huperzine analog conjugates of therapeutic agents including a gamma amino butyric acid (GABA) reuptake inhibitor agent, a pro-drug of the gamma amino butyric acid (GABA) reuptake inhibitor agent, and a precursor to the gamma amino butyric acid (GABA) reuptake inhibitor agent. Examples of gamma amino butyric acid (GABA) reuptake inhibitor agents Which may be conjugated to huperzine or an analog thereof include, but are not limited to, nipecotic acid and any combination thereof.

Further, embodiments provides huperzine conjugates and huperzine analog conjugates of therapeutic agents including an monoamine oxidase B (MAO-B) inhibitor agent, a pro-drug of the monoamine oxidase B (MAO-B) inhibitor agent, a precursor of the monoamine oxidase B (MAO-B) inhibitor agent, or combinations thereof. Examples of monoamine oxidase B (MAO-B) inhibitor agents which may be conjugated to huperzine or an analog thereof include, but are not limited to, lazabemide, pargyline, rasagiline, selegiline, entacapone tolcapone, nitecapone, and quercetin, any combinations thereof, and/or the like.

Further, embodiments provides huperzine conjugates and huperzine analog conjugates of therapeutic agents including a muscarinic receptor antagonist agent, a pro-drug of the muscarinic receptor antagonist agent, a precursor of the muscarinic receptor antagonist agent, or combinations thereof. Examples of muscarinic receptor antagonist agents which may be conjugated to huperzine or an analog thereof include, but are not limited to, atropine, cycycloverine, diphenhydramine, toltcrodine, oxybutynin, opratropium, chlorportnazine, methoctramine, tripitramine, and gallamine, any combinations thereof, and/or the like.

Further, embodiments provides huperzine conjugates and huperzine analog conjugates of therapeutic agents including a dopamine receptor antagonist agent, a pro-drug of the dopamine receptor antagonist agent, a precursor of the dopamine receptor antagonist agent, or combinations thereof. Examples of dopamine receptor antagonist agents which may be conjugated to huperzine or an analog thereof include, but are not limited to, malperone, risperidone, ziprasidone, raclopride, clozapine, haloperidol, quetiapine, domperidone, eticlopride, yohimbine, blonanserin and L-741,626 (3-[4-(4-Chlorophenyl)-4-hydroxypiperidin-1-yl]methyl-1H-indole), any combinations thereof, and/or the like.

Further, embodiments provides huperzine conjugates and huperzine analog conjugates of therapeutic agents including a glutamate receptor (NR2B) antagonist agent, a pro-drug of the glutamate receptor (NR2B) antagonist agent, a precursor of the glutamate receptor (NR2B) antagonist agent, or combinations thereof. Examples of glutamate receptor (NR2B) antagonist agents which may be conjugated to huperzine or an analog thereof include, but are not limited to, ifenprodil, any combinations thereof, and/or the like.

Further, embodiments provides huperzine conjugates and huperzine analog conjugates of therapeutic agents including epigallocatechin gallate (EGCG), a pro-drug of epigallocatechin gallate, a precursor of epigallocatechin gallate, or combinations thereof.

Further, embodiments provides huperzine conjugates and huperzine analog conjugates of therapeutic agents including a aromatase inhibitor agent, a pro-drug of the aromatase inhibitor agent, a precursor of the aromatase inhibitor agent, or combinations thereof. Examples of aromatase inhibitor agents which may be conjugated to huperzine or an analog thereof include, but are not limited to, aminoglutethimide and formestane, any combinations thereof, and/or the like.

Another aspect is a composition comprising a huperzine conjugate or huperzine analog conjugate of the various formula I or formula Ia embodiments, and further comprising a pharmaceutically acceptable excipient in a pharmaceutical composition. In certain embodiments, the composition is administered via a route selected from orally, nasally, rectally, intravenously, intrathecally, intramuscularly, and combinations thereof. In still other embodiments, the composition is administered in a form selected from a sponge, an ointment, a paste, a spray, a patch, a cream, a gel, a foam, a tablet, a capsule, an aqueous solution, an aqueous mixture, an aqueous colloid, an emulsion, a pump, a biodegradable implantable device, a sustained release vehicle and combinations thereof.

From certain embodiments of compounds as conjugates of formula I or conjugates of formula Ia are subembodiments providing the conjugate is not one of the following formulas:

R₁=—CH₃,R₂=—CH₃, and n=1;

R₁=—CF₃,R₂=—CF₃, and n=1;

R₂=—CF₃,R₂=—CF₃, and n=1;

R₂=—CH₃,R₂=—CF₃, and n=; or

R₁=—CH₃,R₂=-phenyl, and n=1.

From certain embodiments of pharmaceutical compositions are subembodiments wherein the conjugate is not one of the following formulas:

R₁=—CH₃,R₂=—CH₃, and n=1;

R₁=—CF₃,R₂=—CH₃, and n=1;

R₁=—CF₃,R₂=—CF₃, and n=1;

R₁=—CH₃,R₂=—CF₃, and n=1; or

R₁=—CF₃,R₂=-phenyl, and n=1.

From certain embodiments of methods using conjugates to treat neurodegenerative diseases by administering a therapeutically effective amount of the conjugate, wherein the neurodegenerative disease is treated, are subembodiments wherein the conjugate is not one of the following formulas:

R₁=—CH₃,R₂=—CH₃, and n=1;

R₁=—CF₃,R₂=—CH₃, and n=1;

R₁=—CF₃,R₂=—CF₃, and n=1;

R₁=—CH₃,R₂=—CF₃, and n=1; or

R₁=—CH₃,R₂=-phenyl, and n=1.

From certain embodiments of methods using conjugates to deliver a therapeutic agent, are subembodiments wherein the conjugate is not one of the following formulas:

R₁=—CH₃,R₂=—CH₃, and n=1;

R₁=—CF₃,R₂=—CF₃, and n=1;

R₁=—CF₃,R₂=—CF₃, and n=1;

R₁=—CH₃,R₂=—CF₃, and n=1; or

R₁=—CH₃,R₂=-phenyl, and n=1.

From certain embodiments of methods using conjugates to increase the concentration of a therapeutic agent in a target organ, are subembodiments wherein the conjugate is not one of the following formulas:

R₁=—CH₃,R₂=—CH₃, and n=1;

R₁=—CF₃,R₂=—CF₃, and n=1;

R₁=—CF₃,R₂=—CF₃, and n=1;

R₁=—CH₃,R₂=—CF₃, and n=1; or

R₁=—CH₃,R₂=-phenyl, and n=1.

From certain embodiments of compounds as conjugates of formula I or formula Ia, are subembodiments providing the conjugate does not include wherein -T is a protein.

From certain embodiments of pharmaceutical compositions are subembodiments of the composition embodiments, wherein the conjugate does not include wherein T is a protein.

From certain embodiments of methods using conjugates to treat neurodegenerative diseases by administering a therapeutically effective amount of the conjugate, are subembodiments of the various methods wherein the conjugate does not include wherein -T is a protein.

From certain embodiments of methods using conjugates to deliver a therapeutic agent, are subembodiments of the various methods wherein the conjugate does not include wherein -T is a protein.

From each of the embodiments of methods using conjugates to increase the concentration of a therapeutic agent in a target organ, are subembodiments of the various methods wherein the conjugate does not include wherein -T is a protein.

The embodiments thereof illustrating the method and materials used may be further understood by reference to the following non-limiting examples. This invention and embodiments illustrating the method and materials used may be further understood by reference to the following non-limiting examples.

### EXAMPLES

### Example 1

### (S)-Pregabilin Conjugated to Huperzine at R₅

The compound wherein huperzine is conjugated to a therapeutic agent to give the compound having structure (II):

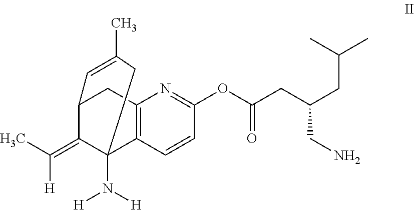 [SMILES: C/C=C1\C2C=C(C)CC1(N)c1ccc(OC(=O)C[C@@H](CN)CC(C)C)nc1C2]

The therapeutic agent is (S)-3-(aminomethyl)-5-methylhexanoic acid, ((S)-Pregabalin) found to activate L-glutamic acid decarboxylase, a CNS active compound having anticonvulsive therapeutic effect. Conjugation is effected by esterifying the (S)-Pregabalin to the R₅ position of huperzine as in Scheme 1:

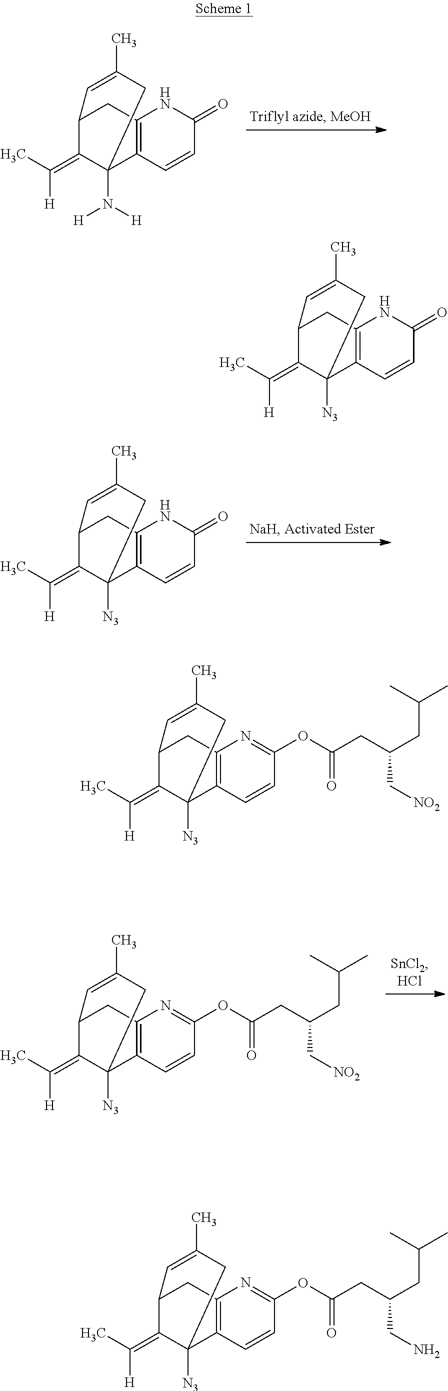

### Example 2

### (S)-Pregabilin Conjugated to Huperzine at R₃

The compound having the formula:

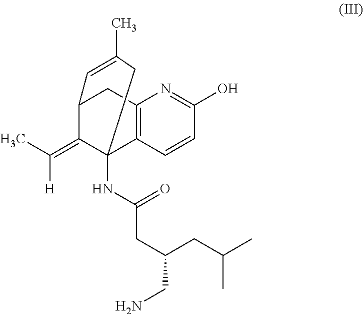 [SMILES: C/C=C1\C2C=C(C)CC1(NC(=O)C[C@@H](CN)CC(C)C)c1ccc(O)nc1C2]

Huperzine is conjugated to the therapeutic agent (S)-Pregabalin to give the compound having structure (III). Conjugation is effected as in Scheme 2 by the formation of an amide bond at R₃ with an activated ester of (S)-Pregabilin.

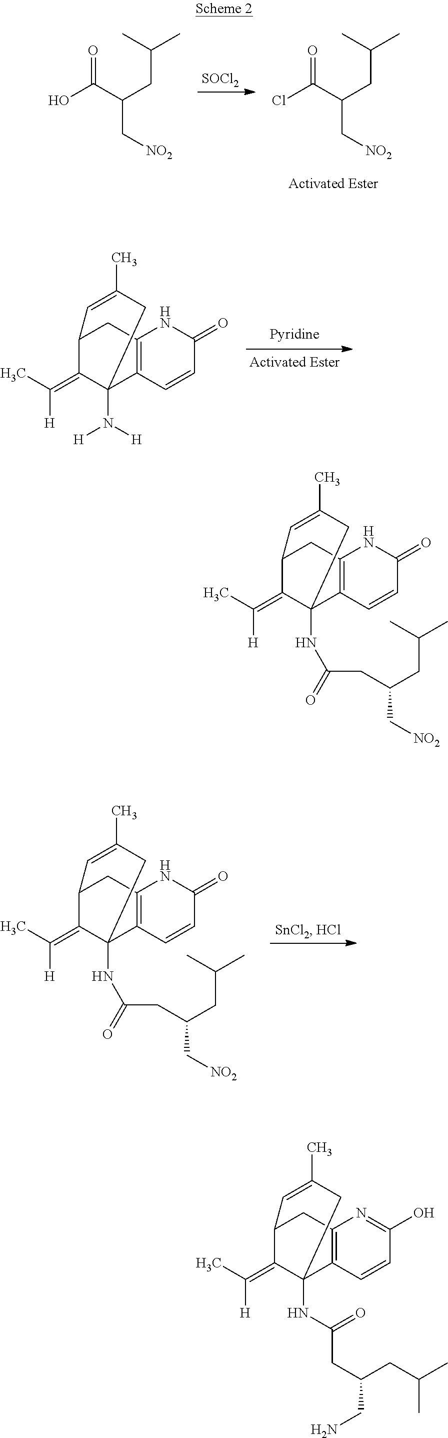

### Example 3

### Riluzole Conjugated to Huperzine at R₄

The compound wherein huperzine is conjugated to a therapeutic agent to give the compound having structure (IV-d):

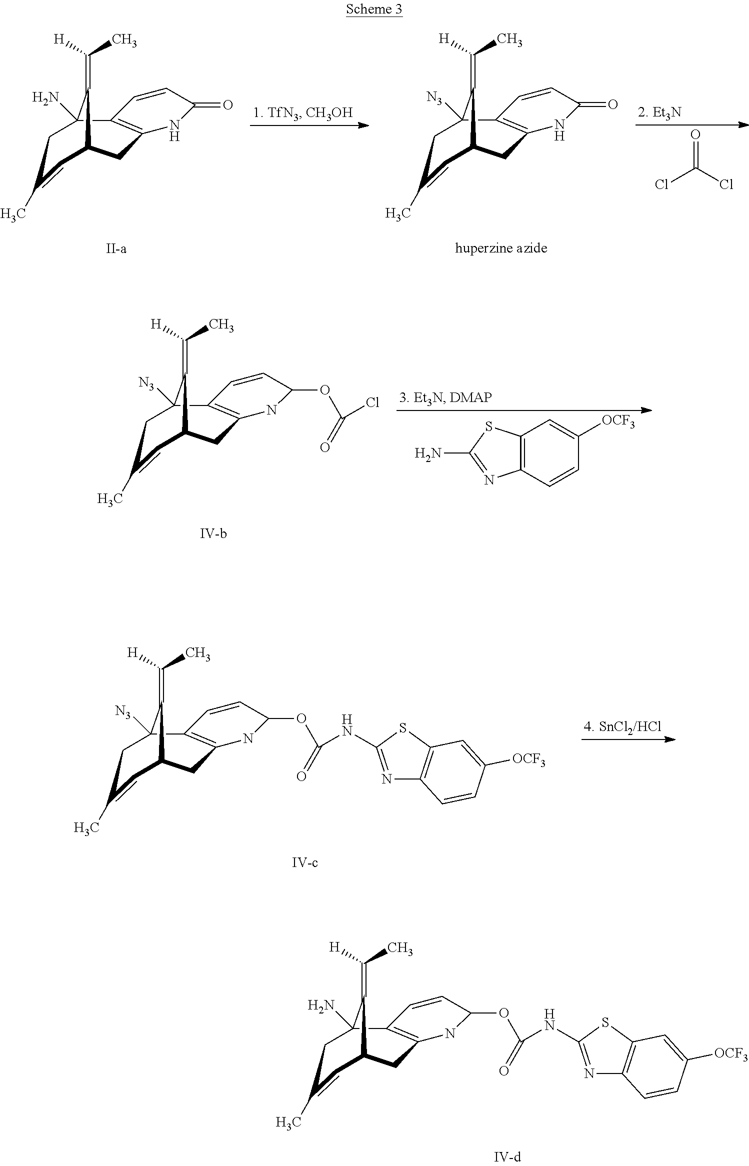

(−)-Huperzine A, (II-a) is reacted with trifle azide in methanol to give huperzine azide. Reaction of huperzine azide with phosgene and triethylamine gives the chloroformate II-b. Reaction of H-b with riluzole and an amine base as in Scheme 3 gives the conjugate azide IV-c. Reduction with stannous chloride and acid gives the riluzole-huperzine conjugate IV-d.

### Example 4

### Apocynin Conjugated to Huperzine at R₄

The compound wherein huperzine is conjugated to a therapeutic agent to give the compound having structure (V-d):

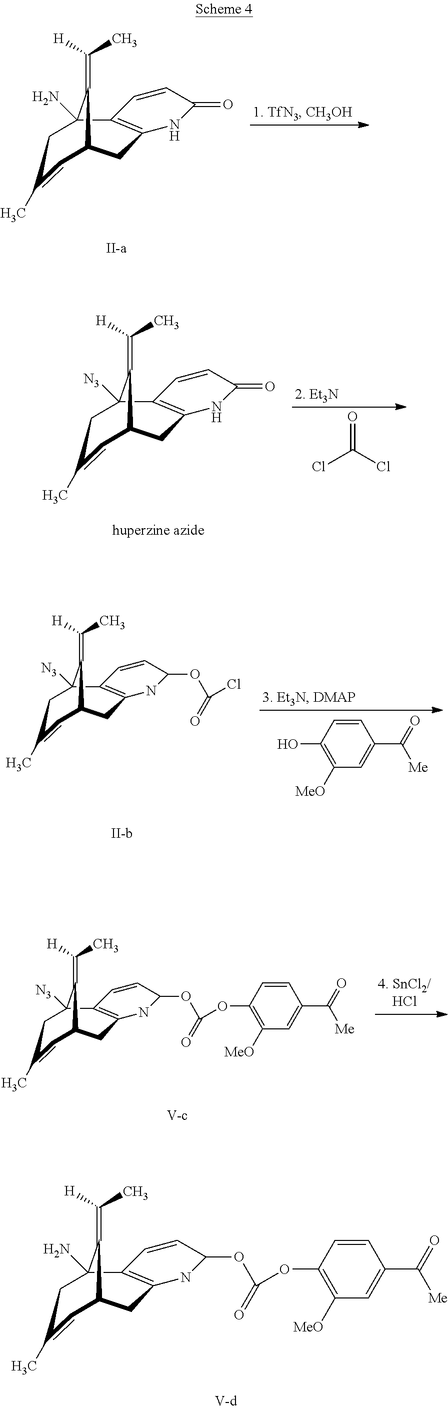

(−)-Huperzine A, (II-a) is reacted with triflic azide in methanol to give huperzine azide. Reaction of huperzine azide with phosgene and triethylamine gives the chloroformate Reaction of II-b with apocynin and an amine base as shown in Scheme 4 gives the conjugate azide V-c. Reduction with stannous chloride and acid gives the apocynin-huperzine conjugate V-d.

### Example 5

### Vanillyl Alcohol Conjugated to Huperzine at R₄

The compound wherein huperzine is conjugated to a therapeutic agent to give the compound having structure (VI-d):

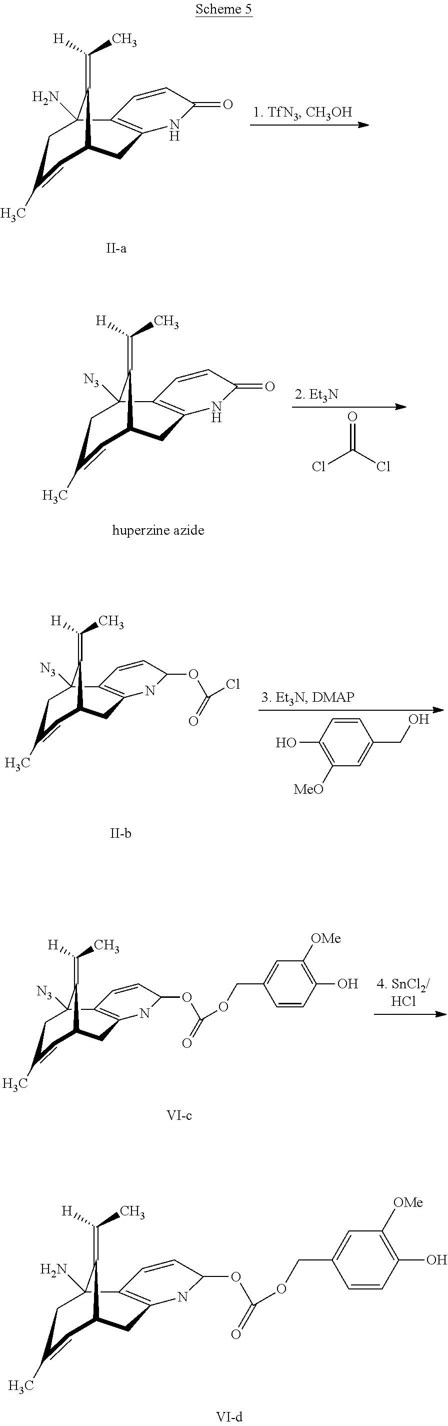

(−)-Huperzine A, (II-a) is reacted with triflic azide in methanol to give huperzine azide. Reaction of huperzine azide with phosgene and triethylamine gives the chloroformate Reaction of II-b with vanillyl alcohol and an amine base as shown in Scheme 5 gives the conjugate azide VI-c. Reduction with stannous chloride and acid gives the vanillyl alcohol-huperzine conjugate VI-d.

### Example 6

### 4-Aminopyridine Conjugated to Huperzine at R₄

The compound wherein huperzine is conjugated to a therapeutic agent to give the compound having structure (VII-d):

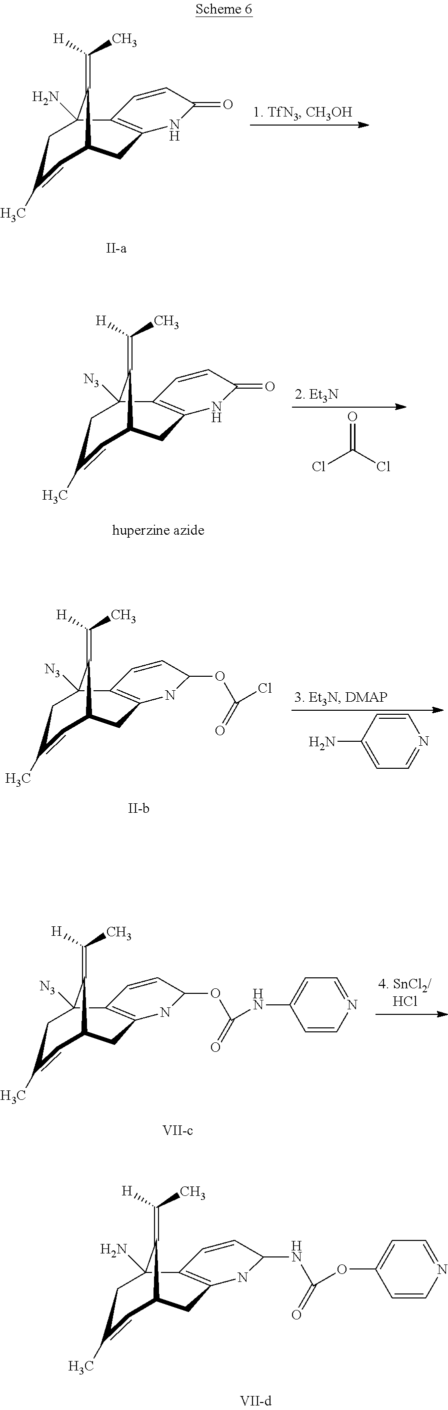

(−)-Huperzine A, (II-a) is reacted with triflic azide in methanol to give huperzine azide. Reaction of huperzine azide with phosgene and triethylamine gives the chloroformate II-b. Reaction of II-b with 4-aminopyridine and an amine base as shown to Scheme 6 Rives the conjugate azide VII-c. Reduction with stannous chloride and acid gives the 4-aminopyridine-huperzine conjugate VII-d.

### Example 7

### 1,3,4-Oxadiazol-2-Amine Conjugated to Huperzine at R₄

The compound wherein huperzine is conjugated to a therapeutic went to give the compound having structure (VIII-d):

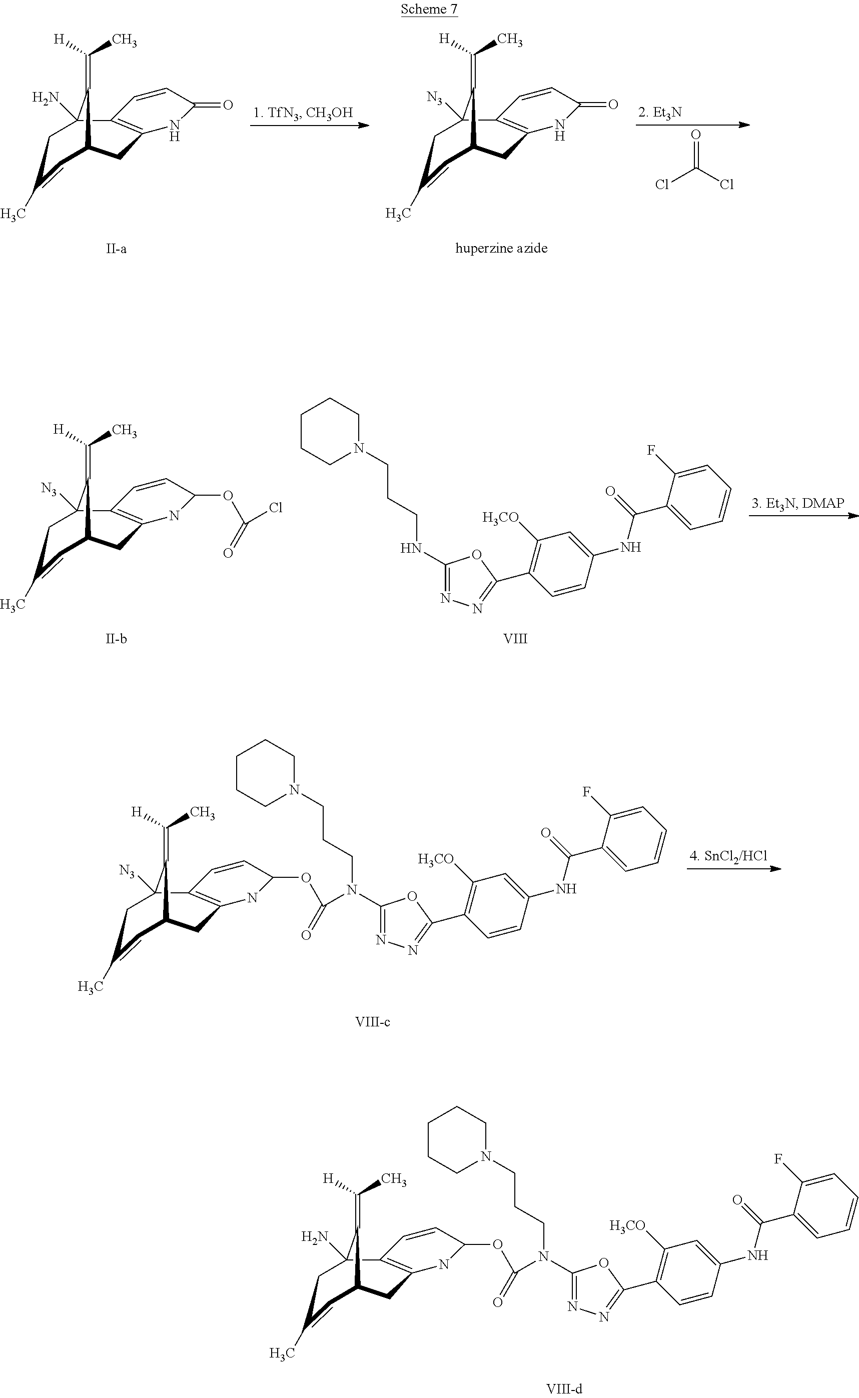

(−)-Huperzine A, (II-a) is reacted with triflic azide in methanol to give huperzine azide. Reaction of huperzine azide with phosgene and triethylamine gives the chloroformate II-b. Reaction of II-b with compound VIII and an amine base as shown. In Scheme 7 gives the conjugate azide VIII-c. Reduction with stannous chloride and acid gives the 1,3,4-oxadoazole-2-amine-huperzine conjugate.

A second aspect of the invention is a method for treating a neurodegenerative disease by administering to a subject in need thereof a therapeutic agent by administration of a conjugate of the therapeutic agent, a pro-drug of the therapeutic agent, or a precursor of the therapeutic agent conjugated to huperzine or a huperzine analog as a composition of the first aspect. In embodiments, the formulation may further include a pharmaceutically acceptable excipient, diluent or carrier.

A third aspect of the invention is a method of delivering a therapeutic agent to a subject in need thereof using a therapeutic agent covalently linked to huperzine or an analog thereof of the fast aspect. The method comprises the administration a compound of the first aspect.

In each of aspects, the compounds or compositions may be administered orally, nasally, rectally, intravenously, intrathecally, intramuscularly, transdermally, opthalmically, and the like. In some embodiments the composition may be administered via a combination of these various routes.

In some embodiments, the compounds or compositions may be administered in the form of a tablet, a capsule, an aqueous solution, an aqueous mixture, an aqueous colloid, a milk, an emulsion, a sponge, an ointment, a paste, a spray, a patch, a cream, a gel, a foam, a pump, a biodegradable implantable device, a sustained release vehicle, and the like. In other embodiments, the composition may be administered as a combination of these various forms.

The compounds or compositions of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds and compositions of the present invention can be formulated for parenteral administration by injection, e.g., by bolus injection or continuous infusion. The compounds or compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds and compositions can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas in the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium Phosphate.

In some embodiments, the diluent component comprises one or more of mannitol, lactose, sucrose, maltodextrin, sorbitol, xylitol, powdered cellulose, microcrystalline cellulose, carboxymethylcellulose, starch, carboxyethylcellulose, pregelatinized starch, sodium starch glycolate, methylcellulose, ethylcellulose, hydroxyethyleellulose, methylhydroxyethylcellulose, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

In some aspects, a method of delivering a therapeutic agent may include covalently linking the therapeutic agent to huperzine or an analog thereof. Various embodiments of a compound comprising a therapeutic agent covalently linked to huperzine or an analog thereof are described herein.

The present disclosure should not be considered limited to the particular embodiments described above, but rather should be understood to cover all aspects of the disclosure as fairly set out in the attached claims. Various modifications as well as numerous structures to which the present disclosure may be applicable, will be readily apparent to those skilled in the art to which the present disclosure is directed upon review of the present specification. The claims are intended to cover such modifications and devices.

What is claimed is:

1. A compound having a general formula: 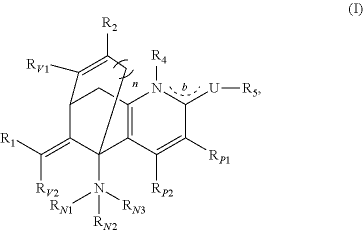
tautomer thereof, or pharmaceutically acceptable salt thereof, wherein R₁ is selected from —H, —(C₁-C₂₄)alkyl, —CF₃, —CF₂CF₃, —CF₂CF₂CF₃, —SO₂CH₃, —SO₂Ph, —SO₂Ar, —SO₃H, and —SO₃Ar, and —CH₂-L-T;
R₂ is selected from —H, —(C₁-C₂₄)alkyl, -aryl, -cycloalkyl, —(C₂-C₂₄)alkenyl, -heterocycle, -heteroaryl, and —CH₂-L-T;
R_{P1}, R_{P2}, R_{V1}, R_{V2} are independently selected from hydrogen and fluorine;
U is O, S, NH, or N((C₁-C₂₄)alkyl);
b is a keto-enol tautomer unsaturation;
R_{N1} is selected from H, —(C₁-C₂₄)alkyl, —CF₃, —CF₂CF₃, —CCl₃, —CBr₃, —CHO, and -L-T;
R_{N2} is selected from H, —(C₁-C₂₄)alkyl, —CF₃, —CF₂CF₃, —CCl₃, —CBr₃, and —CHO;
R_{N3} is selected from absent and -alkyl;
n is an integer selected from 1, 2, 3, and 4;
R₄ is absent, or selected from —H, and -L-T;
R₅ is absent, or selected from —H, and -L-T;
at least one of R₁, R₂, R_{N1}, R₄ and R₅ is -L-T;
each -L- is independently a linker; and
wherein each -T is independently selected from a therapeutic agent, a therapeutic anent pro-drug, or a therapeutic agent precursor.

2. The compound of claim 1, wherein each therapeutic agent, a therapeutic agent pro-drug, or a therapeutic agent precursor is independently —V—W—X—Y—Z, wherein V is bond, —O—, or —NH—;
W is —(C₀-C₆)alkyl-, —(C₂-C₆)alkenyl-; or —(C₂-C₆)alkynyl-;
X is a bond, —O—, —NH—, —CO—, —(C═O)NH—, —NH—(C═O)—, —SO₂—, —(C═NH)—NH—, —(C═O)—O—, or —O(C═O)—;
Y is a —(C₀-C₆)alkyl-, —(C₂-C₆)alkenyl-; or —(C₂-C₆)alkynyl-;
Z is a -quaternary amine, -cycloalkyl, -aryl, -heterocycle, or heteroaryl; and
wherein each nitrogen, alkyl, alkenyl, alkynyl, cycloalkyl, aryl, heterocycle, or heteroaryl is optionally substituted.

3. The compound of claim 1, wherein -L- comprises at least one of a bond, —O—, —S—, —NH—,
—N(alkyl)-, —C(O)—, —O(C═O)—, —C(═O)O—, —C(═S)O—, —C(═S)—, and —P(O)₂—.

4. The compound of claim 1, wherein only one of R₁, R₂, R_{N1}, R₄ and R₅ is -L-T; R₁ is selected from CH₃, CF₃, CF₂CF₃, CF₂CF₂CF₃, SO₂CH₃, SO₂Ph, SO₂Ar, and SO₃H; and R₂ is selected from —(C₁-C₂₄)alkyl, an aryl, a cycloalkyl, an alkenyl, heterocycle, and a heteroaryl.

5. The compound of claim 1, wherein one of R₃, R₄ and R₅ is -L-T;
R₁ is selected from H, CH₃, CF₃, CF₃CF₃, CF₂CF₂CF₃, SO₂CH₃, SO₂Ph, SO₂Ar, and SO₃H; and
R₂ is selected from H, alkyl, aryl, cycloalkyl, alkenyl, heterocycle, and heteroaryl.

6. The compound of claim 1, wherein -L- comprises at least one functional group selected from phosphoramide, phosphoester, carbonate, amide, carboxyiphosphoryl anhydride, thioester, ether, thioether, amine, and carboxylic ester.

7. The compound of claim 1, wherein n=1, and R₁═CH₃ and R₂═CH₃.

8. The compound of claim 1, wherein -T is selected from —(C═O)—CH₂—CH(i-Bu)-CH₂—NH₂ and (S)—(C═O)—CH₂—CH(i-Bu)-CH₂—NH₂.

9. The compound of claim 1, wherein -T is selected from a glutamate receptor antagonist, an N-methyl d-aspartate (NMDA) receptor antagonist, mitochondrial protectant, an anti-inflammatory agent, alpha-7 agonist, potassium channel blocker, sodium channel blacker, anticonvulsant, anxiolytic agent, nicotinamide adenine dinucleotide phosphate (NADPH) oxidase inhibitor; a pro-drug to one of a glutamate receptor antagonist, an N-methyl
d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor; a precursor to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, auxiolytic agent, NADPH oxidase inhibitor, a gamma amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof.

10. A method for treating a disease in a subject comprising administering to the subject a therapeutically effective amount of a compound of the formula: 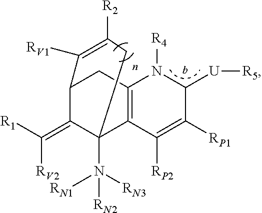
tautomer thereof, or pharmaceutically acceptable salt thereof, wherein R₁ is selected from —H, —(C₁-C₂₄)alkyl, —CF₃, —CF₂CF₃, —CF₂CF₂CF₃, —SO₂CH₃, —SO₂Ph, —SO₂Ar, —SO₃H, and —SO₃Ar, and —CH₂-L-T;
R₂ is selected from —H, —(C₁-C₂₄)alkyl, -aryl, -cycloalkyl, —(C₂-C₂₄)alkenyl, -heterocycle, -heteroaryl, and —CH₂-L-T;
R_{P1}, R_{P2}, R_{V1}, R_{V2} are independently selected from hydrogen and fluorine;
U is O, S, NH, or N((C₁-C₂₄)alkyl);
b is a keto-enol tautomer unsaturation;
R_{N1} is selected from H, —(C₁-C₂₄)alkyl, —CF₃, —CF₂CF₃, —CCl₃, —CBr₃, —CHO, and -L-T;
R_{N2} is selected from H, —(C₁-C₂₄)alkyl, —CF₃, —CF₂CF₃, —CCl₃, —CBr₃, and —CHO;
R_{N3} is selected from absent and -alkyl;
n is an integer selected from 1, 2, 3, and 4;
R₄ is absent, or selected from —H, and -L-T;
R₅ is absent, or selected from —H, and -L-T;
at least one of R₁, R₂, R_{N1}, R₄ and R₅ is -L-T;
each -L- is independently a linker; and
wherein each -T is independently selected from a therapeutic agent, a therapeutic anent pro-drug, or a therapeutic agent precursor.

11. The method of claim 10, wherein each therapeutic agent, the therapeutic agent pro-drug, or a therapeutic agent precursor is independently V—W—X—Y—Z, wherein V is bond, —O—, or —NH—;
W is —(C₀-C₆)alkyl-, —(C₂-C₆)alkenyl-; or —(C₂-C₆)alkynyl-;
X is a bond, —O—, —NH—, —CO—, —(C═O)NH—, —NH—(C═O)—, —SO₂—, —(C═NH)—NH—, —(C═O)—O—, or —O(C═O)—;
Y is a —(C₀-C₆)alkyl-, —(C₂-C₆)alkenyl-; or —(C₂-C₆)alkynyl-;
Z is a -quaternary amine, -cycloalkyl, -aryl, -heterocycle, or heteroaryl; and
wherein each nitrogen, alkyl, alkenyl, alkynyl, cycloalkyl, aryl, heterocycle, or heteroaryl is optionally substituted.

12. The method of claim 10, wherein comprises at least one of a bond, —O—, —S—, —NH—, —N(alkyl)-, —C(O)—, —O(C═O)—, —C(═O)—, —C(═S)O—, —C(═S)—, and —P(O)₂—.

13. The method of claim 10, wherein only one of R₁, R₂, R_{N1}, R₄ and R₅ is -L-Tl R₁ is selected from CH₃, CF₃, CF₂CF₃, CF₂CF₂CF₃, SO₂CH₃, SO₂Ph, SO₂Ar, and SO₃H; and R₂ is selected from —(C₁-C₂₄)alkyl, an aryl, a cycloalkyl, an alkenyl, a heterocycle, and a heteroaryl.

14. The method of claim 10, wherein one of R₃, R₄ and R₅ is -L-T;
R₁ is selected from H, CH₃, CF₃, CF₂CF₃, CF₂CF₂CF₃, SO₂CH₃, SO₂Pb, SO₂Ar, and SO₃H; and
R₂ is selected from H, alkyl, aryl, cycloalkyl, alkenyl, heterocycle, and heteroaryl.

15. The method of claim 10, wherein -L- comprises at least one functional group selected from phosphoramide, phosphoester, carbonate, amide, carboxylphosphoryl anhydride, thioester, ether, thioether, amine, and, carboxylic ester.

16. The method of claim 10, wherein n=1, and R₁═CH₃ and R₂═CH₃.

17. The method of claim 10, wherein is selected from —(C═O)—CH₂—CH(i-Bu)-CH₂—NH₂ and (S)—(C═O)—CH₂—CH(i-Bu)-CH₂—NH₂.

18. The method of claim 10, wherein T is selected from a glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl
d-aspartate receptor antagonist, mitochondrial protectant, an anti-inflammatory agent, alpha-7 agonist, potassium channel Mocker, sodium channel blocker, anticonvulsant, anxiolytic agent, NADPH oxidase inhibitor; a precursor to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor, a gamma amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof.

19. The method of claim 10, wherein the disease is a neurodegenerative disease selected from Alzheimer's disease, epilepsy, neuropathic pain, multiple sclerosis, Parkinson's disease, ataxia, Huntington's disease, amyotrophic lateral sclerosis, AIDS-related dementia, neurotoxic poisoning, infantile spasms, and combinations thereof′.

20. The method of claim 10 further comprising co-administration of an antioxidant agent, an anti-inflammatory agent, or combinations thereof.

21. A method of delivering a therapeutic agent comprising administering to a subject a compound having a general formula: 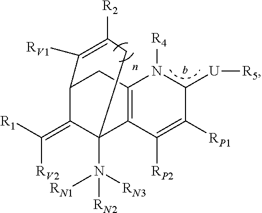
tautomer thereof, or pharmaceutically acceptable salt thereof, wherein R₁ is selected from —H, —(C₁-C₂₄)alkyl, —CF₃, —CF₂CF₃, —CF₂CF₂CF₃, —SO₂CH₃, —SO₂Ph, —SO₂Ar, —SO₃H, and —SO₃Ar, and —CH₂-L-T;
R₂ is selected from —H, —(C₁-C₂₄)alkyl, -aryl, -cycloalkyl, —(C₂-C₂₄)alkenyl, -heterocycle, -heteroaryl, and —CH₂-L-T;
R_{P1}, R_{P2}, R_{V1}, R_{V2} are independently selected from hydrogen and fluorine;
U is O, S, NH, or N((C₁-C₂₄)alkyl);
b is a keto-enol tautomer unsaturation;
R_{N1} is selected from H, —(C₁-C₂₄)alkyl, —CF₃, —CF₂CF₃, —CCl₃, —CBr₃, —CHO, and -L-T;
R_{N2} is selected from H, —(C₁-C₂₄)alkyl, —CF₃, —CF₂CF₃, —CCl₃, —CBr₃, and —CHO;
R_{N3} is selected from absent and -alkyl;
n is an integer selected from 1, 2, 3, and 4;
R₄ is absent, or selected from —H, and -L-T;
R₅ is absent, or selected from —H, and -L-T;
at least one of R₁, R₂, R_{N1}, R₄ and R₅ is -L-T;
each -L- is independently a linker; and
wherein each -T is independently selected from a therapeutic agent, a therapeutic anent pro-drug, or a therapeutic agent precursor.

22. The method of claim 21, wherein each T is independently V—W—X—Y—Z, wherein V is bond, —O—, or —NH—;
W is —(C₀-C₆)alkyl-, —(C₂-C₆)alkenyl-; or —(C₂-C₆)alkynyl-;
X is a bond, —O—, —NH—, —CO—, —(C═O)NH—, —NH—(C═O)—, —SO₂—, —(C═NH)—NH—, —(C═O)—O—, or —O(C═O)—;
Y is a —(C₀-C₆)alkyl-, —(C₂-C₆)alkenyl-; or —(C₂-C₆)alkynyl-;
Z is a -quaternary amine, -cycloalkyl, -aryl, -heterocycle, or heteroaryl; and
wherein each nitrogen, alkyl, alkenyl, alkynyl, cycloalkyl, aryl, heterocycle, or heteroaryl is optionally substituted.

23. The method of claim 21, wherein comprises at least one of a bond, —O—, —S—, —N—, —N(alkyl)-, —C(O)—, —O(C═O)—, —C(═S)O—, —C(═S)—, and —P(O)₂—.

24. The method of claim 21, wherein only one of R₁, R₂, R_{N1}, R₄ and R₅ is -L-T; R₁ is selected from CH₃, CF₃, CF₂CF₃, CF₂CF₂CF₃, SO₂CH₃, SO₂Ph, SO₂Ar, and SO₃H; and R₂ is selected from —(C₁-C₂₄)alkyl, an aryl, a cycloalkyl, an alkenyl, heterocycle, and a heteroaryl.

25. The method of claim 21, wherein one of R₃, R₄ and R₅ is -L-T;
R₁ is selected from H, CH₃, CF₃, CF₂CF₃, CF₂CF₂CF₃, SO₂CH₃, SO₂Ph, SO₂Ar, and SO₃H; and
R₂ is selected from H, alkyl, aryl, cycloalkyl, alkenyl, heterocycle, and heteroaryl.

26. The method of claim 21, wherein -L- comprises at least one functional group selected from phosphoramide, phosphoester, carbonate, amide, carboxylphosphoryl anhydride, thioester, ether, thioether, amine, and carboxylic ester.

27. The method of claim 21, wherein n=1, and R₁═CH₃ and R₂═CH₃.

28. The method of claim 21, wherein -T is selected from —(C═O)—CH₂—CH(i-Bu)-CH₂—NH₂and (S)—(C═O)—CH₂—CH(i-Bu)-CH₂—NH₂.

29. The method of claim 21, wherein T is selected from a glutamate receptor antagonist, an N-methyl d-aspartate (NMDA) receptor antagonist, mitochondrial protectant, an anti-inflammatory agent, alpha-7 agonist, potassium channel blocker, sodium channel blocker, anticonvulsant, anxiolytic agent, nicotinamide adenine dinucleotide phosphate (NADPH) oxidase inhibitor; a pro-drug to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor; a precursor to one of a glutamate receptor antagonist, an N-methyl d-aspartate receptor antagonist, mitochondrial protectant, an anti-inflammatory agent, alpha-7 agonist, potassium channel blocker, sodium channel blocker, anticonvulsant, anxiolytic agent, NADPH oxidase inhibitor, a ganuna amino butyric acid reuptake inhibitor, a monoamine oxidase B inhibitor, a muscarinic receptor antagonist, a dopamine receptor antagonist, a glutamate receptor (NR2B) antagonist, epigallocatechin gallate, an aromatase inhibitor; and any combination thereof.
